(19) United States
(12) Patent Application Publication
(10) Pub. No.: US 2025/0322955 A1
(43) Pub. Date: Oct. 16, 2025

(54) ANALYTIC PLATFORM USING NPM1-ASSOCIATED GENES INTERACTION NETWORK FOR IDENTIFYING GENETIC TRAITS

(71) Applicant: B.Y. QUANTITATIVE MEDICINE LIMITED, Hong Kong (CN)

(72) Inventor: Yat-ming YUNG, Hong Kong (CN)

(21) Appl. No.: 19/096,301

(22) Filed: Mar. 31, 2025

Related U.S. Application Data
(63) Continuation-in-part of application No. 18/692,344, filed on Mar. 15, 2024, now abandoned.
(60) Provisional application No. 63/308,067, filed on Feb. 9, 2022.

(51) Int. Cl. G16H 50/20; G06N 20/20; G16B 20/20; G16B 30/10; G16B 40/20; G16H 20/17; G16H 50/70

(52) U.S. Cl. CPC G16H 50/20; G06N 20/20; G16B 20/20; G16B 30/10; G16B 40/20; G16H 20/17; G16H 50/70

(57) ABSTRACT

The invention provides a method and system for analyzing dysregulated biological pathways associated with states of interest to identify risks and molecular targets for personalized treatment. The method employs a two-layer machine learning model (MLM) to assign dysregulated pathway (DP) scores to biological pathways derived from both whole-genome co-expression network analysis and differential gene expression analysis. In the first layer, classifiers are used to predict states based on the identified pathways. In the second layer, a stacking classifier integrates these predictions to compute the final state. Each pathway is weighted according to its contribution to the state of interest, and pathway scores are normalized to reflect their relative significance. The method incorporates the Shapley Additive Explanations (SHAP) technique to enhance model interpretability. This enables the identification of key genes and molecular targets. This method and system are patient-independent, offering a framework for precision medicine across a wide range of conditions.

### CROSS-REFERENCE TO RELATED APPLICATIONS

This application is a continuation-in-part of U.S. Ser. No. 18/692,344, filed Mar. 15, 2024, National Stage of International Application No. PCT/IB2023/051145, filed Feb. 9, 2023, which claims benefit of U.S. Ser. No. 63/308,067, filed Feb. 9, 2022. The contents of these preceding applications are hereby incorporated in their entireties by reference into this application. Throughout this application, various publications are cited. The disclosures of these publications in their entireties are hereby incorporated by reference into this application to more fully describe the state of the art to which this invention pertains.

### FIELD OF THE INVENTION

The present invention relates to a platform that utilizes artificial intelligence to analyze dysregulated biological pathways, with the aim of identifying state of interest risks and molecular targets for developing personalized drug treatment regimens for patients.

### BACKGROUND OF THE INVENTION

Current diagnostic and therapeutic interventions often analyze disease biomarkers in isolation. However, diseases such as cancer exhibit a remarkable ability to adapt through various survival mechanisms, resulting in drug resistance and disease relapse. This challenge arises from researchers' tendency to overlook the complex network of molecular interactions within human cells. By identifying dysregulated interactions specific to disease cells, researchers can precisely target these disease-associated interactions, thereby addressing existing gaps in medical efficacy. Additionally, artificial intelligence can facilitate the screening and analysis of vast datasets, enabling the prediction of the most significant disease-associated interactions for individual patients.

### SUMMARY OF THE INVENTION

The invention provides a method for identifying genetic traits associated with states of interest by comparing genome-wide gene expression and co-expression changes between two distinct cellular states. The resulting gene sets undergo functional enrichment analysis, yielding dysregulated biological pathways that serve as input for the machine learning model (MLM). The MLM employs a two-layer ensemble approach, with the first layer utilizing various classifiers alongside the dysregulated biological pathways to predict states of interest. Each pathway is assigned a probability of state-of-interest severity, which is then used in the second layer-a stacking classifier that integrates these probabilities to ascertain the final state of interest. Pathways are scored and normalized, where higher scores indicate a greater contribution to the state of interest. Subsequently, all pathways are analyzed to identify critical molecular targets for targeted treatment using the Shapley Additive Explanations (SHAP) method. The SHAP method enhances model interpretability by quantifying the contribution of each gene expression feature to state of interest classification. Genes identified as contributing positively may serve as potential targets for therapeutic interventions.

### BRIEF DESCRIPTION OF THE FIGURES

FIG. 1 shows the data selection and feature selection of the current invention.

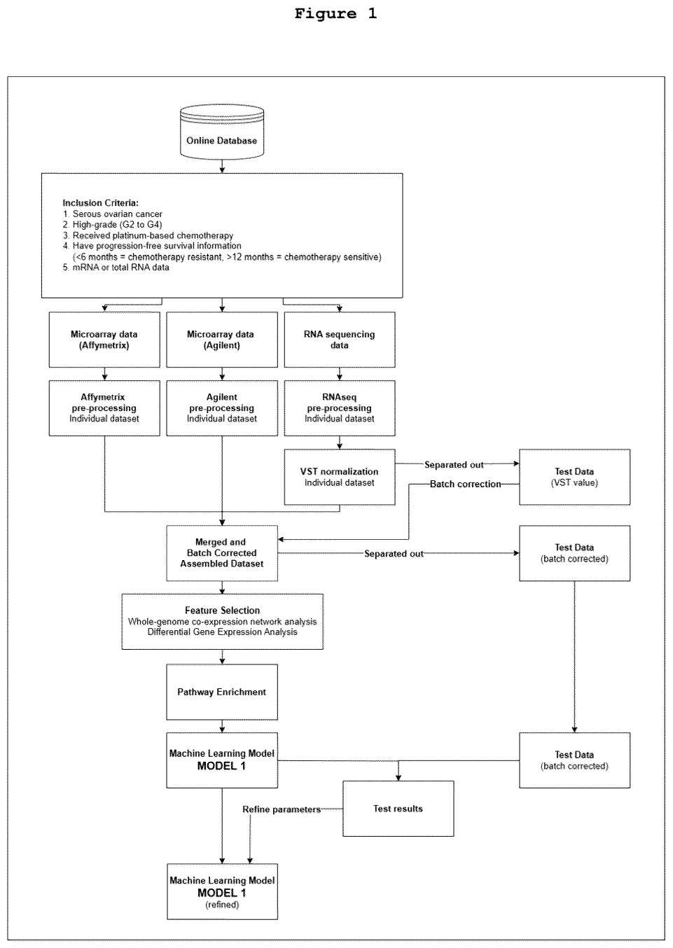

FIG. 2 shows the machine learning model of the current invention.

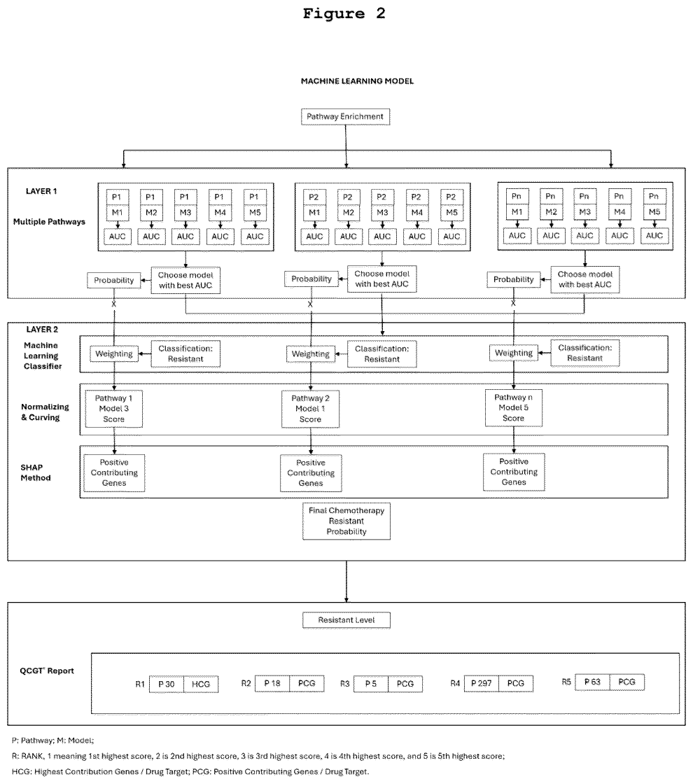

### DETAILED DESCRIPTION OF THE INVENTION

The present invention comprises a big data analytics platform specifically designed to analyze whole-genome co-expression changes and aberrant gene expression patterns associated with various diseases, enabling the identification of dysregulated biological pathways. Furthermore, the platform leverages artificial intelligence to examine these dysregulated pathways, allowing for the identification of state of interest risks and significant molecular targets for tailored therapeutic interventions. This advancement aims to enhance the efficacy of personalized medicine by delivering targeted treatment strategies.

This invention provides a method for identifying a genetic trait of cells in a state of interest.

In one embodiment, the method comprises the steps of: a. receiving a first gene expression dataset from cells in a state of interest; b. receiving a second gene expression dataset from cells in a reference state; c. detecting dysregulated gene sets related to the state of interest using whole-genome co-expression network analysis and differential gene expression analysis; d. generating state-specific pathways using functional enrichment analysis on said dysregulated gene sets; e. generating a dysregulated pathway score for each state-specific pathway using a machine learning model comprising a two-layer ensemble approach, wherein: i. a first layer predicts states of interest based on the state-specific pathways using classifiers selected based on optimal performance metrics: ii. each state-specific pathway is associated with a state of interest severity probability in the first layer; iii. a second layer integrates probabilities from the first layer and computes a final state of interest classification using a stacking classifier: iv. the severity probability of each state-specific pathway is used to assign a weight to that state-specific pathway in the final classification; and v. the weight of each state-specific pathway is multiplied by that state-specific pathway's probability, generating a dysregulated pathway score for each state-specific pathway: f. scaling and normalizing said dysregulated pathway scores, wherein higher scores indicate a greater likelihood of contribution to the state of interest; and g. generating values indicating the impact of each gene on the state-specific pathway's contribution to the final state of interest classification at the model-wide and sample-specific levels.

In one embodiment, the method comprises the steps of:
a. Receiving a first gene expression dataset from cells in a state of interest;
b. Receiving a second gene expression dataset from cells in a reference state:
c. Pre-processing the expression datasets prior to analysis, including but not limited to quality control, background correction, normalization, sequence alignment, gene count quantification, and gene annotation assignment according to the platform used:
d. Utilizing packages for pre-processing, including but not limited to ‘GEOquery,’ ‘limma,’ ‘AnnotationDbi,’ ‘org.Hs.eg.db,’ ‘oligo,’ ‘DESeq2,’ ‘fastp,’ ‘STAR,’ and ‘RSEM’;
e. Applying pre-processing packages at their default settings, except for the following:
   i. In ‘fastp,’ the minimum length of transcripts is set to 40, with auto adapter detection for paired-end sequencing:
f. Scaling each dataset using the ‘StandardScaler’ package, merging all datasets via feature (i.e., gene or protein) alignment, and performing batch correction using the ‘reCombat’ package;
g. Confirming the successful removal of batch effects by employing techniques such as PCA and t-SNE, which segregate datasets by biological clusters rather than by batch differences, and by further validating that all batch effects have been removed using the K-BET score test;
h. Identifying dysregulated gene sets related to the state of interest by:
   i. Conducting a whole-genome co-expression network analysis, evaluating the maximum difference between the data distribution curves of the state of interest and the reference state to identify the threshold value for determining significant gene pair co-expressions; and
   ii. Performing a differential gene expression analysis using a linear modeling approach, including but not limited to the ‘linuna’ package, where genes in the state of interest are considered dysregulated if their expression levels significantly differ from those in the reference state, applying an adjusted p-value threshold of less than 0.05 and filtering genes based on log fold change;
i. Analyzing dysregulated genes to determine whether they are significantly over-represented (enriched) in specific gene sets known as “state-specific pathways”;
j. Utilizing all identified state-specific pathways in a machine learning model employing a two-layer ensemble approach, also known as a stacking classifier, wherein:
   i. The base learner consists of multiple individual models, including but not limited to Support Vector Machine, Light Gradient-Boosting Machine, Extreme Gradient Boosting, Gradient Boosting, Random Forest, Logistic Regression, Gaussian Naive Bayes, Multi-Layer Perceptron, Hist Gradient Boosting, K-Nearest Neighbors, Catboost, Stochastic Gradient Descent, Linear Discriminant Analysis, Adaptive Boosting, and Decision Tree:
   ii. Each state-specific pathway is paired with five different models, using the expression values from each pathway for prediction;
   iii. Hyperparameters are tuned to optimize performance for each model, with the best-performing model (i.e., with the highest AUC) selected to train the next layer (i.e., the meta-model);
   iv. The meta-model takes the probabilities from each best-performing model in the base learner as input and combines them to make a final prediction;
   v. Hyperparameters for tuning in each model comprise: for the Support Vector Machine, the kernel, gamma, and C; for the Light Gradient-Boosting Machine, the learning rate and max depth; for Extreme Gradient Boosting, the learning rate and max depth; for Gradient Boosting, the max depth, max features, min samples leaf, min samples split, n estimators, subsample, and max leaf nodes; for Random Forest, the max depth, max features, min samples leaf, min samples split, and n estimators; for Logistic Regression, the penalty, tolerance, C, solver, and max iterations; for Gaussian Naive Bayes, the variance smoothing; for Multi-layer Perceptron, the alpha and learning rate initialization; for Hist Gradient Boosting, the max depth, max features, min samples leaf, learning rate, max iterations, and max leaf nodes; for K-Nearest Neighbors, the n neighbors and weights; for Catboost, the iterations, use best model, learning rate, depth, random strength, max leaves, and min data in leaf; for Stochastic Gradient Descent, the penalty and max iterations; for Linear Discriminant Analysis, the shrinkage and priors; for Adaptive Boosting, the learning rate, n estimators, and algorithm; and for Decision Tree, the max depth, min samples split, max features, max leaf nodes, and class weight:
   vi. The severity probability of each state-specific pathway is used to assign a weight to that pathway in the final classification;
   vii. The weight of each state-specific pathway is multiplied by its probability, generating a Dysregulated Pathway Score (DPS) for each pathway;
   viii. The meta-model used is a Support Vector Machine, which employs a linear method to calculate the final prediction:
k. Performing model validation using k-fold cross-validation, wherein the training dataset is divided into five subsets (folds), and each fold serves as a test set once while the remaining four folds are used for training, the model is trained on the four folds and evaluated on the fold left out, this process is repeated four times, with each fold used exactly once as the test set;
l. Averaging the performance metrics (i.e., AUC) from all five iterations to provide a single, reliable estimate of the model's performance, resulting in an average AUC of 0.7, indicating that the model can distinguish between the two classes 70% of the time:
m. Normalizing the DPS to a range of 1 to 100 using the min-max normalization method; and
n. Curving the DPS using a square root transformation to reduce skewness and enhance interpretability.

In order to identify target genes for each sample, various methods and criterions can be implemented to make personalized suggestions on which gene should be targeted for a better chance of improved treatment outcome. Local explanations for individual predictions by employing model-agnostic techniques would reveal how each gene may be contributing to the treatment outcome for each patient, which can be the basis of selecting personalized gene target for each sample. Any quantifiable criterion can be used as long as they are supported with a logical hypothesis that would suggest an improved treatment outcome by targeting the gene selected from the criterion. For example, one could recommend targeting genes with positive contribution to predicting the undesirable treatment outcome. Alternatively, one could recommend targeting upregulated genes with positive contribution to predicting the undesirable treatment outcome. This can be implemented at any levels of the Ensemble Classifier, including the first layer pathway models, the second layer model or the entire classifier. The following provide detailed description of possible implementations:

SHAP assigns each feature an importance value for a particular prediction based on cooperative game theory. It calculates Shapley values by considering all possible feature combinations and their contributions to the model's output.

For every model in the Ensemble Classifier, Shapley values can be calculated or estimated using any compatible algorithms with the model, including but not limited to: Additive Explainer, Deep Explainer, Exact Explainer, GPU Tree Explainer, Gradient Explainer, Kernel Explainer, Linear Explainer, Partition Explainer, Permutation Explainer, Sampling Explainer, Tree Explainer.

To calculate Shapley values for n samples with m features, one should prepare a trained model, sufficient or all samples from the training matrix, and the matrix of interest of dimension n×m. Then provide them to a compatible explainer with the appropriate arguments.

If the model makes prediction in log-odds space, use an identity link function, otherwise if the model make prediction in probability space, use a logit link function to convert the probability into log-odds scale.

In the case of binary classification, this should obtain a n×m matrix of Shapley values, which are the calculated or approximated contributions of each feature for each sample towards predicting one of the two classes.

Based on the calculated values and other available information such as the model input values, one may apply their selection method to select any number of features that meet the criteria. For example, one may select all features with positive Shapley value contributing to the undesirable outcome prediction. Finally, the interpretation for each model would be based on the model input feature. For instance, each layer 1 pathway model would suggest potential gene targets, the entire model would also suggest potential gene targets, while the layer 2 meta model would suggest which layer 1 model output are contributing most to the final risk score.

Local Interpretable Model-Agnostic Explanations (LIME) provides local interpretability by approximating the model's behavior around a specific sample with a simpler, interpretable model. It does this by perturbing the sample's features and training a local surrogate model to mimic the original model's behavior within a small region around the sample.

For every model in the Ensemble Classifier, local explanations can be obtained using any LIME Explainer. LIME generates a local surrogate model (like linear regression or decision tree) to explain the model's prediction for a specific instance. The coefficients from the surrogate model indicate the importance of each feature for the given sample.

To calculate local explanation for n samples with m features, one should prepare a trained model, sufficient or all samples from the training matrix with their corresponding labels, and the matrix of interest of dimension n×m. Then provide them to a compatible explainer with the appropriate arguments.

In the case of binary classification, this should obtain a n×m matrix of local explanations, which are the calculated or approximated contributions of each feature for each sample towards predicting one of the two classes.

Based on the calculated values and other available information such as the model input values, one could apply their selection method to select any number of features that meet the criteria. For example, one may select all features with positive contribution to the undesirable outcome prediction.

Finally, the interpretation for each model would be based on the model input feature. For instance, each layer 1 pathway model would suggest potential gene targets, the entire model would also suggest potential gene targets, while the layer 2 meta model would suggest which layer 1 model outputs are contributing most to the final risk score.

Counterfactual explanations determine which minimal changes to feature values would alter the model's prediction for a given sample. This helps identify which features have the most influence on changing an outcome. For every model in the Ensemble Classifier, local explanations can be obtained using any algorithms based on Counterfactual Explanations such as DiCE or LORE.

To calculate local explanation for n samples with m features, one should prepare a trained model, sufficient or all samples from the training matrix with their corresponding labels, and the matrix of interest of dimension n×m. Then provide them to a compatible explainer with the appropriate arguments.

In the case of binary classification, this should obtain a n×m matrix of local explanations, which are the calculated or approximated contributions of each feature for each sample towards predicting one of the two classes.

Based on the calculated values and other available information such as the model input values, one could apply their selection method to select any number of features that meet the criteria. For example, one may select all features with positive contribution to the undesirable outcome prediction. Finally, the interpretation for each model would be based on the model input feature. For instance, each layer 1 pathway model would suggest potential gene targets, the entire model would also suggest potential gene targets, while the layer 2 meta model would suggest which layer 1 model output are contributing most to the final risk score.

Anchors find feature conditions that guarantee a consistent model prediction. Unlike other methods that provide importance scores, Anchors generate rule-based explanations that define under what conditions a prediction remains unchanged.

For every model in the Ensemble Classifier, local explanations can be obtained using Anchor explainer.

To calculate local explanation for n samples with m features, one should prepare a trained model, sufficient or all samples from the training matrix with their corresponding labels, and the matrix of interest of dimension n×m. Then provide them to a compatible explainer with the appropriate arguments.

In the case of binary classification, this should obtain a n×m matrix of local explanations, which are the calculated or approximated contributions of each feature for each sample towards predicting one of the two classes.

Based on the calculated values and other available information such as the model input values, one could apply their selection method to select any number of features that meet the criteria. For example, one may select all features with positive contribution to the undesirable outcome prediction.

Finally, the interpretation for each model would be based on the model input feature. For instance, each layer 1 pathway model would suggest potential gene targets, the entire model would also suggest potential gene targets, while the layer 2 meta model would suggest which layer 1 model output are contributing most to the final risk score.

In one embodiment, said state of interest is selected from the group consisting of breast cancer, ovarian cancer, lung cancer, colorectal cancer, small cell lung cancer, liver cancer and prostate cancer.

In one embodiment, said functional enrichment analysis is performed using publicly available online platforms to identify biological processes associated with the state of interest.

In one embodiment, said the gene expression data is obtained through RNA sequencing, microarrays, or retrieved from publicly available data repositories.

In one embodiment, the method further comprises preprocessing steps selected from the group comprising: a. quality control, transcript alignment; b. gene count quantification, normalization; and c. gene annotation prior to functional enrichment analysis.

In one embodiment, said machine learning model is: a. trained using a training dataset of gene expression data and known disease states; and b. validated using performance metrics comprising cross-validation.

In one embodiment, the method further comprises generating a recommendation for therapeutic intervention based on dysregulated pathway scores and the predicted efficacy of available drugs or treatments for the pathway, wherein said therapeutic intervention is selected from the group comprising: a. small molecule drugs; b. biologics; c. gene therapies; d. cell-based therapies; e. immunotherapies; f. combination therapies; g. targeted radiotherapies; h. dietary or lifestyle interventions; and i. alternative therapeutic options.

In one embodiment, the method further comprises: a. validating treatment efficacy by comparing pre-treatment and post-treatment dysregulated pathway scores; and b. generating an adjusted treatment recommendation if a subject's dysregulated pathway score changes.

In one embodiment, the method further comprises: a. generating a personalized treatment recommendation based on state of interest severity score; b. generating a recommendation for the administration of the personalized treatment based on state of interest severity score; and c. ranking patients for prioritized personalized treatment based on state of interest severity score.

In one embodiment, the method further comprises: a. monitoring longitudinal changes in a subject's state of interest severity scores; and b, generating an adjusted treatment recommendation if the subject's state of interest severity scores score changes.

In one embodiment, the method further comprises detecting molecular targets for personalized treatment using the values indicating the impact of each gene on the state-specific pathway's contribution to the final state of interest classification.

In one embodiment, the values indicating the impact of each gene on the state-specific pathway's contribution to the final state of interest classification are Shapley Additive Explanations values.

In one embodiment, the Shapley Additive Explanations values provide global interpretability by identifying genes that influence state of interest classification across the entire dataset, and local interpretability by providing a detailed breakdown of gene-level contributions for each individual sample.

In one embodiment, the machine learning model and Shapley Additive Explanations generating steps are subject-independent, allowing for the generation of personalized treatment strategies for an individual subject based on gene expression data.

In one embodiment, the invention is a personalized treatment method for a state of interest, the method comprising: a. receiving a first gene expression dataset from cells in a state of interest; b. receiving a second gene expression dataset from cells in a reference state: c. detecting dysregulated gene sets related to the state of interest using whole-genome co-expression network analysis and differential gene expression analysis; d. generating state-specific pathways using functional enrichment analysis on said dysregulated gene sets; e. generating a dysregulated pathway score for each state-specific pathway using a machine learning model comprising a two-layer ensemble approach, wherein: i. a first layer predicts states of interest based on the state-specific pathways using classifiers selected based on optimal performance metrics; ii. each state-specific pathway is associated with a state of interest severity probability in the first layer; iii. a second layer integrates probabilities from the first layer and computes a final state of interest classification using a stacking classifier; iv. the severity probability of each state-specific pathway is used to assign a weight to that state-specific pathway in the final classification; and v. the weight of each state-specific pathway is multiplied by that state-specific pathway's probability, generating a dysregulated pathway score for each state-specific pathway; f. scaling and normalizing said dysregulated pathway scores, wherein higher scores indicate a greater likelihood of contribution to the state of interest; g. generating values indicating the impact of each gene on the state-specific pathway's contribution to the final state of interest classification at the model-wide and sample-specific levels; h. detecting molecular targets for personalized treatment using the values indicating the impact of each gene on the state-specific pathway's contribution to the final state of interest classification; i. generating a recommended personalized treatment; and j. administering the personalized treatment.

In one embodiment, the invention is a system for analyzing biological pathways associated with a state of interest, the system comprising: a. a processor; b. memory; and c. program instructions, stored in the memory, that upon execution by the processor cause the computing device to perform operations for analyzing biological pathways associated with a state of interest, said operations comprising the steps of: i. receiving a first gene expression dataset from cells in a state of interest; ii. receiving a second gene expression dataset from cells in a reference state; iii. detecting dysregulated gene sets related to the state of interest using whole-genome co-expression network analysis and differential gene expression analysis: iv. generating state-specific pathways using functional enrichment analysis on said dysregulated gene sets; v. generating a dysregulated pathway score for each state-specific pathway using a machine learning model comprising a two-layer ensemble approach, wherein: 1. a first layer predicts states of interest based on the state-specific pathways using classifiers selected based on optimal performance metrics; 2. each state-specific pathway is associated with a state of interest severity probability in the first layer; 3. a second layer integrates probabilities from the first layer and computes a final state of interest classification using a stacking classifier; 4. the severity probability of each state-specific pathway is used to assign a weight to that state-specific pathway in the final classification; and 5. the weight of each state-specific pathway is multiplied by that state-specific pathway's probability, generating a dysregulated pathway score for each state-specific pathway; vi. scaling and normalizing said dysregulated pathway scores, wherein higher scores indicate a greater likelihood of contribution to the state of interest; and vii. generating values indicating the impact of each gene on the state-specific pathway's contribution to the final state of interest classification at the model-wide and sample-specific levels.

In one embodiment, said operations further comprise the step of detecting molecular targets for personalized treatment using the values indicating the impact of each gene on the state-specific pathway's contribution to the final state of interest classification.

In one embodiment, the system further comprises generating a recommended personalized treatment.

In one embodiment, the system further comprises administering a recommended personalized treatment.

In one embodiment, the values indicating the impact of each gene on the state-specific pathway's contribution to the final state of interest classification are Shapley Additive Explanations values.

In one embodiment, the values indicating the impact of each gene on the state-specific pathway's contribution to the final state of interest classification are Shapley Additive Explanations values.

In one embodiment, the state of interest is ovarian cancer.

In one embodiment, the cells are human cells.

### Example 1 Analysis Pipeline

Publicly accessible raw human transcriptomic datasets are acquired from online repositories. The keywords and selection criteria for these datasets are specifically tailored to the disease under investigation. Following acquisition, the datasets undergo preprocessing in accordance with one's internal pipeline, which includes, but is not limited to, quality control, transcript alignment, gene count quantification, normalization, and gene annotation.

Given the utilization of multiple datasets and the inherent variability among them, one may first consolidate all datasets that meet one's inclusion criteria and subsequently apply batch correction using established methods in the field.

The batch-corrected assembled dataset is then subjected to whole-genome co-expression network analysis and differential gene expression analysis to identify dysregulated gene sets of biological significance related to the disease. These gene sets are submitted to the online platform gProfiler for functional (pathway) enrichment analysis. The results generated will constitute one's disease-specific database for input into the machine learning model.

The machine learning model (MLM) employs an ensemble approach comprising two layers, with the output of the first layer serving as input for the second layer. In the first layer, pathways derived from the functional enrichment analysis are utilized in various machine learning classifiers to predict disease states. The classifier for each pathway is selected based on optimal performance metrics, such as the area under the curve (AUC). Each pathway is associated with its own disease severity probability, which is subsequently used as input for the second layer of the MLM, consisting of a stacking machine learning classifier. This classifier integrates the probabilities from all first-layer models to ascertain the final disease state of the human tissue sample under analysis. In this layer, different weights are assigned to each pathway based on its severity probability, which are then multiplied by the probabilities obtained from the first layer, producing a score for each pathway. This score is normalized and adjusted to ensure it falls within a range of 0 to 100, with higher scores reflecting a greater likelihood of contribution to the disease. All pathways are then subjected to further analysis to identify critical molecular targets for targeted treatment using the Shapley Additive Explanations (SHAP) method.

The SHAP method enhances the interpretability of the model's predictions by integrating SHAP values, which provide insights into feature contributions at both global (model-wide) and local (sample-specific) levels. Based on cooperative game theory, SHAP calculates the contribution of each gene expression feature to the prediction, attributing values that indicate the positive or negative impact of each gene on the model's decision regarding disease classification. Global Interpretability: SHAP summaries are employed to identify key genes that consistently influence classification decisions across the entire dataset. By aggregating SHAP values, the model identifies prominent biomarkers associated with, for instance, tumorigenic processes, such as the overexpression or under expression of specific oncogenes or tumor suppressor genes. These insights can guide biological interpretation and suggest potential gene targets for further research. Local Interpretability: For each individual sample, SHAP values provide a detailed breakdown of gene-level contributions to the predicted disease classification. For instance, in a sample predicted as cancerous, the SHAP explanation may highlight a set of upregulated oncogenes or downregulated tumor suppressors that drive the classification outcome. SHAP calculations are performed for every model in the stacking model, encompassing both the first and second layers, to obtain explanations for each model. Genes identified as having a positive contribution to predicting the disease state in instances of overexpression may serve as potential candidate targets for selecting appropriate targeted therapeutic drugs.

### Example 2 Chemoresistance of High Grade Serous Ovarian Cancer

In one embodiment, this invention provides a method for predicting chemotherapy resistance in high-grade serous ovarian cancer (HGSOC). Tumor tissue samples are obtained, and RNA sequencing is conducted using Next Generation Sequencing (NGS). The resulting RNA sequencing data undergo preprocessing before being input into the machine learning model (MLM) for the identification of chemotherapy resistance risk and prioritization of dysregulated pathways. Each pathway within the established MLM is assigned a dysregulated pathway (DP) score, calculated as the first-layer probability multiplied by the second-layer weights. Pathways with the highest DP scores are considered the most significant contributors to resistance risk. The Shapley Additive Explanations (SHAP) method is employed to identify the genes that most significantly influence the high-probability predictions. The gene with the highest positive contribution to predicting resistance is then matched with an appropriate FDA-approved targeted therapy.

TABLE 1
Biological pathways and their dysregulated pathway (DP) scores.
The DP scores vary for different pathways across each patient,
enabling personalized treatment strategies.

| Biological | Pa- | Pa- | Pa- | Pa- | Pa- | Pa- |
|---|---|---|---|---|---|---|
| Pathways | tient | tient | tient | tient | tient | tient |
| (GO ID) | 1 | 2 | 3 | 4 | 5 | 6 |
| response to cytokine | 0.63 | 0.56 | 0.62 | 0.56 | 0.88 | 0.87 |
| (GO:0034097) | | | | | | |
| ovulation cycle | 0.99 | 0.50 | 0.53 | 0.59 | 0.96 | 0.54 |
| process | | | | | | |
| (GO:0022602) | | | | | | |
| cellular response to | 0.64 | 0.56 | 0.56 | 0.67 | 0.87 | 0.81 |
| chemical stimulus | | | | | | |
| (GO:0070887) | | | | | | |
| negative regulation | 0.58 | 0.50 | 0.70 | 0.67 | 0.81 | 0.73 |
| of multicellular | | | | | | |
| organismal process | | | | | | |
| (GO:0051241) | | | | | | |
| cellular response to | 0.54 | 0.59 | 0.50 | 0.59 | 0.86 | 0.87 |
| cytokine stimulus | | | | | | |
| (GO:0071345) | | | | | | |
| protein modification | 0.49 | 0.69 | 0.81 | 0.85 | 0.57 | 0.48 |
| by small protein | | | | | | |
| conjugation | | | | | | |
| (GO:0032446) | | | | | | |
| protein metabolic | 0.52 | 0.56 | 0.57 | 0.60 | 0.71 | 0.81 |
| process | | | | | | |
| (GO:0019538) | | | | | | |
| embryo | 0.64 | 0.56 | 0.68 | 0.62 | 0.68 | 0.57 |
| development | | | | | | |
| (GO:0009790) | | | | | | |
| multicellular | 0.51 | 0.55 | 0.71 | 0.57 | 0.72 | 0.69 |
| organismal process | | | | | | |
| (GO:0032501) | | | | | | |
| intracellular protein | 0.48 | 0.48 | 0.79 | 0.48 | 0.79 | 0.74 |
| transport | | | | | | |
| (GO:0006886) | | | | | | |
| regulation of protein | 0.52 | 0.74 | 0.74 | 0.52 | 0.72 | 0.48 |
| localization to | | | | | | |
| plasma membrane | | | | | | |
| (GO:1903076) | | | | | | |
| positive regulation | 0.52 | 0.56 | 0.70 | 0.72 | 0.69 | 0.50 |
| of chromosome | | | | | | |
| separation | | | | | | |
| (GO:1905820) | | | | | | |
| positive regulation | 0.69 | 0.52 | 0.56 | 0.70 | 0.68 | 0.52 |
| of cellular | | | | | | |
| component | | | | | | |
| organization | | | | | | |
| (GO:0051130) | | | | | | |
| negative regulation | 0.64 | 0.53 | 0.50 | 0.67 | 0.68 | 0.60 |
| of BMP signaling | | | | | | |
| pathway | | | | | | |
| (GO:0030514) | | | | | | |
| response to oxygen | 0.66 | 0.64 | 0.59 | 0.49 | 0.74 | 0.50 |
| levels | | | | | | |
| (GO:0070482) | | | | | | |
| developmental | 0.62 | 0.53 | 0.52 | 0.53 | 0.65 | 0.74 |
| growth involved in | | | | | | |
| morphogenesis | | | | | | |
| (GO:0060560) | | | | | | |
| proteolysis | 0.53 | 0.53 | 0.63 | 0.61 | 0.62 | 0.65 |
| (GO:0006508) | | | | | | |
| macromolecule | 0.48 | 0.76 | 0.77 | 0.50 | 0.56 | 0.50 |
| localization | | | | | | |
| (GO:0033036) | | | | | | |
| proteolysis involved | 0.48 | 0.59 | 0.71 | 0.48 | 0.72 | 0.58 |
| in protein catabolic | | | | | | |
| process | | | | | | |
| (GO:0051603) | | | | | | |
| sensory organ | 0.63 | 0.52 | 0.61 | 0.59 | 0.55 | 0.65 |
| development | | | | | | |
| (GO:0007423) | | | | | | |
| vesicle-mediated | 0.66 | 0.64 | 0.64 | 0.48 | 0.63 | 0.49 |
| transport in synapse | | | | | | |
| (GO:0099003) | | | | | | |
| endothelial cell | 0.56 | 0.50 | 0.72 | 0.50 | 0.70 | 0.54 |
| apoptotic process | | | | | | |
| (GO:0072577) | | | | | | |
| positive regulation | 0.83 | 0.48 | 0.48 | 0.78 | 0.48 | 0.48 |
| of MAPK cascade | | | | | | |
| (GO:0043410) | | | | | | |
| regulation of | 0.50 | 0.49 | 0.66 | 0.71 | 0.66 | 0.48 |
| localization | | | | | | |
| (GO:0032879) | | | | | | |
| tissue migration | 0.72 | 0.48 | 0.51 | 0.48 | 0.56 | 0.71 |
| (GO:0090130) | | | | | | |
| regulation of | 0.48 | 0.64 | 0.71 | 0.48 | 0.52 | 0.64 |
| biological process | | | | | | |
| (GO:0050789) | | | | | | |
| positive regulation | 0.49 | 0.51 | 0.62 | 0.60 | 0.60 | 0.62 |
| of transcription by | | | | | | |
| RNA polymerase II | | | | | | |
| (GO:0045944) | | | | | | |
| organonitrogen | 0.53 | 0.55 | 0.56 | 0.57 | 0.60 | 0.64 |
| compound metabolic | | | | | | |
| process | | | | | | |
| (GO:1901564) | | | | | | |
| organelle | 0.56 | 0.48 | 0.70 | 0.52 | 0.65 | 0.54 |
| organization | | | | | | |
| (GO:0006996) | | | | | | |
| modification- | 0.61 | 0.64 | 0.48 | 0.48 | 0.66 | 0.57 |
| dependent protein | | | | | | |
| catabolic process | | | | | | |
| (GO:0019941) | | | | | | |
| positive regulation | 0.64 | 0.53 | 0.51 | 0.56 | 0.61 | 0.58 |
| of multicellular | | | | | | |
| organismal process | | | | | | |
| (GO:0051240) | | | | | | |
| collagen fibril | 0.48 | 0.55 | 0.66 | 0.60 | 0.62 | 0.49 |
| organization | | | | | | |
| (GO:0030199) | | | | | | |
| synaptic vesicle | 0.62 | 0.56 | 0.61 | 0.56 | 0.51 | 0.53 |
| endocytosis | | | | | | |
| (GO:0048488) | | | | | | |
| intracellular | 0.48 | 0.49 | 0.63 | 0.53 | 0.66 | 0.59 |
| transport | | | | | | |
| (GO:0046907) | | | | | | |
| multicellular | 0.48 | 0.53 | 0.73 | 0.48 | 0.70 | 0.48 |
| organismal process | | | | | | |
| (GO:0032501) | | | | | | |
| regulation of | 0.60 | 0.49 | 0.52 | 0.53 | 0.61 | 0.62 |
| locomotion | | | | | | |
| (GO:0040012) | | | | | | |
| organonitrogen | 0.54 | 0.51 | 0.62 | 0.54 | 0.61 | 0.53 |
| compound metabolic | | | | | | |
| process | | | | | | |
| (GO:1901564) | | | | | | |
| regulation of cell | 0.62 | 0.53 | 0.53 | 0.52 | 0.57 | 0.59 |
| motility | | | | | | |
| (GO:2000145) | | | | | | |
| ear development | 0.57 | 0.52 | 0.63 | 0.53 | 0.54 | 0.57 |
| (GO:0043583) | | | | | | |
| cell division | 0.53 | 0.55 | 0.57 | 0.58 | 0.59 | 0.53 |
| (GO:0051301) | | | | | | |
| negative regulation | 0.48 | 0.59 | 0.52 | 0.52 | 0.62 | 0.61 |
| of cell adhesion | | | | | | |
| (GO:0007162) | | | | | | |
| positive regulation | 0.53 | 0.53 | 0.56 | 0.56 | 0.59 | 0.57 |
| of apoptotic process | | | | | | |
| (GO:0043065) | | | | | | |
| collagen metabolic | 0.51 | 0.53 | 0.51 | 0.55 | 0.64 | 0.58 |
| process | | | | | | |
| (GO:0032963) | | | | | | |
| modification- | 0.49 | 0.62 | 0.52 | 0.48 | 0.63 | 0.57 |
| dependent | | | | | | |
| macromolecule | | | | | | |
| catabolic process | | | | | | |
| (GO:0043632) | | | | | | |
| regulation of cellular | 0.54 | 0.49 | 0.54 | 0.51 | 0.62 | 0.59 |
| metabolic process | | | | | | |
| (GO:0031323) | | | | | | |
| ameboidal-type cell | 0.58 | 0.51 | 0.54 | 0.52 | 0.54 | 0.60 |
| migration | | | | | | |
| (GO:0001667) | | | | | | |
| kidney epithelium | 0.52 | 0.49 | 0.52 | 0.52 | 0.60 | 0.64 |
| development | | | | | | |
| (GO:0072073) | | | | | | |
| protein localization | 0.48 | 0.51 | 0.62 | 0.48 | 0.58 | 0.60 |
| (GO:0008104) | | | | | | |
| cell migration | 0.54 | 0.53 | 0.54 | 0.50 | 0.61 | 0.55 |
| (GO:0016477) | | | | | | |
| regulation of cell | 0.58 | 0.53 | 0.53 | 0.53 | 0.54 | 0.56 |
| development | | | | | | |
| (GO:0060284) | | | | | | |
| regulation of cellular | 0.53 | 0.49 | 0.55 | 0.52 | 0.61 | 0.58 |
| process | | | | | | |
| (GO:0050794) | | | | | | |
| anatomical structure | 0.58 | 0.48 | 0.58 | 0.48 | 0.58 | 0.58 |
| morphogenesis | | | | | | |
| (GO:0009653) | | | | | | |
| enzyme-linked | 0.54 | 0.51 | 0.52 | 0.52 | 0.61 | 0.58 |
| receptor protein | | | | | | |
| signaling pathway | | | | | | |
| (GO:0007167) | | | | | | |
| endothelial cell | 0.56 | 0.49 | 0.54 | 0.52 | 0.59 | 0.58 |
| migration | | | | | | |
| (GO:0043542) | | | | | | |
| regulation of cell | 0.56 | 0.51 | 0.55 | 0.53 | 0.56 | 0.55 |
| population | | | | | | |
| proliferation | | | | | | |
| (GO:0042127) | | | | | | |
| heterocycle | 0.54 | 0.51 | 0.57 | 0.54 | 0.58 | 0.52 |
| biosynthetic process | | | | | | |
| (GO:0018130) | | | | | | |
| epithelial cell | 0.61 | 0.49 | 0.51 | 0.48 | 0.55 | 0.60 |
| migration | | | | | | |
| (GO:0010631) | | | | | | |
| cell population | 0.56 | 0.51 | 0.54 | 0.53 | 0.54 | 0.57 |
| proliferation | | | | | | |
| (GO:0008283) | | | | | | |
| negative regulation | 0.48 | 0.51 | 0.53 | 0.52 | 0.67 | 0.54 |
| of programmed cell | | | | | | |
| death (GO:0043069) | | | | | | |
| positive regulation | 0.56 | 0.52 | 0.53 | 0.53 | 0.55 | 0.55 |
| of cell population | | | | | | |
| proliferation | | | | | | |
| (GO:0008284) | | | | | | |
| positive regulation | 0.48 | 0.54 | 0.72 | 0.48 | 0.51 | 0.53 |
| of biological process | | | | | | |
| (GO:0048518) | | | | | | |
| regulation of | 0.54 | 0.52 | 0.53 | 0.53 | 0.58 | 0.54 |
| molecular function | | | | | | |
| (GO:0065009) | | | | | | |
| regulation of | 0.53 | 0.49 | 0.53 | 0.51 | 0.60 | 0.58 |
| macromolecule | | | | | | |
| metabolic process | | | | | | |
| (GO:0060255) | | | | | | |
| negative regulation | 0.53 | 0.52 | 0.51 | 0.56 | 0.57 | 0.55 |
| of transmembrane | | | | | | |
| receptor protein | | | | | | |
| serine/threonine | | | | | | |
| kinase signaling | | | | | | |
| pathway | | | | | | |
| (GO:0090101) | | | | | | |
| regulation of | 0.52 | 0.49 | 0.54 | 0.51 | 0.59 | 0.57 |
| biological process | | | | | | |
| (GO:0050789) | | | | | | |
| phosphate- | 0.58 | 0.48 | 0.57 | 0.48 | 0.60 | 0.54 |
| containing | | | | | | |
| compound metabolic | | | | | | |
| process | | | | | | |
| (GO:0006796) | | | | | | |
| regulation of | 0.53 | 0.49 | 0.53 | 0.51 | 0.60 | 0.58 |
| metabolic process | | | | | | |
| (GO:0019222) | | | | | | |
| anterior/posterior | 0.48 | 0.48 | 0.65 | 0.48 | 0.65 | 0.48 |
| pattern specification | | | | | | |
| (GO:0009952) | | | | | | |
| signaling | 0.48 | 0.48 | 0.58 | 0.51 | 0.62 | 0.54 |
| (GO:0023052) | | | | | | |
| generation of | 0.54 | 0.53 | 0.54 | 0.50 | 0.55 | 0.55 |
| neurons | | | | | | |
| (GO:0048699) | | | | | | |
| post-translational | 0.48 | 0.50 | 0.75 | 0.51 | 0.49 | 0.48 |
| protein modification | | | | | | |
| (GO:0043687) | | | | | | |
| regulation of | 0.52 | 0.50 | 0.54 | 0.55 | 0.55 | 0.55 |
| supramolecular fiber | | | | | | |
| organization | | | | | | |
| (GO:1902903) | | | | | | |
| regulation of | 0.53 | 0.49 | 0.53 | 0.51 | 0.58 | 0.56 |
| primary metabolic | | | | | | |
| process | | | | | | |
| (GO:0080090) | | | | | | |
| positive regulation | 0.56 | 0.48 | 0.58 | 0.51 | 0.59 | 0.48 |
| of nitrogen | | | | | | |
| compound metabolic | | | | | | |
| process | | | | | | |
| (GO:0051173) | | | | | | |
| biological regulation | 0.52 | 0.49 | 0.53 | 0.51 | 0.58 | 0.56 |
| (GO:0065007) | | | | | | |
| regulation of | 0.53 | 0.49 | 0.52 | 0.50 | 0.58 | 0.56 |
| nitrogen compound | | | | | | |
| metabolic process | | | | | | |
| (GO:0051171) | | | | | | |
| regulation of bone | 0.51 | 0.50 | 0.54 | 0.54 | 0.56 | 0.54 |
| mineralization | | | | | | |
| (GO:0030500) | | | | | | |
| cell differentiation | 0.48 | 0.48 | 0.60 | 0.55 | 0.48 | 0.60 |
| (GO:0030154) | | | | | | |
| organic substance | 0.53 | 0.50 | 0.59 | 0.48 | 0.54 | 0.54 |
| transport | | | | | | |
| (GO:0071702) | | | | | | |
| regulation of cellular | 0.58 | 0.50 | 0.49 | 0.54 | 0.56 | 0.52 |
| component size | | | | | | |
| (GO:0032535) | | | | | | |
| heterocycle | 0.53 | 0.49 | 0.54 | 0.48 | 0.58 | 0.56 |
| biosynthetic process | | | | | | |
| (GO:0018130) | | | | | | |
| regulation of | 0.51 | 0.50 | 0.53 | 0.53 | 0.56 | 0.54 |
| biomineral tissue | | | | | | |
| development | | | | | | |
| (GO:0070167) | | | | | | |
| negative regulation | 0.51 | 0.50 | 0.53 | 0.51 | 0.58 | 0.55 |
| of nucleobase- | | | | | | |
| containing | | | | | | |
| compound metabolic | | | | | | |
| process | | | | | | |
| (GO:0045934) | | | | | | |
| organonitrogen | 0.55 | 0.48 | 0.69 | 0.48 | 0.48 | 0.48 |
| compound metabolic | | | | | | |
| process | | | | | | |
| (GO:1901564) | | | | | | |
| regulation of | 0.52 | 0.50 | 0.53 | 0.52 | 0.57 | 0.54 |
| biological quality | | | | | | |
| (GO:0065008) | | | | | | |
| cellular localization | 0.53 | 0.48 | 0.59 | 0.48 | 0.54 | 0.55 |
| (GO:0051641) | | | | | | |
| regulation of | 0.56 | 0.49 | 0.50 | 0.51 | 0.56 | 0.55 |
| anatomical structure | | | | | | |
| morphogenesis | | | | | | |
| (GO:0022603) | | | | | | |
| neuron | 0.54 | 0.53 | 0.52 | 0.50 | 0.54 | 0.53 |
| differentiation | | | | | | |
| (GO:0030182) | | | | | | |
| regulation of | 0.52 | 0.51 | 0.53 | 0.52 | 0.54 | 0.52 |
| signaling | | | | | | |
| (GO:0023051) | | | | | | |
| regulation of | 0.53 | 0.53 | 0.59 | 0.48 | 0.54 | 0.48 |
| transport | | | | | | |
| (GO:0051049) | | | | | | |
| negative regulation | 0.50 | 0.51 | 0.54 | 0.55 | 0.52 | 0.53 |
| of cell cycle phase | | | | | | |
| transition | | | | | | |
| (GO:1901988) | | | | | | |
| response to stimulus | 0.52 | 0.49 | 0.51 | 0.51 | 0.57 | 0.55 |
| (GO:0050896) | | | | | | |
| reproductive | 0.53 | 0.53 | 0.52 | 0.51 | 0.54 | 0.52 |
| structure | | | | | | |
| development | | | | | | |
| (GO:0048608) | | | | | | |
| positive regulation | 0.52 | 0.51 | 0.49 | 0.50 | 0.58 | 0.55 |
| of macromolecule | | | | | | |
| metabolic process | | | | | | |
| (GO:0010604) | | | | | | |
| positive regulation | 0.50 | 0.52 | 0.52 | 0.54 | 0.55 | 0.52 |
| of DNA-templated | | | | | | |
| transcription | | | | | | |
| (GO:0045893) | | | | | | |
| cardiac chamber | 0.52 | 0.54 | 0.51 | 0.51 | 0.55 | 0.52 |
| morphogenesis | | | | | | |
| (GO:0003206) | | | | | | |
| cell motility | 0.52 | 0.50 | 0.53 | 0.50 | 0.57 | 0.52 |
| (GO:0048870) | | | | | | |
| positive regulation | 0.58 | 0.49 | 0.57 | 0.48 | 0.53 | 0.50 |
| of metabolic process | | | | | | |
| (GO:0009893) | | | | | | |
| negative regulation | 0.51 | 0.49 | 0.52 | 0.51 | 0.57 | 0.54 |
| of nitrogen | | | | | | |
| compound metabolic | | | | | | |
| process | | | | | | |
| (GO:0051172) | | | | | | |
| negative regulation | 0.50 | 0.49 | 0.53 | 0.51 | 0.58 | 0.54 |
| of cellular process | | | | | | |
| (GO:0048523) | | | | | | |
| establishment of | 0.49 | 0.49 | 0.60 | 0.49 | 0.52 | 0.56 |
| protein localization | | | | | | |
| (GO:0045184) | | | | | | |
| proteasomal protein | 0.48 | 0.48 | 0.56 | 0.48 | 0.62 | 0.53 |
| catabolic process | | | | | | |
| (GO:0010498) | | | | | | |
| cellular component | 0.51 | 0.49 | 0.52 | 0.51 | 0.56 | 0.54 |
| organization | | | | | | |
| (GO:0016043) | | | | | | |
| negative regulation | 0.52 | 0.48 | 0.51 | 0.48 | 0.57 | 0.58 |
| of nitrogen | | | | | | |
| compound metabolic | | | | | | |
| process | | | | | | |
| (GO:0051172) | | | | | | |
| regulation of protein | 0.53 | 0.49 | 0.58 | 0.50 | 0.52 | 0.50 |
| metabolic process | | | | | | |
| (GO:0051246) | | | | | | |
| regulation of | 0.50 | 0.49 | 0.52 | 0.51 | 0.55 | 0.53 |
| localization | | | | | | |
| (GO:0032879) | | | | | | |
| cellular response to | 0.51 | 0.49 | 0.51 | 0.51 | 0.56 | 0.54 |
| stimulus | | | | | | |
| (GO:0051716) | | | | | | |
| response to chemical | 0.51 | 0.49 | 0.50 | 0.51 | 0.56 | 0.54 |
| (GO:0042221) | | | | | | |
| regulation of | 0.53 | 0.50 | 0.50 | 0.50 | 0.55 | 0.53 |
| transferase activity | | | | | | |
| (GO:0051338) | | | | | | |
| positive regulation | 0.51 | 0.49 | 0.51 | 0.53 | 0.53 | 0.54 |
| of cellular | | | | | | |
| component | | | | | | |
| biogenesis | | | | | | |
| (GO:0044089) | | | | | | |
| cellular response to | 0.50 | 0.49 | 0.51 | 0.51 | 0.56 | 0.54 |
| organic substance | | | | | | |
| (GO:0071310) | | | | | | |
| inner ear | 0.52 | 0.52 | 0.52 | 0.51 | 0.51 | 0.53 |
| development | | | | | | |
| (GO:0048839) | | | | | | |
| regulation of | 0.51 | 0.49 | 0.53 | 0.51 | 0.54 | 0.52 |
| cytoskeleton | | | | | | |
| organization | | | | | | |
| (GO:0051493) | | | | | | |
| negative regulation | 0.50 | 0.49 | 0.52 | 0.50 | 0.55 | 0.53 |
| of biological process | | | | | | |
| (GO:0048519) | | | | | | |
| pattern specification | 0.50 | 0.49 | 0.54 | 0.49 | 0.55 | 0.52 |
| process | | | | | | |
| (GO:0007389) | | | | | | |
| signaling | 0.51 | 0.49 | 0.51 | 0.50 | 0.55 | 0.53 |
| (GO:0023052) | | | | | | |
| response to organic | 0.51 | 0.49 | 0.50 | 0.51 | 0.55 | 0.53 |
| substance | | | | | | |
| (GO:0010033) | | | | | | |
| regulation of cellular | 0.50 | 0.49 | 0.52 | 0.51 | 0.54 | 0.53 |
| component | | | | | | |
| biogenesis | | | | | | |
| (GO:0044087) | | | | | | |
| regulation of | 0.52 | 0.50 | 0.53 | 0.50 | 0.51 | 0.52 |
| response to stimulus | | | | | | |
| (GO:0048583) | | | | | | |
| positive regulation | 0.54 | 0.50 | 0.57 | 0.48 | 0.50 | 0.49 |
| of signal | | | | | | |
| transduction | | | | | | |
| (GO:0009967) | | | | | | |
| organelle | 0.48 | 0.48 | 0.54 | 0.48 | 0.60 | 0.50 |
| localization | | | | | | |
| (GO:0051640) | | | | | | |
| response to oxygen- | 0.51 | 0.49 | 0.50 | 0.51 | 0.54 | 0.52 |
| containing | | | | | | |
| compound | | | | | | |
| (GO:1901700) | | | | | | |
| negative regulation | 0.51 | 0.51 | 0.51 | 0.51 | 0.52 | 0.51 |
| of canonical Wnt | | | | | | |
| signaling pathway | | | | | | |
| (GO:0090090) | | | | | | |
| intracellular signal | 0.51 | 0.49 | 0.51 | 0.50 | 0.54 | 0.52 |
| transduction | | | | | | |
| (GO:0035556) | | | | | | |
| membrane | 0.50 | 0.53 | 0.52 | 0.50 | 0.51 | 0.51 |
| organization | | | | | | |
| (GO:0061024) | | | | | | |
| developmental | 0.51 | 0.49 | 0.51 | 0.50 | 0.54 | 0.52 |
| process | | | | | | |
| (GO:0032502) | | | | | | |
| signal transduction | 0.51 | 0.49 | 0.51 | 0.50 | 0.54 | 0.52 |
| (GO:0007165) | | | | | | |
| regulation of | 0.56 | 0.48 | 0.49 | 0.48 | 0.50 | 0.56 |
| developmental | | | | | | |
| process | | | | | | |
| (GO:0050793) | | | | | | |
| organic cyclic | 0.53 | 0.50 | 0.51 | 0.48 | 0.49 | 0.56 |
| compound | | | | | | |
| biosynthetic process | | | | | | |
| (GO:1901362) | | | | | | |
| positive regulation | 0.50 | 0.48 | 0.52 | 0.53 | 0.53 | 0.50 |
| of signal | | | | | | |
| transduction | | | | | | |
| (GO:0009967) | | | | | | |
| negative regulation | 0.50 | 0.50 | 0.50 | 0.52 | 0.53 | 0.51 |
| of cell | | | | | | |
| differentiation | | | | | | |
| (GO:0045596) | | | | | | |
| regulation of | 0.50 | 0.49 | 0.51 | 0.50 | 0.54 | 0.52 |
| signaling | | | | | | |
| (GO:0023051) | | | | | | |
| negative regulation | 0.52 | 0.51 | 0.50 | 0.50 | 0.51 | 0.51 |
| of TORC1 signaling | | | | | | |
| (GO:1904262) | | | | | | |
| tissue | 0.49 | 0.49 | 0.51 | 0.51 | 0.53 | 0.52 |
| morphogenesis | | | | | | |
| (GO:0048729) | | | | | | |
| cell junction | 0.50 | 0.49 | 0.50 | 0.51 | 0.53 | 0.52 |
| organization | | | | | | |
| (GO:0034330) | | | | | | |
| positive regulation | 0.55 | 0.50 | 0.48 | 0.49 | 0.54 | 0.48 |
| of cellular metabolic | | | | | | |
| process | | | | | | |
| (GO:0031325) | | | | | | |
| nitrogen compound | 0.48 | 0.49 | 0.57 | 0.48 | 0.48 | 0.57 |
| transport | | | | | | |
| (GO:0071705) | | | | | | |
| cell-cell signaling | 0.50 | 0.49 | 0.51 | 0.50 | 0.53 | 0.52 |
| (GO:0007267) | | | | | | |
| developmental | 0.48 | 0.48 | 0.54 | 0.48 | 0.54 | 0.54 |
| process | | | | | | |
| (GO:0032502) | | | | | | |
| regulation of cellular | 0.50 | 0.49 | 0.51 | 0.50 | 0.53 | 0.52 |
| component | | | | | | |
| organization | | | | | | |
| (GO:0051128) | | | | | | |
| regulation of protein | 0.50 | 0.51 | 0.53 | 0.48 | 0.52 | 0.51 |
| transport | | | | | | |
| (GO:0051223) | | | | | | |
| anatomical structure | 0.51 | 0.49 | 0.51 | 0.50 | 0.53 | 0.52 |
| development | | | | | | |
| (GO:0048856) | | | | | | |
| cell differentiation | 0.51 | 0.49 | 0.50 | 0.50 | 0.53 | 0.52 |
| (GO:0030154) | | | | | | |
| cytoskeleton | 0.50 | 0.49 | 0.51 | 0.51 | 0.53 | 0.51 |
| organization | | | | | | |
| (GO:0007010) | | | | | | |
| positive regulation | 0.50 | 0.49 | 0.51 | 0.51 | 0.52 | 0.51 |
| of macromolecule | | | | | | |
| metabolic process | | | | | | |
| (GO:0010604) | | | | | | |
| negative regulation | 0.50 | 0.51 | 0.51 | 0.50 | 0.52 | 0.50 |
| of Wnt signaling | | | | | | |
| pathway | | | | | | |
| (GO:0030178) | | | | | | |
| biological regulation | 0.49 | 0.48 | 0.53 | 0.51 | 0.49 | 0.54 |
| (GO:0065007) | | | | | | |
| positive regulation | 0.50 | 0.49 | 0.51 | 0.50 | 0.53 | 0.52 |
| of cellular process | | | | | | |
| (GO:0048522) | | | | | | |
| response to lipid | 0.50 | 0.49 | 0.50 | 0.51 | 0.52 | 0.51 |
| (GO:0033993) | | | | | | |
| locomotion | 0.51 | 0.50 | 0.50 | 0.50 | 0.52 | 0.51 |
| (GO:0040011) | | | | | | |
| supramolecular fiber | 0.50 | 0.49 | 0.51 | 0.51 | 0.52 | 0.51 |
| organization | | | | | | |
| (GO:0097435) | | | | | | |
| positive regulation | 0.50 | 0.49 | 0.51 | 0.50 | 0.53 | 0.52 |
| of cellular metabolic | | | | | | |
| process | | | | | | |
| (GO:0031325) | | | | | | |
| positive regulation | 0.50 | 0.49 | 0.50 | 0.50 | 0.53 | 0.52 |
| of metabolic process | | | | | | |
| (GO:0009893) | | | | | | |
| regulation of actin | 0.50 | 0.49 | 0.51 | 0.51 | 0.52 | 0.51 |
| cytoskeleton | | | | | | |
| organization | | | | | | |
| (GO:0032956) | | | | | | |
| positive regulation | 0.50 | 0.48 | 0.50 | 0.50 | 0.53 | 0.52 |
| of biological process | | | | | | |
| (GO:0048518) | | | | | | |
| cell development | 0.50 | 0.49 | 0.50 | 0.50 | 0.53 | 0.51 |
| (GO:0048468) | | | | | | |
| protein metabolic | 0.48 | 0.48 | 0.56 | 0.48 | 0.54 | 0.50 |
| process | | | | | | |
| (GO:0019538) | | | | | | |
| glomerulus | 0.50 | 0.50 | 0.50 | 0.51 | 0.52 | 0.51 |
| vasculature | | | | | | |
| development | | | | | | |
| (GO:0072012) | | | | | | |
| protein localization | 0.48 | 0.52 | 0.53 | 0.52 | 0.49 | 0.48 |
| to cell periphery | | | | | | |
| (GO:1990778) | | | | | | |
| regulation of | 0.49 | 0.50 | 0.52 | 0.51 | 0.51 | 0.49 |
| macromolecule | | | | | | |
| biosynthetic process | | | | | | |
| (GO:0010556) | | | | | | |
| response to nitrogen | 0.50 | 0.49 | 0.50 | 0.50 | 0.53 | 0.51 |
| compound | | | | | | |
| (GO:1901698) | | | | | | |
| intracellular | 0.50 | 0.49 | 0.50 | 0.50 | 0.52 | 0.51 |
| signaling cassette | | | | | | |
| (GO:0141124) | | | | | | |
| multicellular | 0.50 | 0.49 | 0.50 | 0.50 | 0.52 | 0.51 |
| organism | | | | | | |
| development | | | | | | |
| (GO:0007275) | | | | | | |
| negative regulation | 0.50 | 0.49 | 0.50 | 0.50 | 0.53 | 0.51 |
| of signaling | | | | | | |
| (GO:0023057) | | | | | | |
| mitotic cell cycle | 0.48 | 0.48 | 0.51 | 0.50 | 0.53 | 0.52 |
| phase transition | | | | | | |
| (GO:0044772) | | | | | | |
| vascular endothelial | 0.52 | 0.50 | 0.50 | 0.50 | 0.50 | 0.50 |
| growth factor | | | | | | |
| signaling pathway | | | | | | |
| (GO:0038084) | | | | | | |
| negative regulation | 0.50 | 0.50 | 0.49 | 0.50 | 0.51 | 0.52 |
| of locomotion | | | | | | |
| (GO:0040013) | | | | | | |
| post-embryonic eye | 0.50 | 0.49 | 0.50 | 0.50 | 0.50 | 0.51 |
| morphogenesis | | | | | | |
| (GO:0048050) | | | | | | |
| establishment of | 0.50 | 0.49 | 0.52 | 0.49 | 0.50 | 0.51 |
| localization in cell | | | | | | |
| (GO:0051649) | | | | | | |
| protein modification | 0.50 | 0.48 | 0.49 | 0.49 | 0.52 | 0.52 |
| process | | | | | | |
| (GO:0036211) | | | | | | |
| cellular response to | 0.50 | 0.49 | 0.49 | 0.50 | 0.52 | 0.51 |
| external stimulus | | | | | | |
| (GO:0071496) | | | | | | |
| regulation of | 0.49 | 0.49 | 0.51 | 0.51 | 0.51 | 0.49 |
| biosynthetic process | | | | | | |
| (GO:0009889) | | | | | | |
| negative regulation | 0.49 | 0.49 | 0.50 | 0.50 | 0.52 | 0.51 |
| of DNA-templated | | | | | | |
| transcription | | | | | | |
| (GO:0045892) | | | | | | |
| positive regulation | 0.50 | 0.48 | 0.50 | 0.49 | 0.52 | 0.51 |
| of macromolecule | | | | | | |
| metabolic process | | | | | | |
| (GO:0010604) | | | | | | |
| negative regulation | 0.49 | 0.49 | 0.50 | 0.49 | 0.52 | 0.51 |
| of RNA metabolic | | | | | | |
| process | | | | | | |
| (GO:0051253) | | | | | | |
| negative regulation | 0.50 | 0.51 | 0.49 | 0.50 | 0.51 | 0.50 |
| of cell-substrate | | | | | | |
| adhesion | | | | | | |
| (GO:0010812) | | | | | | |
| response to stress | 0.50 | 0.48 | 0.49 | 0.49 | 0.52 | 0.51 |
| (GO:0006950) | | | | | | |
| regulation of cellular | 0.49 | 0.49 | 0.51 | 0.51 | 0.51 | 0.49 |
| biosynthetic process | | | | | | |
| (GO:0031326) | | | | | | |
| regulation of | 0.50 | 0.50 | 0.50 | 0.50 | 0.51 | 0.50 |
| canonical Wnt | | | | | | |
| signaling pathway | | | | | | |
| (GO:0060828) | | | | | | |
| response to | 0.50 | 0.49 | 0.49 | 0.50 | 0.52 | 0.50 |
| organonitrogen | | | | | | |
| compound | | | | | | |
| (GO:0010243) | | | | | | |
| positive regulation | 0.50 | 0.50 | 0.50 | 0.51 | 0.51 | 0.49 |
| of RNA metabolic | | | | | | |
| process | | | | | | |
| (GO:0051254) | | | | | | |
| cellular response to | 0.50 | 0.49 | 0.50 | 0.50 | 0.52 | 0.50 |
| oxygen-containing | | | | | | |
| compound | | | | | | |
| (GO:1901701) | | | | | | |
| tissue development | 0.49 | 0.48 | 0.50 | 0.49 | 0.51 | 0.54 |
| (GO:0009888) | | | | | | |
| negative regulation | 0.49 | 0.49 | 0.51 | 0.51 | 0.50 | 0.50 |
| of cell cycle process | | | | | | |
| (GO:0010948) | | | | | | |
| regulation of | 0.49 | 0.49 | 0.51 | 0.49 | 0.50 | 0.51 |
| transport | | | | | | |
| (GO:0051049) | | | | | | |
| positive regulation | 0.50 | 0.49 | 0.50 | 0.50 | 0.50 | 0.50 |
| of cell | | | | | | |
| communication | | | | | | |
| (GO:0010647) | | | | | | |
| regulation of | 0.50 | 0.48 | 0.50 | 0.49 | 0.52 | 0.51 |
| response to stimulus | | | | | | |
| (GO:0048583) | | | | | | |
| positive regulation | 0.50 | 0.49 | 0.50 | 0.50 | 0.50 | 0.50 |
| of signaling | | | | | | |
| (GO:0023056) | | | | | | |
| cellular response to | 0.49 | 0.49 | 0.51 | 0.50 | 0.51 | 0.49 |
| stimulus | | | | | | |
| (GO:0051716) | | | | | | |
| BMP signaling | 0.49 | 0.49 | 0.49 | 0.50 | 0.51 | 0.50 |
| pathway | | | | | | |
| (GO:0030509) | | | | | | |
| response to organic | 0.49 | 0.49 | 0.49 | 0.50 | 0.51 | 0.50 |
| cyclic compound | | | | | | |
| (GO:0014070) | | | | | | |
| supramolecular fiber | 0.49 | 0.49 | 0.50 | 0.50 | 0.51 | 0.49 |
| organization | | | | | | |
| (GO:0097435) | | | | | | |
| regulation of actin | 0.49 | 0.49 | 0.50 | 0.50 | 0.51 | 0.50 |
| filament-based | | | | | | |
| process | | | | | | |
| (GO:0032970) | | | | | | |
| vesicle-mediated | 0.50 | 0.49 | 0.50 | 0.49 | 0.50 | 0.50 |
| transport | | | | | | |
| (GO:0016192) | | | | | | |
| response to BMP | 0.49 | 0.49 | 0.49 | 0.50 | 0.50 | 0.50 |
| (GO:0071772) | | | | | | |
| regulation of | 0.50 | 0.48 | 0.49 | 0.49 | 0.51 | 0.50 |
| phosphate metabolic | | | | | | |
| process | | | | | | |
| (GO:0019220) | | | | | | |
| regulation of | 0.50 | 0.48 | 0.49 | 0.49 | 0.51 | 0.51 |
| catalytic activity | | | | | | |
| (GO:0050790) | | | | | | |
| system development | 0.53 | 0.48 | 0.53 | 0.48 | 0.48 | 0.49 |
| (GO:0048731) | | | | | | |
| system development | 0.50 | 0.48 | 0.49 | 0.49 | 0.51 | 0.50 |
| (GO:0048731) | | | | | | |
| regulation of cell | 0.50 | 0.49 | 0.49 | 0.49 | 0.50 | 0.50 |
| migration | | | | | | |
| (GO:0030334) | | | | | | |
| positive regulation | 0.49 | 0.49 | 0.50 | 0.49 | 0.51 | 0.50 |
| of cell | | | | | | |
| differentiation | | | | | | |
| (GO:0045597) | | | | | | |
| catabolic process | 0.48 | 0.48 | 0.52 | 0.48 | 0.53 | 0.49 |
| (GO:0009056) | | | | | | |
| regulation of | 0.49 | 0.48 | 0.52 | 0.50 | 0.50 | 0.49 |
| biological quality | | | | | | |
| (GO:0065008) | | | | | | |
| renal system | 0.49 | 0.49 | 0.49 | 0.50 | 0.50 | 0.50 |
| vasculature | | | | | | |
| development | | | | | | |
| (GO:0061437) | | | | | | |
| regulation of cellular | 0.49 | 0.48 | 0.51 | 0.48 | 0.50 | 0.51 |
| process | | | | | | |
| (GO:0050794) | | | | | | |
| aortic valve | 0.49 | 0.49 | 0.49 | 0.49 | 0.50 | 0.50 |
| development | | | | | | |
| (GO:0003176) | | | | | | |
| endocardial cushion | 0.49 | 0.49 | 0.49 | 0.49 | 0.50 | 0.49 |
| morphogenesis | | | | | | |
| (GO:0003203) | | | | | | |
| positive regulation | 0.49 | 0.48 | 0.49 | 0.49 | 0.49 | 0.50 |
| of vasculature | | | | | | |
| development | | | | | | |
| (GO:1904018) | | | | | | |
| regulation of cell | 0.48 | 0.49 | 0.50 | 0.50 | 0.50 | 0.49 |
| cycle phase | | | | | | |
| transition | | | | | | |
| (GO:1901987) | | | | | | |
| heart valve | 0.49 | 0.49 | 0.49 | 0.49 | 0.49 | 0.49 |
| morphogenesis | | | | | | |
| (GO:0003179) | | | | | | |
| endocardial cushion | 0.49 | 0.49 | 0.49 | 0.49 | 0.49 | 0.49 |
| development | | | | | | |
| (GO:0003197) | | | | | | |
| response to external | 0.49 | 0.48 | 0.49 | 0.49 | 0.51 | 0.50 |
| stimulus | | | | | | |
| (GO:0009605) | | | | | | |
| regulation of signal | 0.50 | 0.48 | 0.52 | 0.48 | 0.51 | 0.48 |
| transduction | | | | | | |
| (GO:0009966) | | | | | | |
| positive regulation | 0.49 | 0.48 | 0.49 | 0.49 | 0.49 | 0.50 |
| of angiogenesis | | | | | | |
| (GO:0045766) | | | | | | |
| actin cytoskeleton | 0.49 | 0.48 | 0.49 | 0.49 | 0.50 | 0.50 |
| organization | | | | | | |
| (GO:0030036) | | | | | | |
| regulation of TOR | 0.49 | 0.50 | 0.49 | 0.49 | 0.49 | 0.49 |
| signaling | | | | | | |
| (GO:0032006) | | | | | | |
| regulation of mitotic | 0.48 | 0.49 | 0.49 | 0.50 | 0.50 | 0.49 |
| cell cycle phase | | | | | | |
| transition | | | | | | |
| (GO:1901990) | | | | | | |
| aortic valve | 0.49 | 0.49 | 0.49 | 0.49 | 0.50 | 0.50 |
| morphogenesis | | | | | | |
| (GO:0003180) | | | | | | |
| regulation of | 0.50 | 0.48 | 0.49 | 0.49 | 0.50 | 0.50 |
| epithelial cell | | | | | | |
| migration | | | | | | |
| (GO:0010632) | | | | | | |
| response to hormone | 0.50 | 0.49 | 0.49 | 0.49 | 0.50 | 0.49 |
| (GO:0009725) | | | | | | |
| regulation of signal | 0.49 | 0.48 | 0.49 | 0.49 | 0.50 | 0.50 |
| transduction | | | | | | |
| (GO:0009966) | | | | | | |
| positive regulation | 0.51 | 0.49 | 0.50 | 0.48 | 0.49 | 0.49 |
| of signaling | | | | | | |
| (GO:0023056) | | | | | | |
| negative regulation | 0.48 | 0.48 | 0.49 | 0.48 | 0.48 | 0.54 |
| of TOR signaling | | | | | | |
| (GO:0032007) | | | | | | |
| mesenchyme | 0.49 | 0.49 | 0.49 | 0.49 | 0.49 | 0.49 |
| morphogenesis | | | | | | |
| (GO:0072132) | | | | | | |
| heart valve | 0.49 | 0.49 | 0.49 | 0.49 | 0.49 | 0.49 |
| development | | | | | | |
| (GO:0003170) | | | | | | |
| synaptic vesicle | 0.49 | 0.49 | 0.49 | 0.49 | 0.49 | 0.49 |
| cycle (GO:0099504) | | | | | | |
| regulation of | 0.49 | 0.49 | 0.50 | 0.49 | 0.49 | 0.49 |
| response to stress | | | | | | |
| (GO:0080134) | | | | | | |
| nephron tubule | 0.49 | 0.49 | 0.49 | 0.49 | 0.49 | 0.49 |
| morphogenesis | | | | | | |
| (GO:0072078) | | | | | | |
| in utero embryonic | 0.48 | 0.49 | 0.49 | 0.50 | 0.49 | 0.49 |
| development | | | | | | |
| (GO:0001701) | | | | | | |
| regulation of BMP | 0.49 | 0.48 | 0.49 | 0.49 | 0.49 | 0.49 |
| signaling pathway | | | | | | |
| (GO:0030510) | | | | | | |
| response to | 0.49 | 0.49 | 0.49 | 0.49 | 0.49 | 0.49 |
| endogenous stimulus | | | | | | |
| (GO:0009719) | | | | | | |
| response to stimulus | 0.48 | 0.48 | 0.54 | 0.48 | 0.48 | 0.49 |
| (GO:0050896) | | | | | | |
| regulation of cell | 0.49 | 0.48 | 0.49 | 0.49 | 0.50 | 0.49 |
| differentiation | | | | | | |
| (GO:0045595) | | | | | | |
| intracellular protein | 0.49 | 0.49 | 0.49 | 0.48 | 0.49 | 0.48 |
| transport | | | | | | |
| (GO:0006886) | | | | | | |
| actin filament-based | 0.49 | 0.48 | 0.49 | 0.49 | 0.50 | 0.49 |
| process | | | | | | |
| (GO:0030029) | | | | | | |
| regulation of | 0.49 | 0.49 | 0.49 | 0.49 | 0.49 | 0.49 |
| transferase activity | | | | | | |
| (GO:0051338) | | | | | | |
| microtubule-based | 0.49 | 0.49 | 0.49 | 0.49 | 0.49 | 0.49 |
| transport | | | | | | |
| (GO:0099111) | | | | | | |
| negative regulation | 0.49 | 0.49 | 0.49 | 0.49 | 0.49 | 0.49 |
| of signaling | | | | | | |
| (GO:0023057) | | | | | | |
| endocardial cushion | 0.49 | 0.49 | 0.49 | 0.49 | 0.49 | 0.49 |
| formation | | | | | | |
| (GO:0003272) | | | | | | |
| negative regulation | 0.49 | 0.48 | 0.49 | 0.49 | 0.50 | 0.49 |
| of response to | | | | | | |
| stimulus | | | | | | |
| (GO:0048585) | | | | | | |
| nucleobase- | 0.49 | 0.48 | 0.49 | 0.48 | 0.49 | 0.49 |
| containing | | | | | | |
| compound | | | | | | |
| biosynthetic process | | | | | | |
| (GO:0034654) | | | | | | |
| chromosome | 0.49 | 0.48 | 0.49 | 0.49 | 0.49 | 0.48 |
| segregation | | | | | | |
| (GO:0007059) | | | | | | |
| regulation of protein | 0.49 | 0.48 | 0.49 | 0.49 | 0.49 | 0.48 |
| metabolic process | | | | | | |
| (GO:0051246) | | | | | | |
| negative regulation | 0.48 | 0.49 | 0.49 | 0.48 | 0.49 | 0.48 |
| of cell | | | | | | |
| communication | | | | | | |
| (GO:0010648) | | | | | | |
| regulation of | 0.49 | 0.48 | 0.49 | 0.48 | 0.49 | 0.49 |
| extracellular matrix | | | | | | |
| organization | | | | | | |
| (GO:1903053) | | | | | | |
| pericyte cell | 0.48 | 0.49 | 0.48 | 0.48 | 0.49 | 0.49 |
| differentiation | | | | | | |
| (GO:1904238) | | | | | | |
| regulation of protein | 0.48 | 0.48 | 0.48 | 0.48 | 0.49 | 0.49 |
| metabolic process | | | | | | |
| (GO:0051246) | | | | | | |
| regulation of cell- | 0.49 | 0.48 | 0.49 | 0.48 | 0.49 | 0.49 |
| substrate adhesion | | | | | | |
| (GO:0010810) | | | | | | |
| negative regulation | 0.48 | 0.48 | 0.48 | 0.48 | 0.49 | 0.49 |
| of signal | | | | | | |
| transduction | | | | | | |
| (GO:0009968) | | | | | | |
| regulation of | 0.49 | 0.49 | 0.49 | 0.48 | 0.49 | 0.49 |
| protein-containing | | | | | | |
| complex assembly | | | | | | |
| (GO:0043254) | | | | | | |
| negative regulation | 0.48 | 0.48 | 0.48 | 0.48 | 0.49 | 0.49 |
| of cell migration | | | | | | |
| (GO:0030336) | | | | | | |
| positive regulation | 0.48 | 0.48 | 0.48 | 0.49 | 0.49 | 0.48 |
| of nitrogen | | | | | | |
| compound metabolic | | | | | | |
| process | | | | | | |
| (GO:0051173) | | | | | | |
| positive regulation | 0.49 | 0.49 | 0.49 | 0.48 | 0.49 | 0.48 |
| of epithelial to | | | | | | |
| mesenchymal | | | | | | |
| transition | | | | | | |
| (GO:0010718) | | | | | | |
| negative regulation | 0.48 | 0.48 | 0.48 | 0.48 | 0.49 | 0.49 |
| of cell motility | | | | | | |
| (GO:2000146) | | | | | | |
| regulation of | 0.48 | 0.48 | 0.49 | 0.49 | 0.49 | 0.48 |
| metaphase plate | | | | | | |
| congression | | | | | | |
| (GO:0090235) | | | | | | |
| heart morphogenesis | 0.48 | 0.48 | 0.48 | 0.48 | 0.48 | 0.48 |
| (GO:0003007) | | | | | | |
| positive regulation | 0.48 | 0.48 | 0.48 | 0.48 | 0.49 | 0.48 |
| of molecular | | | | | | |
| function | | | | | | |
| (GO:0044093) | | | | | | |
| regulation of | 0.48 | 0.48 | 0.48 | 0.48 | 0.49 | 0.49 |
| response to external | | | | | | |
| stimulus | | | | | | |
| (GO:0032101) | | | | | | |
| positive regulation | 0.48 | 0.48 | 0.49 | 0.48 | 0.49 | 0.49 |
| of cellular process | | | | | | |
| (GO:0048522) | | | | | | |
| cytokinetic process | 0.48 | 0.48 | 0.48 | 0.49 | 0.49 | 0.48 |
| (GO:0032506) | | | | | | |
| regulation of | 0.48 | 0.48 | 0.48 | 0.48 | 0.48 | 0.48 |
| epithelial to | | | | | | |
| mesenchymal | | | | | | |
| transition | | | | | | |
| (GO:0010717) | | | | | | |
| regulation of | 0.48 | 0.48 | 0.48 | 0.48 | 0.48 | 0.48 |
| nucleobase- | | | | | | |
| containing | | | | | | |
| compound metabolic | | | | | | |
| process | | | | | | |
| (GO:0019219) | | | | | | |
| cell-matrix adhesion | 0.48 | 0.48 | 0.48 | 0.48 | 0.49 | 0.48 |
| (GO:0007160) | | | | | | |
| response to nitrogen | 0.48 | 0.48 | 0.48 | 0.48 | 0.48 | 0.48 |
| compound | | | | | | |
| (GO:1901698) | | | | | | |
| phosphorus | 0.48 | 0.48 | 0.48 | 0.48 | 0.48 | 0.48 |
| metabolic process | | | | | | |
| (GO:0006793) | | | | | | |
| positive regulation | 0.48 | 0.48 | 0.48 | 0.48 | 0.48 | 0.48 |
| of cellular process | | | | | | |
| (GO:0048522) | | | | | | |
| regulation of protein | 0.48 | 0.48 | 0.48 | 0.48 | 0.49 | 0.48 |
| modification process | | | | | | |
| (GO:0031399) | | | | | | |
| positive regulation | 0.48 | 0.48 | 0.48 | 0.48 | 0.49 | 0.48 |
| of developmental | | | | | | |
| process | | | | | | |
| (GO:0051094) | | | | | | |
| synaptic vesicle | 0.48 | 0.48 | 0.48 | 0.48 | 0.48 | 0.48 |
| recycling | | | | | | |
| (GO:0036465) | | | | | | |
| import into cell | 0.48 | 0.48 | 0.48 | 0.48 | 0.48 | 0.48 |
| (GO:0098657) | | | | | | |
| protein lipidation | 0.48 | 0.48 | 0.48 | 0.48 | 0.48 | 0.48 |
| (GO:0006497) | | | | | | |
| secretion | 0.48 | 0.48 | 0.48 | 0.48 | 0.48 | 0.48 |
| (GO:0046903) | | | | | | |
| membranous septum | 0.48 | 0.48 | 0.48 | 0.48 | 0.48 | 0.48 |
| morphogenesis | | | | | | |
| (GO:0003149) | | | | | | |
| regulation of cellular | 0.48 | 0.48 | 0.48 | 0.48 | 0.48 | 0.48 |
| component | | | | | | |
| organization | | | | | | |
| (GO:0051128) | | | | | | |
| positive regulation | 0.48 | 0.48 | 0.48 | 0.48 | 0.49 | 0.48 |
| of response to | | | | | | |
| stimulus | | | | | | |
| (GO:0048584) | | | | | | |
| cellular component | 0.48 | 0.48 | 0.48 | 0.48 | 0.48 | 0.48 |
| organization | | | | | | |
| (GO:0016043) | | | | | | |
| cellular component | 0.48 | 0.48 | 0.48 | 0.48 | 0.48 | 0.48 |
| organization or | | | | | | |
| biogenesis | | | | | | |
| (GO:0071840) | | | | | | |
| cellular response to | 0.48 | 0.48 | 0.48 | 0.48 | 0.48 | 0.48 |
| stress (GO:0033554) | | | | | | |
| regulation of mitotic | 0.48 | 0.48 | 0.48 | 0.48 | 0.48 | 0.48 |
| sister chromatid | | | | | | |
| separation | | | | | | |
| (GO:0010965) | | | | | | |
| positive regulation | 0.48 | 0.48 | 0.48 | 0.48 | 0.48 | 0.48 |
| of locomotion | | | | | | |
| (GO:0040017) | | | | | | |
| epithelial cell | 0.48 | 0.48 | 0.48 | 0.48 | 0.48 | 0.48 |
| proliferation | | | | | | |
| (GO:0050673) | | | | | | |
| positive regulation | 0.48 | 0.48 | 0.48 | 0.48 | 0.48 | 0.48 |
| of cell migration | | | | | | |
| (GO:0030335) | | | | | | |
| cellular response to | 0.48 | 0.48 | 0.48 | 0.48 | 0.48 | 0.48 |
| vascular endothelial | | | | | | |
| growth factor | | | | | | |
| stimulus | | | | | | |
| (GO:0035924) | | | | | | |
| positive regulation | 0.48 | 0.48 | 0.48 | 0.48 | 0.48 | 0.48 |
| of cell cycle | | | | | | |
| (GO:0045787) | | | | | | |
| regulation of mitotic | 0.48 | 0.48 | 0.48 | 0.48 | 0.48 | 0.48 |
| nuclear division | | | | | | |
| (GO:0007088) | | | | | | |
| branching involved | 0.48 | 0.48 | 0.48 | 0.48 | 0.48 | 0.48 |
| in ureteric bud | | | | | | |
| morphogenesis | | | | | | |
| (GO:0001658) | | | | | | |
| mitotic cytokinesis | 0.48 | 0.48 | 0.48 | 0.48 | 0.48 | 0.48 |
| (GO:0000281) | | | | | | |
| axon development | 0.48 | 0.48 | 0.48 | 0.48 | 0.48 | 0.48 |
| (GO:0061564) | | | | | | |
| mesonephros | 0.48 | 0.48 | 0.48 | 0.48 | 0.48 | 0.48 |
| development | | | | | | |
| (GO:0001823) | | | | | | |
| cell-cell adhesion | 0.48 | 0.48 | 0.48 | 0.48 | 0.48 | 0.48 |
| (GO:0098609) | | | | | | |
| regulation of | 0.48 | 0.48 | 0.48 | 0.48 | 0.48 | 0.48 |
| epithelial cell | | | | | | |
| proliferation | | | | | | |
| (GO:0050678) | | | | | | |
| animal organ | 0.48 | 0.48 | 0.48 | 0.48 | 0.48 | 0.48 |
| morphogenesis | | | | | | |
| (GO:0009887) | | | | | | |
| axonogenesis | 0.48 | 0.48 | 0.48 | 0.48 | 0.48 | 0.48 |
| (GO:0007409) | | | | | | |
| negative regulation | 0.48 | 0.48 | 0.48 | 0.48 | 0.48 | 0.48 |
| of cell development | | | | | | |
| (GO:0010721) | | | | | | |
| developmental | 0.48 | 0.48 | 0.48 | 0.48 | 0.48 | 0.48 |
| growth | | | | | | |
| (GO:0048589) | | | | | | |
| positive regulation of | 0.48 | 0.48 | 0.48 | 0.48 | 0.48 | 0.48 |
| phosphatidylinositol | | | | | | |
| 3-kinase/protein | | | | | | |
| kinase B signal | | | | | | |
| transduction | | | | | | |
| (GO:0051897) | | | | | | |
| positive regulation | 0.48 | 0.48 | 0.48 | 0.48 | 0.48 | 0.48 |
| of epithelial cell | | | | | | |
| proliferation | | | | | | |
| (GO:0050679) | | | | | | |
| regulation of | 0.48 | 0.48 | 0.48 | 0.48 | 0.48 | 0.48 |
| chromosome | | | | | | |
| separation | | | | | | |
| (GO:1905818) | | | | | | |
| regulation of | 0.48 | 0.48 | 0.48 | 0.48 | 0.48 | 0.48 |
| endothelial cell | | | | | | |
| proliferation | | | | | | |
| (GO:0001936) | | | | | | |
| mitotic sister | 0.48 | 0.48 | 0.48 | 0.48 | 0.48 | 0.48 |
| chromatid | | | | | | |
| segregation | | | | | | |
| (GO:0000070) | | | | | | |
| positive regulation | 0.48 | 0.48 | 0.48 | 0.48 | 0.48 | 0.48 |
| of phosphorus | | | | | | |
| metabolic process | | | | | | |
| (GO:0010562) | | | | | | |
| chemotaxis | 0.48 | 0.48 | 0.48 | 0.48 | 0.48 | 0.48 |
| (GO:0006935) | | | | | | |
| cytoskeleton- | 0.48 | 0.48 | 0.48 | 0.48 | 0.48 | 0.48 |
| dependent | | | | | | |
| cytokinesis | | | | | | |
| (GO:0061640) | | | | | | |
| response to growth | 0.48 | 0.48 | 0.48 | 0.48 | 0.48 | 0.48 |
| factor | | | | | | |
| (GO:0070848) | | | | | | |
| regulation of | 0.48 | 0.48 | 0.48 | 0.48 | 0.48 | 0.48 |
| transmembrane | | | | | | |
| receptor protein | | | | | | |
| serine/threonine | | | | | | |
| kinase signaling | | | | | | |
| pathway | | | | | | |
| (GO:0090092) | | | | | | |
| mesonephric tubule | 0.48 | 0.48 | 0.48 | 0.48 | 0.48 | 0.48 |
| morphogenesis | | | | | | |
| (GO:0072171) | | | | | | |
| regulation of Wnt | 0.48 | 0.48 | 0.48 | 0.48 | 0.48 | 0.48 |
| signaling pathway | | | | | | |
| (GO:0030111) | | | | | | |
| negative regulation | 0.48 | 0.48 | 0.48 | 0.48 | 0.48 | 0.48 |
| of cellular response | | | | | | |
| to growth factor | | | | | | |
| stimulus | | | | | | |
| (GO:0090288) | | | | | | |
| epithelial tube | 0.48 | 0.48 | 0.48 | 0.48 | 0.48 | 0.48 |
| morphogenesis | | | | | | |
| (GO:0060562) | | | | | | |
| positive regulation | 0.48 | 0.48 | 0.48 | 0.48 | 0.48 | 0.48 |
| of phosphorylation | | | | | | |
| (GO:0042327) | | | | | | |
| positive regulation | 0.48 | 0.48 | 0.48 | 0.48 | 0.48 | 0.48 |
| of intracellular | | | | | | |
| signal transduction | | | | | | |
| (GO:1902533) | | | | | | |
| tube morphogenesis | 0.48 | 0.48 | 0.48 | 0.48 | 0.48 | 0.48 |
| (GO:0035239) | | | | | | |
| cell cycle phase | 0.48 | 0.48 | 0.48 | 0.48 | 0.48 | 0.48 |
| transition | | | | | | |
| (GO:0044770) | | | | | | |
| positive regulation | 0.48 | 0.48 | 0.48 | 0.48 | 0.48 | 0.48 |
| of chromosome | | | | | | |
| separation | | | | | | |
| (GO:1905820) | | | | | | |
| kidney development | 0.48 | 0.48 | 0.48 | 0.48 | 0.48 | 0.48 |
| (GO:0001822) | | | | | | |
| central nervous | 0.48 | 0.48 | 0.48 | 0.48 | 0.48 | 0.48 |
| system development | | | | | | |
| (GO:0007417) | | | | | | |
| circulatory system | 0.48 | 0.48 | 0.48 | 0.48 | 0.48 | 0.48 |
| development | | | | | | |
| (GO:0072359) | | | | | | |
| growth | 0.48 | 0.48 | 0.48 | 0.48 | 0.48 | 0.48 |
| (GO:0040007) | | | | | | |
| chromosome | 0.48 | 0.48 | 0.48 | 0.48 | 0.48 | 0.48 |
| localization | | | | | | |
| (GO:0050000) | | | | | | |
| protein | 0.48 | 0.48 | 0.48 | 0.48 | 0.48 | 0.48 |
| phosphorylation | | | | | | |
| (GO:0006468) | | | | | | |
| ossification | 0.48 | 0.48 | 0.48 | 0.48 | 0.48 | 0.48 |
| (GO:0001503) | | | | | | |
| anatomical structure | 0.48 | 0.48 | 0.48 | 0.48 | 0.48 | 0.48 |
| morphogenesis | | | | | | |
| (GO:0009653) | | | | | | |
| regulation of protein | 0.48 | 0.48 | 0.48 | 0.48 | 0.48 | 0.48 |
| phosphorylation | | | | | | |
| (GO:0001932) | | | | | | |
| positive regulation | 0.48 | 0.48 | 0.48 | 0.48 | 0.48 | 0.48 |
| of endothelial cell | | | | | | |
| proliferation | | | | | | |
| (GO:0001938) | | | | | | |
| establishment of | 0.48 | 0.48 | 0.48 | 0.48 | 0.48 | 0.48 |
| chromosome | | | | | | |
| localization | | | | | | |
| (GO:0051303) | | | | | | |
| positive regulation | 0.48 | 0.48 | 0.48 | 0.48 | 0.48 | 0.48 |
| of protein metabolic | | | | | | |
| process | | | | | | |
| (GO:0051247) | | | | | | |
| angiogenesis | 0.48 | 0.48 | 0.48 | 0.48 | 0.48 | 0.48 |
| (GO:0001525) | | | | | | |
| osteoblast | 0.48 | 0.48 | 0.48 | 0.48 | 0.48 | 0.48 |
| differentiation | | | | | | |
| (GO:0001649) | | | | | | |
| neuron projection | 0.48 | 0.48 | 0.48 | 0.48 | 0.48 | 0.48 |
| development | | | | | | |
| (GO:0031175) | | | | | | |
| cell chemotaxis | 0.48 | 0.48 | 0.48 | 0.48 | 0.48 | 0.48 |
| (GO:0060326) | | | | | | |
| wound healing | 0.48 | 0.48 | 0.48 | 0.48 | 0.48 | 0.48 |
| (GO:0042060) | | | | | | |
| cell morphogenesis | 0.48 | 0.48 | 0.48 | 0.48 | 0.48 | 0.48 |
| (GO:0000902) | | | | | | |
| phosphorylation | 0.48 | 0.48 | 0.48 | 0.48 | 0.48 | 0.48 |
| (GO:0016310) | | | | | | |
| regulation of | 0.48 | 0.48 | 0.48 | 0.48 | 0.48 | 0.48 |
| chromosome | | | | | | |
| segregation | | | | | | |
| (GO:0051983) | | | | | | |
| bone development | 0.48 | 0.48 | 0.48 | 0.48 | 0.48 | 0.48 |
| (GO:0060348) | | | | | | |
| positive regulation | 0.48 | 0.48 | 0.48 | 0.48 | 0.48 | 0.48 |
| of chemotaxis | | | | | | |
| (GO:0050921) | | | | | | |
| microtubule | 0.48 | 0.48 | 0.48 | 0.48 | 0.48 | 0.48 |
| cytoskeleton | | | | | | |
| organization | | | | | | |
| involved in mitosis | | | | | | |
| (GO:1902850) | | | | | | |
| regulation of protein | 0.48 | 0.48 | 0.48 | 0.48 | 0.48 | 0.48 |
| modification process | | | | | | |
| (GO:0031399) | | | | | | |
| regulation of ERK1 | 0.48 | 0.48 | 0.48 | 0.48 | 0.48 | 0.48 |
| and ERK2 cascade | | | | | | |
| (GO:0070372) | | | | | | |
| response to | 0.48 | 0.48 | 0.48 | 0.48 | 0.48 | 0.48 |
| wounding | | | | | | |
| (GO:0009611) | | | | | | |
| apoptotic process | 0.48 | 0.48 | 0.48 | 0.48 | 0.48 | 0.48 |
| (GO:0006915) | | | | | | |
| positive regulation | 0.48 | 0.48 | 0.48 | 0.48 | 0.48 | 0.48 |
| of cell-substrate | | | | | | |
| adhesion | | | | | | |
| (GO:0010811) | | | | | | |
| positive regulation | 0.48 | 0.48 | 0.48 | 0.48 | 0.48 | 0.48 |
| of cell adhesion | | | | | | |
| (GO:0045785) | | | | | | |
| neuron development | 0.48 | 0.48 | 0.48 | 0.48 | 0.48 | 0.48 |
| (GO:0048666) | | | | | | |
| cell death | 0.48 | 0.48 | 0.48 | 0.48 | 0.48 | 0.48 |
| (GO:0008219) | | | | | | |
| programned cell | 0.48 | 0.48 | 0.48 | 0.48 | 0.48 | 0.48 |
| death (GO:0012501) | | | | | | |
| regulation of | 0.48 | 0.48 | 0.48 | 0.48 | 0.48 | 0.48 |
| multicellular | | | | | | |
| organismal | | | | | | |
| development | | | | | | |
| (GO:2000026) | | | | | | |
| regulation of | 0.48 | 0.48 | 0.48 | 0.48 | 0.48 | 0.48 |
| endothelial cell | | | | | | |
| apoptotic process | | | | | | |
| (GO:2000351) | | | | | | |
| nephron epithelium | 0.48 | 0.48 | 0.48 | 0.48 | 0.48 | 0.48 |
| development | | | | | | |
| (GO:0072009) | | | | | | |
| renal tubule | 0.48 | 0.48 | 0.48 | 0.48 | 0.48 | 0.48 |
| morphogenesis | | | | | | |
| (GO:0061333) | | | | | | |
| regulation of | 0.48 | 0.48 | 0.48 | 0.48 | 0.48 | 0.48 |
| programmed cell | | | | | | |
| death (GO:0043067) | | | | | | |
| protein transport | 0.48 | 0.48 | 0.48 | 0.48 | 0.48 | 0.48 |
| (GO:0015031) | | | | | | |
| spindle organization | 0.48 | 0.48 | 0.48 | 0.48 | 0.48 | 0.48 |
| (GO:0007051) | | | | | | |
| transmembrane | 0.48 | 0.48 | 0.48 | 0.48 | 0.48 | 0.48 |
| receptor protein | | | | | | |
| tyrosine kinase | | | | | | |
| signaling pathway | | | | | | |
| (GO:0007169) | | | | | | |
| regulation of | 0.48 | 0.48 | 0.48 | 0.48 | 0.48 | 0.48 |
| phosphorylation | | | | | | |
| (GO:0042325) | | | | | | |
| kidney | 0.48 | 0.48 | 0.48 | 0.48 | 0.48 | 0.48 |
| morphogenesis | | | | | | |
| (GO:0060993) | | | | | | |
| positive regulation | 0.48 | 0.48 | 0.48 | 0.48 | 0.48 | 0.48 |
| of mitotic sister | | | | | | |
| chromatid separation | | | | | | |
| (GO:1901970) | | | | | | |
| positive regulation | 0.48 | 0.48 | 0.48 | 0.48 | 0.48 | 0.48 |
| of biological process | | | | | | |
| (GO:0048518) | | | | | | |
| secretion by cell | 0.49 | 0.48 | 0.48 | 0.48 | 0.48 | 0.48 |
| (GO:0032940) | | | | | | |
| negative regulation | 0.48 | 0.48 | 0.48 | 0.48 | 0.48 | 0.48 |
| of apoptotic process | | | | | | |
| (GO:0043066) | | | | | | |
| phosphorus | 0.48 | 0.48 | 0.48 | 0.48 | 0.48 | 0.48 |
| metabolic process | | | | | | |
| (GO:0006793) | | | | | | |
| regulation of | 0.48 | 0.48 | 0.48 | 0.48 | 0.48 | 0.48 |
| hydrolase activity | | | | | | |
| (GO:0051336) | | | | | | |
| regulation of cell | 0.48 | 0.48 | 0.48 | 0.48 | 0.48 | 0.48 |
| projection | | | | | | |
| organization | | | | | | |
| (GO:0031344) | | | | | | |
| phosphate- | 0.48 | 0.48 | 0.48 | 0.48 | 0.48 | 0.48 |
| containing | | | | | | |
| compound metabolic | | | | | | |
| process | | | | | | |
| (GO:0006796) | | | | | | |
| regulation of | 0.48 | 0.48 | 0.48 | 0.48 | 0.48 | 0.48 |
| intracellular signal | | | | | | |
| transduction | | | | | | |
| (GO:1902531) | | | | | | |
| positive regulation | 0.48 | 0.48 | 0.48 | 0.48 | 0.48 | 0.48 |
| of catalytic activity | | | | | | |
| (GO:0043085) | | | | | | |
| regulation of | 0.48 | 0.48 | 0.48 | 0.48 | 0.48 | 0.48 |
| response to stress | | | | | | |
| (GO:0080134) | | | | | | |
| nervous system | 0.48 | 0.48 | 0.48 | 0.48 | 0.48 | 0.48 |
| development | | | | | | |
| (GO:0007399) | | | | | | |
| cell junction | 0.48 | 0.48 | 0.48 | 0.48 | 0.48 | 0.48 |
| assembly | | | | | | |
| (GO:0034329) | | | | | | |
| regulation of cell | 0.48 | 0.48 | 0.48 | 0.48 | 0.48 | 0.48 |
| adhesion | | | | | | |
| (GO:0030155) | | | | | | |
| positive regulation | 0.48 | 0.48 | 0.48 | 0.48 | 0.48 | 0.48 |
| of molecular | | | | | | |
| function | | | | | | |
| (GO:0044093) | | | | | | |
| digestive tract | 0.48 | 0.48 | 0.48 | 0.48 | 0.48 | 0.48 |
| morphogenesis | | | | | | |
| (GO:0048546) | | | | | | |
| positive regulation | 0.48 | 0.48 | 0.48 | 0.48 | 0.48 | 0.48 |
| of transmembrane | | | | | | |
| receptor protein | | | | | | |
| serine/threonine | | | | | | |
| kinase signaling | | | | | | |
| pathway | | | | | | |
| (GO:0090100) | | | | | | |
| positive regulation | 0.48 | 0.48 | 0.48 | 0.48 | 0.48 | 0.48 |
| of hydrolase activity | | | | | | |
| (GO:0051345) | | | | | | |
| positive regulation | 0.48 | 0.48 | 0.48 | 0.48 | 0.48 | 0.48 |
| of mitotic | | | | | | |
| cytokinesis | | | | | | |
| (GO:1903490) | | | | | | |
| regulation of body | 0.48 | 0.48 | 0.48 | 0.48 | 0.48 | 0.48 |
| fluid levels | | | | | | |
| (GO:0050878) | | | | | | |
| response to abiotic | 0.48 | 0.48 | 0.48 | 0.48 | 0.48 | 0.48 |
| stimulus | | | | | | |
| (GO:0009628) | | | | | | |
| positive regulation | 0.48 | 0.48 | 0.48 | 0.48 | 0.48 | 0.48 |
| of chromosome | | | | | | |
| segregation | | | | | | |
| (GO:0051984) | | | | | | |
| cell division | 0.48 | 0.48 | 0.48 | 0.48 | 0.48 | 0.48 |
| (GO:0051301) | | | | | | |
| response to | 0.48 | 0.48 | 0.48 | 0.48 | 0.48 | 0.48 |
| endogenous stimulus | | | | | | |
| (GO:0009719) | | | | | | |
| cell cycle checkpoint | 0.48 | 0.48 | 0.48 | 0.48 | 0.48 | 0.48 |
| signaling | | | | | | |
| (GO:0000075) | | | | | | |
| negative regulation | 0.48 | 0.48 | 0.48 | 0.48 | 0.48 | 0.48 |
| of cellular | | | | | | |
| component | | | | | | |
| organization | | | | | | |
| (GO:0051129) | | | | | | |
| response to | 0.48 | 0.48 | 0.48 | 0.48 | 0.48 | 0.48 |
| inorganic substance | | | | | | |
| (GO:0010035) | | | | | | |
| vasculogenesis | 0.48 | 0.48 | 0.48 | 0.48 | 0.48 | 0.48 |
| (GO:0001570) | | | | | | |
| regulation of cell- | 0.48 | 0.48 | 0.48 | 0.48 | 0.48 | 0.48 |
| matrix adhesion | | | | | | |
| (GO:0001952) | | | | | | |
| inflammatory | 0.48 | 0.48 | 0.48 | 0.48 | 0.48 | 0.48 |
| response | | | | | | |
| (GO:0006954) | | | | | | |
| bone mineralization | 0.48 | 0.48 | 0.48 | 0.48 | 0.48 | 0.48 |
| (GO:0030282) | | | | | | |
| blood vessel | 0.48 | 0.48 | 0.48 | 0.48 | 0.48 | 0.48 |
| endothelial cell | | | | | | |
| proliferation | | | | | | |
| involved in | | | | | | |
| sprouting | | | | | | |
| angiogenesis | | | | | | |
| (GO:0002043) | | | | | | |
| positive regulation | 0.48 | 0.48 | 0.48 | 0.48 | 0.48 | 0.48 |
| of cellular metabolic | | | | | | |
| process | | | | | | |
| (GO:0031325) | | | | | | |
| positive regulation | 0.48 | 0.48 | 0.48 | 0.48 | 0.48 | 0.48 |
| of neuroepithelial | | | | | | |
| cell differentiation | | | | | | |
| (GO:1902913) | | | | | | |
| regulation of | 0.48 | 0.48 | 0.48 | 0.48 | 0.48 | 0.48 |
| developmental | | | | | | |
| process | | | | | | |
| (GO:0050793) | | | | | | |
| cell cycle | 0.48 | 0.48 | 0.48 | 0.48 | 0.48 | 0.48 |
| (GO:0007049) | | | | | | |
| cell cycle process | 0.48 | 0.48 | 0.48 | 0.48 | 0.48 | 0.48 |
| (GO:0022402) | | | | | | |
| positive regulation | 0.48 | 0.48 | 0.48 | 0.48 | 0.48 | 0.48 |
| of metabolic process | | | | | | |
| (GO:0009893) | | | | | | |
| mitotic cell cycle | 0.48 | 0.48 | 0.48 | 0.48 | 0.48 | 0.48 |
| process | | | | | | |
| (GO:1903047) | | | | | | |
| regulation of | 0.48 | 0.48 | 0.48 | 0.48 | 0.48 | 0.48 |
| nitrogen compound | | | | | | |
| metabolic process | | | | | | |
| (GO:0051171) | | | | | | |
| tissue development | 0.48 | 0.48 | 0.48 | 0.48 | 0.48 | 0.48 |
| (GO:0009888) | | | | | | |
| aromatic compound | 0.48 | 0.48 | 0.48 | 0.48 | 0.48 | 0.48 |
| biosynthetic process | | | | | | |
| (GO:0019438) | | | | | | |
| metaphase | 0.48 | 0.48 | 0.48 | 0.48 | 0.48 | 0.48 |
| chromosome | | | | | | |
| alignment | | | | | | |
| (GO:0051310) | | | | | | |
| mitotic cell cycle | 0.48 | 0.48 | 0.48 | 0.48 | 0.48 | 0.48 |
| (GO:0000278) | | | | | | |
| nucleobase- | 0.48 | 0.48 | 0.48 | 0.48 | 0.48 | 0.48 |
| containing | | | | | | |
| compound | | | | | | |
| biosynthetic process | | | | | | |
| (GO:0034654) | | | | | | |
| regulation of RNA | 0.48 | 0.48 | 0.48 | 0.48 | 0.48 | 0.48 |
| metabolic process | | | | | | |
| (GO:0051252) | | | | | | |
| connective tissue | 0.48 | 0.48 | 0.48 | 0.48 | 0.48 | 0.48 |
| development | | | | | | |
| (GO:0061448) | | | | | | |
| regulation of | 0.48 | 0.48 | 0.48 | 0.48 | 0.48 | 0.48 |
| cytoskeleton | | | | | | |
| organization | | | | | | |
| (GO:0051493) | | | | | | |
| intracellular | 0.48 | 0.48 | 0.48 | 0.48 | 0.48 | 0.48 |
| transport | | | | | | |
| (GO:0046907) | | | | | | |
| aromatic compound | 0.48 | 0.48 | 0.48 | 0.48 | 0.48 | 0.48 |
| biosynthetic process | | | | | | |
| (GO:0019438) | | | | | | |
| chromosome | 0.48 | 0.48 | 0.48 | 0.48 | 0.48 | 0.48 |
| segregation | | | | | | |
| (GO:0007059) | | | | | | |
| regulation of | 0.48 | 0.48 | 0.48 | 0.48 | 0.48 | 0.48 |
| molecular function | | | | | | |
| (GO:0065009) | | | | | | |
| negative regulation | 0.48 | 0.48 | 0.48 | 0.48 | 0.48 | 0.48 |
| of nitrogen | | | | | | |
| compound metabolic | | | | | | |
| process | | | | | | |
| (GO:0051172) | | | | | | |
| regulation of signal | 0.48 | 0.48 | 0.48 | 0.48 | 0.48 | 0.48 |
| transduction | | | | | | |
| (GO:0009966) | | | | | | |
| negative regulation | 0.48 | 0.48 | 0.48 | 0.48 | 0.48 | 0.48 |
| of cellular | | | | | | |
| component | | | | | | |
| organization | | | | | | |
| (GO:0051129) | | | | | | |
| positive regulation | 0.48 | 0.48 | 0.48 | 0.48 | 0.48 | 0.48 |
| of cell cycle process | | | | | | |
| (GO:0090068) | | | | | | |
| cell surface receptor | 0.48 | 0.48 | 0.48 | 0.48 | 0.48 | 0.48 |
| signaling pathway | | | | | | |
| (GO:0007166) | | | | | | |
| nitrogen compound | 0.48 | 0.48 | 0.48 | 0.48 | 0.48 | 0.48 |
| transport | | | | | | |
| (GO:0071705) | | | | | | |
| response to peptide | 0.48 | 0.48 | 0.48 | 0.48 | 0.48 | 0.48 |
| (GO:1901652) | | | | | | |
| regulation of | 0.48 | 0.48 | 0.48 | 0.48 | 0.48 | 0.48 |
| intracellular signal | | | | | | |
| transduction | | | | | | |
| (GO:1902531) | | | | | | |
| organic substance | 0.48 | 0.48 | 0.48 | 0.48 | 0.48 | 0.48 |
| transport | | | | | | |
| (GO:0071702) | | | | | | |
| cell adhesion | 0.48 | 0.48 | 0.48 | 0.48 | 0.48 | 0.48 |
| (GO:0007155) | | | | | | |
| negative regulation | 0.48 | 0.48 | 0.48 | 0.48 | 0.48 | 0.48 |
| of organelle | | | | | | |
| organization | | | | | | |
| (GO:0010639) | | | | | | |
| establishment of | 0.48 | 0.48 | 0.48 | 0.48 | 0.48 | 0.48 |
| localization in cell | | | | | | |
| (GO:0051649) | | | | | | |
| cellular response to | 0.48 | 0.48 | 0.48 | 0.48 | 0.48 | 0.48 |
| stimulus | | | | | | |
| (GO:0051716) | | | | | | |
| mitotic nuclear | 0.48 | 0.48 | 0.48 | 0.48 | 0.48 | 0.48 |
| division | | | | | | |
| (GO:0140014) | | | | | | |
| regulation of | 0.48 | 0.48 | 0.48 | 0.48 | 0.48 | 0.48 |
| response to stimulus | | | | | | |
| (GO:0048583) | | | | | | |
| positive regulation | 0.48 | 0.48 | 0.48 | 0.48 | 0.48 | 0.48 |
| of cell | | | | | | |
| communication | | | | | | |
| (GO:0010647) | | | | | | |
| chromosome | 0.48 | 0.48 | 0.48 | 0.48 | 0.48 | 0.48 |
| separation | | | | | | |
| (GO:0051304) | | | | | | |
| regulation of | 0.48 | 0.48 | 0.48 | 0.48 | 0.48 | 0.48 |
| phosphorylation | | | | | | |
| (GO:0042325) | | | | | | |
| regulation of cell | 0.48 | 0.48 | 0.48 | 0.48 | 0.48 | 0.48 |
| growth | | | | | | |
| (GO:0001558) | | | | | | |
| positive regulation | 0.48 | 0.48 | 0.48 | 0.48 | 0.48 | 0.48 |
| of cell | | | | | | |
| communication | | | | | | |
| (GO:0010647) | | | | | | |
| regulation of | 0.48 | 0.48 | 0.48 | 0.48 | 0.48 | 0.48 |
| multicellular | | | | | | |
| organismal process | | | | | | |
| (GO:0051239) | | | | | | |
| blood vessel | 0.48 | 0.48 | 0.48 | 0.48 | 0.48 | 0.48 |
| development | | | | | | |
| (GO:0001568) | | | | | | |
| sister chromatid | 0.48 | 0.48 | 0.48 | 0.48 | 0.48 | 0.48 |
| segregation | | | | | | |
| (GO:0000819) | | | | | | |
| regulation of | 0.48 | 0.48 | 0.48 | 0.48 | 0.48 | 0.48 |
| apoptotic process | | | | | | |
| (GO:0042981) | | | | | | |
| microtubule | 0.48 | 0.48 | 0.48 | 0.48 | 0.48 | 0.48 |
| cytoskeleton | | | | | | |
| organization | | | | | | |
| (GO:0000226) | | | | | | |
| organelle fission | 0.48 | 0.48 | 0.48 | 0.48 | 0.48 | 0.48 |
| (GO:0048285) | | | | | | |
| hemostasis | 0.48 | 0.48 | 0.48 | 0.48 | 0.48 | 0.48 |
| (GO:0007599) | | | | | | |
| regulation of protein | 0.48 | 0.48 | 0.48 | 0.48 | 0.48 | 0.48 |
| phosphorylation | | | | | | |
| (GO:0001932) | | | | | | |
| gastrulation | 0.48 | 0.48 | 0.48 | 0.48 | 0.48 | 0.48 |
| (GO:0007369) | | | | | | |
| regulation of cell | 0.48 | 0.48 | 0.48 | 0.48 | 0.48 | 0.48 |
| communication | | | | | | |
| (GO:0010646) | | | | | | |
| nuclear chromosome | 0.48 | 0.48 | 0.48 | 0.48 | 0.48 | 0.48 |
| segregation | | | | | | |
| (GO:0098813) | | | | | | |
| nuclear division | 0.48 | 0.48 | 0.48 | 0.48 | 0.48 | 0.48 |
| (GO:0000280) | | | | | | |
| peptidyl-amino acid | 0.48 | 0.48 | 0.48 | 0.48 | 0.48 | 0.48 |
| modification | | | | | | |
| (GO:0018193) | | | | | | |
| establishment of | 0.48 | 0.48 | 0.48 | 0.48 | 0.48 | 0.48 |
| protein localization | | | | | | |
| (GO:0045184) | | | | | | |
| regulation of | 0.48 | 0.48 | 0.48 | 0.48 | 0.48 | 0.48 |
| anatomical structure | | | | | | |
| size (GO:0090066) | | | | | | |
| positive regulation | 0.48 | 0.48 | 0.48 | 0.48 | 0.48 | 0.48 |
| of nucleobase- | | | | | | |
| containing | | | | | | |
| compound metabolic | | | | | | |
| process | | | | | | |
| (GO:0045935) | | | | | | |
| regulation of nuclear | 0.48 | 0.48 | 0.48 | 0.48 | 0.48 | 0.48 |
| division | | | | | | |
| (GO:0051783) | | | | | | |
| regulation of | 0.48 | 0.48 | 0.48 | 0.48 | 0.48 | 0.48 |
| angiogenesis | | | | | | |
| (GO:0045765) | | | | | | |
| positive regulation | 0.48 | 0.48 | 0.48 | 0.48 | 0.48 | 0.48 |
| of catalytic activity | | | | | | |
| (GO:0043085) | | | | | | |
| animal organ | 0.47 | 0.48 | 0.47 | 0.47 | 0.47 | 0.47 |
| development | | | | | | |
| (GO:0048513) | | | | | | |
| regulation of protein | 0.47 | 0.47 | 0.47 | 0.47 | 0.47 | 0.47 |
| localization | | | | | | |
| (GO:0032880) | | | | | | |
| positive regulation | 0.47 | 0.47 | 0.47 | 0.47 | 0.47 | 0.47 |
| of protein transport | | | | | | |
| (GO:0051222) | | | | | | |
| regulation of | 0.47 | 0.47 | 0.47 | 0.47 | 0.47 | 0.47 |
| secretion | | | | | | |
| (GO:0051046) | | | | | | |
| regulation of | 0.47 | 0.47 | 0.47 | 0.47 | 0.47 | 0.47 |
| establishment of | | | | | | |
| protein localization | | | | | | |
| (GO:0070201) | | | | | | |
| regulation of cellular | 0.47 | 0.47 | 0.47 | 0.47 | 0.47 | 0.47 |
| localization | | | | | | |
| (GO:0060341) | | | | | | |
| cellular | | | | | | |
| macromolecule | 0.47 | 0.47 | 0.47 | 0.47 | 0.47 | 0.47 |
| localization | | | | | | |
| (GO:0070727) | | | | | | |
| roof of mouth | 0.47 | 0.47 | 0.47 | 0.47 | 0.47 | 0.47 |
| development | | | | | | |
| (GO:0060021) | | | | | | |
| regulation of | 0.47 | 0.47 | 0.47 | 0.47 | 0.47 | 0.47 |
| secretion by cell | | | | | | |
| (GO:1903530) | | | | | | |
| TOR signaling | 0.47 | 0.47 | 0.47 | 0.47 | 0.47 | 0.47 |
| (GO:0031929) | | | | | | |
| regulation of | 0.47 | 0.47 | 0.47 | 0.47 | 0.47 | 0.47 |
| hemostasis | | | | | | |
| (GO:1900046) | | | | | | |
| metanephros | 0.47 | 0.47 | 0.47 | 0.47 | 0.47 | 0.47 |
| development | | | | | | |
| (GO:0001656) | | | | | | |
| macromolecule | 0.47 | 0.47 | 0.47 | 0.47 | 0.47 | 0.47 |
| localization | | | | | | |
| (GO:0033036) | | | | | | |
| tendon development | 0.47 | 0.47 | 0.47 | 0.47 | 0.47 | 0.47 |
| (GO:0035989) | | | | | | |
| renal system | 0.47 | 0.47 | 0.47 | 0.47 | 0.47 | 0.47 |
| vasculature | | | | | | |
| morphogenesis | | | | | | |
| (GO:0061438) | | | | | | |
| endoderm formation | 0.47 | 0.47 | 0.47 | 0.47 | 0.47 | 0.47 |
| (GO:0001706) | | | | | | |
| gland | 0.47 | 0.47 | 0.47 | 0.47 | 0.47 | 0.47 |
| morphogenesis | | | | | | |
| (GO:0022612) | | | | | | |
| negative regulation | 0.47 | 0.47 | 0.47 | 0.47 | 0.47 | 0.47 |
| of smooth muscle | | | | | | |
| cell migration | | | | | | |
| (GO:0014912) | | | | | | |
| regulation of blood | 0.47 | 0.47 | 0.47 | 0.47 | 0.47 | 0.47 |
| coagulation | | | | | | |
| (GO:0030193) | | | | | | |
| regulation of smooth | 0.47 | 0.47 | 0.47 | 0.47 | 0.47 | 0.47 |
| muscle cell | | | | | | |
| migration | | | | | | |
| (GO:0014910) | | | | | | |
| metanepbric | 0.47 | 0.47 | 0.47 | 0.47 | 0.47 | 0.47 |
| nephron | | | | | | |
| development | | | | | | |
| (GO:0072210) | | | | | | |
| artery development | 0.47 | 0.47 | 0.47 | 0.47 | 0.47 | 0.47 |
| (GO:0060840) | | | | | | |
| regulation of | 0.47 | 0.47 | 0.47 | 0.47 | 0.47 | 0.47 |
| phosphatidylinositol | | | | | | |
| 3-kinase/protein | | | | | | |
| kinase B signal | | | | | | |
| transduction | | | | | | |
| (GO:0051896) | | | | | | |
| negative regulation | 0.47 | 0.47 | 0.47 | 0.47 | 0.47 | 0.47 |
| of cartilage | | | | | | |
| development | | | | | | |
| (GO:0061037) | | | | | | |
| regulation of wound | 0.47 | 0.47 | 0.47 | 0.47 | 0.47 | 0.47 |
| healing | | | | | | |
| (GO:0061041) | | | | | | |
| bone morphogenesis | 0.47 | 0.47 | 0.47 | 0.47 | 0.47 | 0.47 |
| (GO:0060349) | | | | | | |
| organ growth | 0.47 | 0.47 | 0.47 | 0.47 | 0.47 | 0.47 |
| GO:0035265) | | | | | | |
| blood coagulation | 0.47 | 0.47 | 0.47 | 0.47 | 0.47 | 0.47 |
| (GO:0007596) | | | | | | |
| positive chemotaxis | 0.47 | 0.47 | 0.47 | 0.47 | 0.47 | 0.47 |
| (GO:0050918) | | | | | | |
| regulation of stem | 0.47 | 0.47 | 0.47 | 0.47 | 0.47 | 0.47 |
| cell proliferation | | | | | | |
| (GO:0072091) | | | | | | |
| epithelial to | 0.47 | 0.47 | 0.47 | 0.47 | 0.47 | 0.47 |
| mesenchymal | | | | | | |
| transition | | | | | | |
| (GO:0001837) | | | | | | |
| cardiac ventricle | 0.47 | 0.47 | 0.47 | 0.47 | 0.47 | 0.47 |
| development | | | | | | |
| (GO:0003231) | | | | | | |
| ureteric bud | 0.47 | 0.47 | 0.47 | 0.47 | 0.47 | 0.47 |
| morphogenesis | | | | | | |
| (GO:0060675) | | | | | | |
| regulation of | 0.47 | 0.47 | 0.47 | 0.47 | 0.47 | 0.47 |
| extracellular matrix | | | | | | |
| disassembly | | | | | | |
| (GO:0010715) | | | | | | |
| phosphatidylinositol | 0.47 | 0.47 | 0.47 | 0.47 | 0.47 | 0.47 |
| 3-kinase/protein | | | | | | |
| kinase B signal | | | | | | |
| transduction | | | | | | |
| (GO:0043491) | | | | | | |
| muscle cell | 0.47 | 0.47 | 0.47 | 0.47 | 0.47 | 0.47 |
| development | | | | | | |
| (GO:0055001) | | | | | | |
| morphogenesis of a | 0.47 | 0.47 | 0.47 | 0.47 | 0.47 | 0.47 |
| branching structure | | | | | | |
| (GO:0001763) | | | | | | |
| stem cell | 0.47 | 0.47 | 0.47 | 0.47 | 0.47 | 0.47 |
| proliferation | | | | | | |
| (GO:0072089) | | | | | | |
| positive regulation | 0.47 | 0.47 | 0.47 | 0.47 | 0.47 | 0.47 |
| of protein | | | | | | |
| phosphorylation | | | | | | |
| (GO:0001934) | | | | | | |
| neuronal stem cell | 0.47 | 0.47 | 0.47 | 0.47 | 0.47 | 0.47 |
| population | | | | | | |
| maintenance | | | | | | |
| (GO:0097150) | | | | | | |
| morphogenesis of a | 0.47 | 0.47 | 0.47 | 0.47 | 0.47 | 0.47 |
| branching | | | | | | |
| epithelium | | | | | | |
| (GO:0061138) | | | | | | |
| branching | 0.47 | 0.47 | 0.47 | 0.47 | 0.47 | 0.47 |
| morphogenesis of an | | | | | | |
| epithelial tube | | | | | | |
| (GO:0048754) | | | | | | |
| positive regulation | 0.47 | 0.47 | 0.47 | 0.47 | 0.47 | 0.47 |
| of osteoblast | | | | | | |
| differentiation | | | | | | |
| (GO:0045669) | | | | | | |
| muscle organ | 0.47 | 0.47 | 0.47 | 0.47 | 0.47 | 0.47 |
| development | | | | | | |
| (GO:0007517) | | | | | | |
| positive regulation | 0.47 | 0.47 | 0.47 | 0.47 | 0.47 | 0.47 |
| of protein | | | | | | |
| modification process | | | | | | |
| (GO:0031401) | | | | | | |
| forebrain | 0.47 | 0.47 | 0.47 | 0.47 | 0.47 | 0.47 |
| development | | | | | | |
| (GO:0030900) | | | | | | |
| muscle tissue | 0.47 | 0.47 | 0.47 | 0.47 | 0.47 | 0.47 |
| development | | | | | | |
| (GO:0060537) | | | | | | |
| brain development | 0.47 | 0.47 | 0.47 | 0.47 | 0.47 | 0.47 |
| (GO:0007420) | | | | | | |
| head development | 0.47 | 0.47 | 0.47 | 0.47 | 0.47 | 0.47 |
| (GO:0060322) | | | | | | |
| cell growth | 0.47 | 0.47 | 0.47 | 0.47 | 0.47 | 0.47 |
| (GO:0016049) | | | | | | |
| positive regulation | 0.47 | 0.47 | 0.47 | 0.47 | 0.47 | 0.47 |
| of protein metabolic | | | | | | |
| process | | | | | | |
| (GO:0051247) | | | | | | |
| mitotic cell cycle | 0.47 | 0.47 | 0.47 | 0.47 | 0.47 | 0.47 |
| process | | | | | | |
| GO:1903047) | | | | | | |
| microtubule-based | 0.47 | 0.47 | 0.47 | 0.47 | 0.47 | 0.47 |
| process | | | | | | |
| (GO:0007017) | | | | | | |
| cell surface receptor | 0.47 | 0.47 | 0.47 | 0.47 | 0.47 | 0.47 |
| signaling pathway | | | | | | |
| (GO:0007166) | | | | | | |
| negative regulation | 0.47 | 0.47 | 0.47 | 0.47 | 0.47 | 0.47 |
| of cellular process | | | | | | |
| (GO:0048523) | | | | | | |
| extracellular matrix | 0.47 | 0.47 | 0.47 | 0.47 | 0.47 | 0.47 |
| organization | | | | | | |
| GO:0030198) | | | | | | |
| organic cyclic | 0.47 | 0.47 | 0.47 | 0.47 | 0.47 | 0.47 |
| compound | | | | | | |
| biosynthetic process | | | | | | |
| (GO:1901362) | | | | | | |
| cellular nitrogen | 0.47 | 0.47 | 0.47 | 0.47 | 0.47 | 0.47 |
| compound | | | | | | |
| biosynthetic process | | | | | | |
| (GO:0044271) | | | | | | |
| autophagosome | 0.47 | 0.47 | 0.47 | 0.47 | 0.47 | 0.47 |
| organization | | | | | | |
| (GO:1905037) | | | | | | |
| positive regulation | 0.47 | 0.47 | 0.47 | 0.47 | 0.47 | 0.47 |
| of protein | | | | | | |
| modification process | | | | | | |
| (GO:0031401) | | | | | | |
| embryo | 0.47 | 0.47 | 0.47 | 0.47 | 0.47 | 0.47 |
| development | | | | | | |
| (GO:0009790) | | | | | | |
| negative regulation | 0.47 | 0.47 | 0.47 | 0.47 | 0.47 | 0.47 |
| of biological process | | | | | | |
| (GO:0048519) | | | | | | |
| regulation of cellular | 0.47 | 0.47 | 0.47 | 0.47 | 0.47 | 0.47 |
| response to stress | | | | | | |
| (GO:0080135) | | | | | | |
| protein localization | 0.47 | 0.47 | 0.47 | 0.47 | 0.47 | 0.47 |
| (GO:0008104) | | | | | | |
| cell cycle | 0.47 | 0.47 | 0.47 | 0.47 | 0.47 | 0.47 |
| (GO:0007049) | | | | | | |
| regulation of | 0.47 | 0.47 | 0.47 | 0.47 | 0.47 | 0.47 |
| organelle | | | | | | |
| organization | | | | | | |
| (GO:0033043) | | | | | | |
| endocytosis | 0.47 | 0.47 | 0.47 | 0.47 | 0.47 | 0.47 |
| (GO:0006897) | | | | | | |
| extracellular matrix | 0.47 | 0.47 | 0.47 | 0.47 | 0.47 | 0.47 |
| disassembly | | | | | | |
| (GO:0022617) | | | | | | |
| anatomical structure | 0.47 | 0.47 | 0.47 | 0.47 | 0.47 | 0.47 |
| morphogenesis | | | | | | |
| (GO:0009653) | | | | | | |
| regulation of mitotic | 0.47 | 0.47 | 0.47 | 0.47 | 0.47 | 0.47 |
| metaphase/anaphase | | | | | | |
| transition | | | | | | |
| (GO:0030071) | | | | | | |
| negative regulation | 0.47 | 0.47 | 0.47 | 0.47 | 0.47 | 0.47 |
| of cell population | | | | | | |
| proliferation | | | | | | |
| (GO:0008285) | | | | | | |
| regulation of cell | 0.47 | 0.47 | 0.47 | 0.47 | 0.47 | 0.47 |
| cycle (GO:0051726) | | | | | | |
| cell cycle process | 0.47 | 0.47 | 0.47 | 0.47 | 0.47 | 0.47 |
| (GO:0022402) | | | | | | |
| extracellular matrix | 0.47 | 0.47 | 0.47 | 0.47 | 0.47 | 0.47 |
| assembly | | | | | | |
| (GO:0085029) | | | | | | |
| intracellular signal | 0.47 | 0.47 | 0.47 | 0.47 | 0.47 | 0.47 |
| transduction | | | | | | |
| (GO:0035556) | | | | | | |
| chromosome | 0.47 | 0.47 | 0.47 | 0.47 | 0.47 | 0.47 |
| organization | | | | | | |
| (GO:0051276) | | | | | | |
| transport along | 0.47 | 0.47 | 0.47 | 0.47 | 0.47 | 0.47 |
| microtubule | | | | | | |
| (GO:0010970) | | | | | | |
| embryo | 0.47 | 0.47 | 0.47 | 0.47 | 0.47 | 0.47 |
| development ending | | | | | | |
| in birth or egg | | | | | | |
| hatching | | | | | | |
| (GO:0009792) | | | | | | |
| protein | 0.47 | 0.47 | 0.47 | 0.47 | 0.47 | 0.47 |
| phosphorylation | | | | | | |
| (GO:0006468) | | | | | | |
| establishment of | 0.47 | 0.47 | 0.47 | 0.47 | 0.47 | 0.47 |
| localization | | | | | | |
| (GO:0051234) | | | | | | |
| nephron tubule | 0.47 | 0.47 | 0.47 | 0.47 | 0.47 | 0.47 |
| development | | | | | | |
| (GO:0072080) | | | | | | |
| regulation of cell | 0.47 | 0.47 | 0.47 | 0.47 | 0.47 | 0.47 |
| cycle process | | | | | | |
| (GO:0010564) | | | | | | |
| positive regulation | 0.47 | 0.47 | 0.47 | 0.47 | 0.47 | 0.47 |
| of signal | | | | | | |
| transduction | | | | | | |
| (GO:0009967) | | | | | | |
| regulation of | 0.47 | 0.47 | 0.47 | 0.47 | 0.47 | 0.47 |
| localization | | | | | | |
| (GO:0032879) | | | | | | |
| regulation of cellular | 0.47 | 0.47 | 0.47 | 0.47 | 0.47 | 0.47 |
| localization | | | | | | |
| (GO:0060341) | | | | | | |
| cytoskeleton | 0.47 | 0.47 | 0.47 | 0.47 | 0.47 | 0.47 |
| organization | | | | | | |
| (GO:0007010) | | | | | | |
| transport | 0.47 | 0.47 | 0.47 | 0.47 | 0.47 | 0.47 |
| (GO:0006810) | | | | | | |
| gland development | 0.47 | 0.47 | 0.47 | 0.47 | 0.47 | 0.47 |
| (GO:0048732) | | | | | | |
| establishment of | 0.47 | 0.47 | 0.47 | 0.47 | 0.47 | 0.47 |
| localization | | | | | | |
| (GO:0051234) | | | | | | |
| regulation of | 0.47 | 0.47 | 0.47 | 0.47 | 0.47 | 0.47 |
| chemotaxis | | | | | | |
| (GO:0050920) | | | | | | |
| nephron | 0.47 | 0.47 | 0.47 | 0.47 | 0.47 | 0.47 |
| development | | | | | | |
| (GO:0072006) | | | | | | |
| regulation of | 0.47 | 0.47 | 0.47 | 0.47 | 0.47 | 0.47 |
| phosphate metabolic | | | | | | |
| process | | | | | | |
| (GO:0019220) | | | | | | |
| regulation of | 0.47 | 0.47 | 0.47 | 0.47 | 0.47 | 0.47 |
| catalytic activity | | | | | | |
| (GO:0050790) | | | | | | |
| cell-substrate | 0.47 | 0.47 | 0.47 | 0.47 | 0.47 | 0.47 |
| adhesion | | | | | | |
| (GO:0031589) | | | | | | |
| macromolecule | 0.47 | 0.47 | 0.47 | 0.47 | 0.47 | 0.47 |
| modification | | | | | | |
| (GO:0043412) | | | | | | |
| small GTPase- | 0.47 | 0.47 | 0.47 | 0.47 | 0.47 | 0.47 |
| mediated signal | | | | | | |
| transduction | | | | | | |
| (GO:0007264) | | | | | | |
| positive regulation | 0.47 | 0.47 | 0.47 | 0.47 | 0.47 | 0.47 |
| of response to | | | | | | |
| external stimulus | | | | | | |
| (GO:0032103) | | | | | | |
| transport | 0.47 | 0.47 | 0.47 | 0.47 | 0.47 | 0.47 |
| (GO:0006810) | | | | | | |
| regulation of | 0.47 | 0.47 | 0.47 | 0.47 | 0.47 | 0.47 |
| primary metabolic | | | | | | |
| process | | | | | | |
| (GO:0080090) | | | | | | |
| multicellular | 0.47 | 0.47 | 0.46 | 0.47 | 0.47 | 0.47 |
| organism | | | | | | |
| development | | | | | | |
| (GO:0007275) | | | | | | |
| protein modification | 0.47 | 0.47 | 0.47 | 0.47 | 0.47 | 0.47 |
| process | | | | | | |
| (GO:0036211) | | | | | | |
| positive regulation | 0.47 | 0.47 | 0.47 | 0.47 | 0.47 | 0.47 |
| of cellular | | | | | | |
| biosynthetic process | | | | | | |
| (GO:0031328) | | | | | | |
| chordate embryonic | 0.47 | 0.47 | 0.47 | 0.47 | 0.47 | 0.47 |
| development | | | | | | |
| (GO:0043009) | | | | | | |
| positive regulation | 0.47 | 0.47 | 0.47 | 0.47 | 0.47 | 0.47 |
| of macromolecule | | | | | | |
| biosynthetic process | | | | | | |
| (GO:0010557) | | | | | | |
| localization | 0.47 | 0.47 | 0.47 | 0.47 | 0.47 | 0.47 |
| (GO:0051179) | | | | | | |
| positive regulation | 0.47 | 0.47 | 0.47 | 0.47 | 0.47 | 0.47 |
| of biosynthetic | | | | | | |
| process | | | | | | |
| (GO:0009891) | | | | | | |
| establishment of | 0.47 | 0.47 | 0.47 | 0.47 | 0.47 | 0.47 |
| organelle | | | | | | |
| localization | | | | | | |
| (GO:0051656) | | | | | | |
| vacuole organization | 0.47 | 0.47 | 0.47 | 0.47 | 0.47 | 0.47 |
| (GO:0007033) | | | | | | |
| export from cell | 0.47 | 0.47 | 0.47 | 0.47 | 0.47 | 0.47 |
| (GO:0140352) | | | | | | |
| positive regulation | 0.47 | 0.47 | 0.47 | 0.47 | 0.47 | 0.47 |
| of cell motility | | | | | | |
| (GO:2000147) | | | | | | |
| cellular response to | 0.47 | 0.47 | 0.47 | 0.47 | 0.47 | 0.47 |
| chemical stimulus | | | | | | |
| (GO:0070887) | | | | | | |
| embryo | 0.47 | 0.47 | 0.47 | 0.47 | 0.47 | 0.47 |
| development | | | | | | |
| (GO:0009790) | | | | | | |
| canonical Wnt | 0.47 | 0.47 | 0.47 | 0.47 | 0.47 | 0.47 |
| signaling pathway | | | | | | |
| (GO:0060070) | | | | | | |
| cellular localization | 0.47 | 0.47 | 0.47 | 0.47 | 0.47 | 0.47 |
| (GO:0051641) | | | | | | |
| glomerulus | 0.47 | 0.47 | 0.47 | 0.47 | 0.47 | 0.47 |
| development | | | | | | |
| (GO:0032835) | | | | | | |
| regulation of | 0.47 | 0.47 | 0.47 | 0.47 | 0.47 | 0.47 |
| vasculature | | | | | | |
| development | | | | | | |
| (GO:1901342) | | | | | | |
| ureteric bud | 0.47 | 0.47 | 0.47 | 0.47 | 0.47 | 0.47 |
| development | | | | | | |
| (GO:0001657) | | | | | | |
| signal transduction | 0.47 | 0.47 | 0.47 | 0.47 | 0.47 | 0.47 |
| (GO:0007165) | | | | | | |
| positive regulation | 0.47 | 0.47 | 0.47 | 0.47 | 0.47 | 0.47 |
| of protein | | | | | | |
| localization | | | | | | |
| (GO:1903829) | | | | | | |
| regulation of | 0.47 | 0.47 | 0.47 | 0.47 | 0.47 | 0.47 |
| endothelial cell | | | | | | |
| chemotaxis | | | | | | |
| (GO:2001026) | | | | | | |
| protein localization | 0.47 | 0.47 | 0.47 | 0.47 | 0.47 | 0.47 |
| to phagophore | | | | | | |
| assembly site | | | | | | |
| (GO:0034497) | | | | | | |
| regulation of | 0.47 | 0.47 | 0.47 | 0.47 | 0.47 | 0.47 |
| intracellular signal | | | | | | |
| transduction | | | | | | |
| (GO:1902531) | | | | | | |
| positive regulation | 0.47 | 0.47 | 0.47 | 0.47 | 0.47 | 0.47 |
| of response to | | | | | | |
| stimulus | | | | | | |
| GO:0048584) | | | | | | |
| nervous system | 0.47 | 0.47 | 0.47 | 0.47 | 0.47 | 0.47 |
| development | | | | | | |
| (GO:0007399) | | | | | | |
| regulation of | 0.47 | 0.47 | 0.47 | 0.47 | 0.47 | 0.47 |
| TORC1 signaling | | | | | | |
| (GO:1903432) | | | | | | |
| endothelial cell | 0.47 | 0.47 | 0.47 | 0.47 | 0.47 | 0.47 |
| proliferation | | | | | | |
| (GO:0001935) | | | | | | |
| mitotic cell cycle | 0.47 | 0.47 | 0.47 | 0.47 | 0.47 | 0.47 |
| (GO:0000278) | | | | | | |
| regulation of plasma | 0.47 | 0.47 | 0.47 | 0.47 | 0.47 | 0.47 |
| membrane bounded | | | | | | |
| cell projection | | | | | | |
| organization | | | | | | |
| (GO:0120035) | | | | | | |
| regulation of | 0.47 | 0.47 | 0.47 | 0.47 | 0.47 | 0.47 |
| synapse structure or | | | | | | |
| activity | | | | | | |
| (GO:0050803) | | | | | | |
| formation of primary | 0.47 | 0.47 | 0.47 | 0.47 | 0.47 | 0.47 |
| germ layer | | | | | | |
| (GO:0001704) | | | | | | |
| sensory organ | 0.47 | 0.47 | 0.47 | 0.47 | 0.47 | 0.47 |
| morphogenesis | | | | | | |
| (GO:0090596) | | | | | | |
| negative regulation | 0.47 | 0.47 | 0.47 | 0.47 | 0.47 | 0.47 |
| of transcription by | | | | | | |
| RNA polymerase II | | | | | | |
| (GO:0000122) | | | | | | |
| cellular anatomical | 0.47 | 0.47 | 0.47 | 0.47 | 0.47 | 0.47 |
| entity | | | | | | |
| morphogenesis | | | | | | |
| (GO:0032989) | | | | | | |
| regulation of | 0.47 | 0.47 | 0.47 | 0.47 | 0.47 | 0.47 |
| macromolecule | | | | | | |
| metabolic process | | | | | | |
| (GO:0060255) | | | | | | |
| embryonic | 0.47 | 0.47 | 0.47 | 0.47 | 0.47 | 0.47 |
| morphogenesis | | | | | | |
| (GO:0048598) | | | | | | |
| regulation of | 0.47 | 0.47 | 0.47 | 0.47 | 0.47 | 0.47 |
| osteoblast | | | | | | |
| differentiation | | | | | | |
| (GO:0045667) | | | | | | |
| cellular response to | 0.47 | 0.47 | 0.47 | 0.47 | 0.47 | 0.47 |
| transforming growth | | | | | | |
| factor beta stimulus | | | | | | |
| (GO:0071560) | | | | | | |
| positive regulation | 0.47 | 0.47 | 0.47 | 0.47 | 0.47 | 0.47 |
| of cellular | | | | | | |
| component | | | | | | |
| biogenesis | | | | | | |
| (GO:0044089) | | | | | | |
| organelle | 0.47 | 0.47 | 0.47 | 0.47 | 0.47 | 0.47 |
| organization | | | | | | |
| (GO:0006996) | | | | | | |
| anatomical structure | 0.47 | 0.47 | 0.47 | 0.47 | 0.47 | 0.47 |
| development | | | | | | |
| (GO:0048856) | | | | | | |
| mesenchymal cell | 0.47 | 0.47 | 0.47 | 0.47 | 0.46 | 0.47 |
| differentiation | | | | | | |
| (GO:0048762) | | | | | | |
| regulation of mitotic | 0.47 | 0.47 | 0.47 | 0.47 | 0.47 | 0.47 |
| cell cycle | | | | | | |
| (GO:0007346) | | | | | | |
| mitotic sister | 0.47 | 0.47 | 0.47 | 0.47 | 0.47 | 0.47 |
| chromatid | | | | | | |
| segregation | | | | | | |
| (GO:0000070) | | | | | | |
| regulation of | 0.46 | 0.47 | 0.46 | 0.47 | 0.47 | 0.47 |
| signaling | | | | | | |
| (GO:0023051) | | | | | | |
| protein metabolic | 0.47 | 0.47 | 0.47 | 0.47 | 0.47 | 0.47 |
| process | | | | | | |
| (GO:0019538) | | | | | | |
| eye development | 0.47 | 0.47 | 0.47 | 0.47 | 0.47 | 0.47 |
| (GO:0001654) | | | | | | |
| neurogenesis | 0.47 | 0.47 | 0.47 | 0.47 | 0.47 | 0.47 |
| (GO:0022008) | | | | | | |
| endochondral bone | 0.47 | 0.47 | 0.47 | 0.47 | 0.47 | 0.47 |
| morphogenesis | | | | | | |
| (GO:0060350) | | | | | | |
| regulation of | 0.47 | 0.47 | 0.47 | 0.47 | 0.47 | 0.47 |
| synapse organization | | | | | | |
| (GO:0050807) | | | | | | |
| regulation of | 0.47 | 0.47 | 0.47 | 0.47 | 0.47 | 0.47 |
| collagen metabolic | | | | | | |
| process | | | | | | |
| (GO:0010712) | | | | | | |
| cellular response to | 0.47 | 0.47 | 0.47 | 0.47 | 0.47 | 0.47 |
| endogenous stimulus | | | | | | |
| (GO:0071495) | | | | | | |
| regulation of | 0.47 | 0.47 | 0.47 | 0.47 | 0.47 | 0.47 |
| catabolic process | | | | | | |
| (GO:0009894) | | | | | | |
| cartilage | 0.47 | 0.47 | 0.47 | 0.47 | 0.46 | 0.46 |
| development | | | | | | |
| (GO:0051216) | | | | | | |
| cell communication | 0.47 | 0.47 | 0.46 | 0.47 | 0.47 | 0.47 |
| (GO:0007154) | | | | | | |
| mesenchyme | 0.47 | 0.47 | 0.47 | 0.47 | 0.47 | 0.47 |
| development | | | | | | |
| (GO:0060485) | | | | | | |
| localization | 0.47 | 0.47 | 0.46 | 0.47 | 0.47 | 0.47 |
| (GO:0051179) | | | | | | |
| regulation of | 0.47 | 0.47 | 0.46 | 0.47 | 0.47 | 0.47 |
| metabolic process | | | | | | |
| (GO:0019222) | | | | | | |
| regulation of cellular | 0.47 | 0.47 | 0.47 | 0.47 | 0.47 | 0.47 |
| catabolic process | | | | | | |
| (GO:0031329) | | | | | | |
| programmed cell | 0.47 | 0.47 | 0.46 | 0.47 | 0.46 | 0.47 |
| death (GO:0012501) | | | | | | |
| endomembrane | 0.47 | 0.47 | 0.46 | 0.47 | 0.46 | 0.47 |
| system organization | | | | | | |
| (GO:0010256) | | | | | | |
| protein modification | 0.47 | 0.47 | 0.47 | 0.47 | 0.46 | 0.47 |
| by small protein | | | | | | |
| conjugation or | | | | | | |
| removal | | | | | | |
| (GO:0070647) | | | | | | |
| endodennal cell | 0.47 | 0.47 | 0.47 | 0.47 | 0.47 | 0.46 |
| differentiation | | | | | | |
| (GO:0035987) | | | | | | |
| positive regulation | 0.47 | 0.47 | 0.47 | 0.47 | 0.46 | 0.47 |
| of organelle | | | | | | |
| organization | | | | | | |
| (GO:0010638) | | | | | | |
| Wnt signaling | 0.47 | 0.47 | 0.47 | 0.47 | 0.47 | 0.47 |
| pathway | | | | | | |
| (GO:0016055) | | | | | | |
| sprouting | 0.47 | 0.47 | 0.47 | 0.47 | 0.46 | 0.46 |
| angiogenesis | | | | | | |
| (GO:0002040) | | | | | | |
| cellular response to | 0.47 | 0.47 | 0.47 | 0.47 | 0.47 | 0.47 |
| growth factor | | | | | | |
| stimulus | | | | | | |
| (GO:0071363) | | | | | | |
| mitotic chromosome | 0.47 | 0.47 | 0.46 | 0.47 | 0.47 | 0.47 |
| condensation | | | | | | |
| (GO:0007076) | | | | | | |
| muscle structure | 0.47 | 0.47 | 0.47 | 0.47 | 0.47 | 0.47 |
| development | | | | | | |
| (GO:0061061) | | | | | | |
| developmental | 0.47 | 0.47 | 0.46 | 0.47 | 0.46 | 0.47 |
| process | | | | | | |
| (GO:0032502) | | | | | | |
| DNA metabolic | 0.47 | 0.47 | 0.47 | 0.47 | 0.47 | 0.47 |
| process | | | | | | |
| (GO:0006259) | | | | | | |
| regulation of DNA- | 0.47 | 0.47 | 0.46 | 0.47 | 0.46 | 0.47 |
| templated | | | | | | |
| transcription | | | | | | |
| (GO:0006355) | | | | | | |
| collagen | 0.47 | 0.47 | 0.47 | 0.47 | 0.47 | 0.47 |
| biosynthetic process | | | | | | |
| (GO:0032964) | | | | | | |
| regulation of RNA | 0.47 | 0.47 | 0.46 | 0.47 | 0.46 | 0.47 |
| metabolic process | | | | | | |
| (GO:0051252) | | | | | | |
| anatomical structure | 0.47 | 0.47 | 0.47 | 0.47 | 0.46 | 0.46 |
| formation involved | | | | | | |
| in morphogenesis | | | | | | |
| (GO:0048646) | | | | | | |
| regulation of RNA | 0.47 | 0.47 | 0.46 | 0.47 | 0.46 | 0.47 |
| biosynthetic process | | | | | | |
| (GO:2001141) | | | | | | |
| regulation of | 0.47 | 0.47 | 0.46 | 0.47 | 0.46 | 0.47 |
| transcription by | | | | | | |
| RNA polymerase II | | | | | | |
| (GO:0006357) | | | | | | |
| cellular nitrogen | 0.47 | 0.47 | 0.46 | 0.47 | 0.46 | 0.47 |
| compound | | | | | | |
| biosynthetic process | | | | | | |
| (GO:0044271) | | | | | | |
| cytoskeleton- | 0.47 | 0.47 | 0.47 | 0.47 | 0.47 | 0.46 |
| dependent | | | | | | |
| intracellular | | | | | | |
| transport | | | | | | |
| (GO:0030705) | | | | | | |
| negative regulation | 0.47 | 0.47 | 0.47 | 0.47 | 0.46 | 0.46 |
| of developmental | | | | | | |
| process | | | | | | |
| (GO:0051093) | | | | | | |
| transcription by | 0.47 | 0.47 | 0.46 | 0.47 | 0.46 | 0.47 |
| RNA polymerase II | | | | | | |
| (GO:0006366) | | | | | | |
| regulation of | 0.47 | 0.47 | 0.46 | 0.47 | 0.46 | 0.47 |
| biological quality | | | | | | |
| (GO:0065008) | | | | | | |
| epithelium | 0.47 | 0.47 | 0.47 | 0.47 | 0.46 | 0.46 |
| development | | | | | | |
| (GO:0060429) | | | | | | |
| vesicle-mediated | 0.47 | 0.47 | 0.46 | 0.47 | 0.47 | 0.46 |
| transport | | | | | | |
| (GO:0016192) | | | | | | |
| organic cyclic | 0.47 | 0.47 | 0.46 | 0.47 | 0.46 | 0.47 |
| compound | | | | | | |
| biosynthetic process | | | | | | |
| (GO:1901362) | | | | | | |
| aromatic compound | 0.47 | 0.47 | 0.46 | 0.47 | 0.46 | 0.47 |
| biosynthetic process | | | | | | |
| (GO:0019438) | | | | | | |
| meiotic cell cycle | 0.47 | 0.47 | 0.46 | 0.46 | 0.46 | 0.46 |
| process | | | | | | |
| (GO:1903046) | | | | | | |
| heterocycle | 0.47 | 0.47 | 0.46 | 0.47 | 0.46 | 0.47 |
| biosynthetic process | | | | | | |
| (GO:0018130) | | | | | | |
| skeletal system | 0.47 | 0.47 | 0.46 | 0.47 | 0.46 | 0.46 |
| development | | | | | | |
| (GO:0001501) | | | | | | |
| positive regulation | 0.47 | 0.47 | 0.46 | 0.47 | 0.46 | 0.47 |
| of cellular | | | | | | |
| component | | | | | | |
| organization | | | | | | |
| (GO:0051130) | | | | | | |
| endodenn | 0.46 | 0.47 | 0.47 | 0.47 | 0.47 | 0.46 |
| development | | | | | | |
| (GO:0007492) | | | | | | |
| nucleobase- | 0.47 | 0.47 | 0.46 | 0.46 | 0.46 | 0.47 |
| containing | | | | | | |
| compound | | | | | | |
| biosynthetic process | | | | | | |
| (GO:0034654) | | | | | | |
| system development | 0.47 | 0.47 | 0.46 | 0.47 | 0.46 | 0.46 |
| (GO:0048731) | | | | | | |
| vasculature | 0.46 | 0.47 | 0.47 | 0.47 | 0.46 | 0.46 |
| development | | | | | | |
| (GO:0001944) | | | | | | |
| negative regulation | 0.47 | 0.47 | 0.46 | 0.47 | 0.46 | 0.46 |
| of biological process | | | | | | |
| (GO:0048519) | | | | | | |
| blood vessel | 0.46 | 0.47 | 0.46 | 0.47 | 0.46 | 0.46 |
| morphogenesis | | | | | | |
| (GO:0048514) | | | | | | |
| regulation of | 0.46 | 0.47 | 0.46 | 0.47 | 0.46 | 0.46 |
| macroautophagy | | | | | | |
| (GO:0016241) | | | | | | |
| DNA-templated | 0.47 | 0.47 | 0.46 | 0.46 | 0.46 | 0.47 |
| transcription | | | | | | |
| (GO:0006351) | | | | | | |
| DNA damage | 0.47 | 0.46 | 0.46 | 0.46 | 0.46 | 0.47 |
| response | | | | | | |
| (GO:0006974) | | | | | | |
| biological regulation | 0.47 | 0.47 | 0.46 | 0.47 | 0.46 | 0.47 |
| (GO:0065007) | | | | | | |
| RNA biosynthetic | 0.47 | 0.47 | 0.46 | 0.46 | 0.46 | 0.47 |
| process | | | | | | |
| (GO:0032774) | | | | | | |
| regulation of | 0.47 | 0.47 | 0.46 | 0.47 | 0.46 | 0.47 |
| biological process | | | | | | |
| (GO:0050789) | | | | | | |
| regulation of cellular | 0.46 | 0.47 | 0.46 | 0.46 | 0.46 | 0.46 |
| response to growth | | | | | | |
| factor stimulus | | | | | | |
| (GO:0090287) | | | | | | |
| regulation of cellular | 0.47 | 0.47 | 0.46 | 0.46 | 0.46 | 0.47 |
| process | | | | | | |
| (GO:0050794) | | | | | | |
| tube development | 0.46 | 0.47 | 0.46 | 0.46 | 0.46 | 0.46 |
| (GO:0035295) | | | | | | |
| multicellular | 0.47 | 0.47 | 0.46 | 0.46 | 0.46 | 0.46 |
| organism | | | | | | |
| development | | | | | | |
| (GO:0007275) | | | | | | |
| positive regulation | 0.46 | 0.47 | 0.46 | 0.46 | 0.46 | 0.47 |
| of catabolic process | | | | | | |
| (GO:0009896) | | | | | | |
| regulation of cellular | 0.46 | 0.46 | 0.46 | 0.47 | 0.46 | 0.47 |
| component | | | | | | |
| biogenesis | | | | | | |
| (GO:0044087) | | | | | | |
| actin filament-based | 0.47 | 0.47 | 0.46 | 0.46 | 0.46 | 0.46 |
| process | | | | | | |
| (GO:0030029) | | | | | | |
| actin cytoskeleton | 0.47 | 0.47 | 0.46 | 0.46 | 0.46 | 0.46 |
| organization | | | | | | |
| (GO:0030036) | | | | | | |
| artery | 0.46 | 0.47 | 0.47 | 0.46 | 0.46 | 0.46 |
| morphogenesis | | | | | | |
| (GO:0048844) | | | | | | |
| regulation of gene | 0.47 | 0.47 | 0.46 | 0.46 | 0.46 | 0.47 |
| expression | | | | | | |
| (GO:0010468) | | | | | | |
| heart development | 0.46 | 0.46 | 0.46 | 0.46 | 0.46 | 0.46 |
| (GO:0007507) | | | | | | |
| regulation of cellular | 0.46 | 0.47 | 0.46 | 0.46 | 0.46 | 0.47 |
| metabolic process | | | | | | |
| (GO:0031323) | | | | | | |
| transforming growth | 0.46 | 0.47 | 0.46 | 0.46 | 0.46 | 0.46 |
| factor beta receptor | | | | | | |
| superfamily | | | | | | |
| signaling pathway | | | | | | |
| (GO:0141091) | | | | | | |
| cell junction | 0.47 | 0.46 | 0.46 | 0.47 | 0.46 | 0.46 |
| organization | | | | | | |
| (GO:0034330) | | | | | | |
| catabolic process | 0.46 | 0.46 | 0.46 | 0.46 | 0.46 | 0.46 |
| (GO:0009056) | | | | | | |
| chondrocyte | 0.46 | 0.46 | 0.46 | 0.46 | 0.46 | 0.46 |
| differentiation | | | | | | |
| (GO:0002062) | | | | | | |
| organonitrogen | 0.47 | 0.46 | 0.46 | 0.46 | 0.46 | 0.46 |
| compound metabolic | | | | | | |
| process | | | | | | |
| (GO:1901564) | | | | | | |
| transmembrane | 0.46 | 0.47 | 0.46 | 0.46 | 0.46 | 0.46 |
| receptor protein | | | | | | |
| serine/threonine | | | | | | |
| kinase signaling | | | | | | |
| pathway | | | | | | |
| (GO:0007178) | | | | | | |
| TORC1 signaling | 0.46 | 0.46 | 0.46 | 0.46 | 0.46 | 0.47 |
| (GO:0038202) | | | | | | |
| regulation of | 0.46 | 0.46 | 0.46 | 0.46 | 0.46 | 0.46 |
| developmental | | | | | | |
| process | | | | | | |
| (GO:0050793) | | | | | | |
| response to stimulus | 0.47 | 0.47 | 0.46 | 0.46 | 0.46 | 0.46 |
| (GO:0050896) | | | | | | |
| MAPK cascade | 0.46 | 0.47 | 0.46 | 0.46 | 0.46 | 0.46 |
| (GO:0000165) | | | | | | |
| negative regulation | 0.46 | 0.46 | 0.45 | 0.47 | 0.46 | 0.46 |
| of cellular process | | | | | | |
| (GO:0048523) | | | | | | |
| regulation of DNA | 0.46 | 0.46 | 0.46 | 0.46 | 0.46 | 0.46 |
| metabolic process | | | | | | |
| (GO:0051052) | | | | | | |
| signal transduction | 0.46 | 0.47 | 0.45 | 0.47 | 0.46 | 0.46 |
| (GO:0007165) | | | | | | |
| positive regulation | 0.46 | 0.46 | 0.46 | 0.46 | 0.46 | 0.46 |
| of autophagy | | | | | | |
| (GO:0010508) | | | | | | |
| intracellular | 0.46 | 0.46 | 0.46 | 0.46 | 0.46 | 0.46 |
| signaling cassette | | | | | | |
| (GO:0141124) | | | | | | |
| regulation of MAPK | 0.46 | 0.47 | 0.46 | 0.46 | 0.45 | 0.46 |
| cascade | | | | | | |
| (GO:0043408) | | | | | | |
| regulation of cellular | 0.46 | 0.46 | 0.46 | 0.46 | 0.46 | 0.46 |
| component size | | | | | | |
| (GO:0032535) | | | | | | |
| regulation of cell | 0.46 | 0.46 | 0.46 | 0.46 | 0.46 | 0.46 |
| size (GO:0008361) | | | | | | |
| regulation of protein | 0.46 | 0.46 | 0.46 | 0.46 | 0.46 | 0.46 |
| localization | | | | | | |
| (GO:0032880) | | | | | | |
| cell projection | 0.46 | 0.47 | 0.46 | 0.46 | 0.46 | 0.46 |
| organization | | | | | | |
| (GO:0030030) | | | | | | |
| regulation of plasma | 0.46 | 0.46 | 0.46 | 0.46 | 0.46 | 0.46 |
| membrane bounded | | | | | | |
| cell projection | | | | | | |
| assembly | | | | | | |
| (GO:0120032) | | | | | | |
| organic substance | 0.46 | 0.46 | 0.46 | 0.46 | 0.45 | 0.46 |
| catabolic process | | | | | | |
| (GO:1901575) | | | | | | |
| signaling | 0.46 | 0.46 | 0.45 | 0.46 | 0.46 | 0.46 |
| (GO:0023052) | | | | | | |
| morphogenesis of an | 0.46 | 0.47 | 0.46 | 0.46 | 0.45 | 0.45 |
| epithelium | | | | | | |
| (GO:0002009) | | | | | | |
| regulation of | 0.47 | 0.47 | 0.46 | 0.47 | 0.46 | 0.43 |
| nucleobase- | | | | | | |
| containing | | | | | | |
| compound metabolic | | | | | | |
| process | | | | | | |
| (GO:0019219) | | | | | | |
| lung development | 0.46 | 0.47 | 0.46 | 0.46 | 0.46 | 0.45 |
| (GO:0030324) | | | | | | |
| protein transport | 0.46 | 0.46 | 0.45 | 0.46 | 0.46 | 0.46 |
| (GO:0015031) | | | | | | |
| mesenchymal cell | 0.46 | 0.46 | 0.46 | 0.46 | 0.45 | 0.46 |
| proliferation | | | | | | |
| (GO:0010463) | | | | | | |
| proteolysis | 0.46 | 0.46 | 0.46 | 0.46 | 0.45 | 0.46 |
| (GO:0006508) | | | | | | |
| cell communication | 0.46 | 0.46 | 0.45 | 0.46 | 0.46 | 0.46 |
| (GO:0007154) | | | | | | |
| tongue development | 0.46 | 0.46 | 0.46 | 0.46 | 0.46 | 0.46 |
| (GO:0006950) | | | | | | |
| positive regulation | 0.44 | 0.46 | 0.45 | 0.46 | 0.46 | 0.45 |
| of establishment of | | | | | | |
| protein localization | | | | | | |
| (GO:1904951) | | | | | | |
| autophagy | 0.45 | 0.45 | 0.45 | 0.45 | 0.46 | 0.45 |
| (GO:0006914) | | | | | | |
| intracellular | 0.45 | 0.46 | 0.44 | 0.46 | 0.45 | 0.45 |
| signaling cassette | | | | | | |
| (GO:0141124) | | | | | | |
| organonitrogen | 0.45 | 0.45 | 0.45 | 0.46 | 0.44 | 0.46 |
| compound catabolic | | | | | | |
| process | | | | | | |
| (GO:1901565) | | | | | | |
| protein catabolic | 0.45 | 0.45 | 0.45 | 0.46 | 0.44 | 0.45 |
| process | | | | | | |
| (GO:0030163) | | | | | | |
| positive regulation | 0.47 | 0.45 | 0.42 | 0.48 | 0.36 | 0.47 |
| of transport | | | | | | |
| (GO:0051050) | | | | | | |
| regulation of cell | 0.41 | 0.46 | 0.39 | 0.47 | 0.41 | 0.46 |
| communication | | | | | | |
| GO:0010646) | | | | | | |
| regulation of | 0.46 | 0.47 | 0.35 | 0.47 | 0.40 | 0.42 |
| nitrogen compound | | | | | | |
| metabolic process | | | | | | |
| (GO:0051171) | | | | | | |
| regulation of | 0.48 | 0.47 | 0.23 | 0.48 | 0.44 | 0.46 |
| primary metabolic | | | | | | |
| process | | | | | | |
| (GO:0080090) | | | | | | |
| cell development | 0.48 | 0.48 | 0.34 | 0.48 | 0.34 | 0.42 |
| (GO:0048468) | | | | | | |
| protein modification | 0.42 | 0.47 | 0.38 | 0.46 | 0.44 | 0.35 |
| process | | | | | | |
| GO:0036211) | | | | | | |
| macromolecule | 0.45 | 0.48 | 0.33 | 0.48 | 0.43 | 0.33 |
| modification | | | | | | |
| (GO:0043412) | | | | | | |
| anatomical structure | 0.35 | 0.47 | 0.46 | 0.33 | 0.46 | 0.42 |
| development | | | | | | |
| (GO:0048856) | | | | | | |
| positive regulation | 0.42 | 0.44 | 0.36 | 0.42 | 0.40 | 0.44 |
| of protein | | | | | | |
| localization | | | | | | |
| (GO:1903829) | | | | | | |
| protein localization | 0.48 | 0.34 | 0.34 | 0.48 | 0.34 | 0.48 |
| to plasma membrane | | | | | | |
| (GO:0072659) | | | | | | |
| intracellular signal | 0.47 | 0.48 | 0.06 | 0.48 | 0.47 | 0.38 |
| transduction | | | | | | |
| (GO:0035556) | | | | | | |
| positive regulation | 0.31 | 0.47 | 0.28 | 0.48 | 0.28 | 0.48 |
| of intracellular | | | | | | |
| signal transduction | | | | | | |
| (GO:1902533) | | | | | | |
| ubiquitin-dependent | 0.45 | 0.27 | 0.30 | 0.47 | 0.27 | 0.27 |
| protein catabolic | | | | | | |
| process | | | | | | |
| (GO:0006511) | | | | | | |

### Example 3

### Lung Adenocarcinoma (LUAD), Ovarian Clear Cell Carcinoma (OCCC), Myxofibrosarcoma (MFS)

In one embodiment, this invention provides a method for identifying dysregulated gene sets and pathways. Tumor tissue samples are obtained, and RNA sequencing is conducted using Next Generation Sequencing (NGS). The resulting RNA sequencing data undergo preprocessing before being input into the machine learning model (MLM) for the identification of chemotherapy resistance risk and prioritization of dysregulated pathways. Each pathway within the established MLM is assigned a dysregulated pathway (DP) score, calculated as the first-layer probability multiplied by the second-layer weights. Pathways with the highest DP scores are considered the most significant contributors to state-associated risk. The Shapley Additive Explanations (SHAP) method is employed to identify the genes that most significantly influence the high-probability predictions. The gene with the highest positive contribution to predicting resistance is then matched with an appropriate FDA-approved targeted therapy.

| | LUAD 1 | LUAD 2 | OCCC | MFS 1 | MFS 2 |
|---|---|---|---|---|---|
| anatomical structure | 0.608442 | 0.60912 | 0.60731 | 0.637722 | 0.64651 |
| development | | | | | |
| (GO:0048856) | | | | | |
| extracellular matrix | 0.593842 | 0.588487 | 0.570225 | 0.673488 | 0.658667 |
| organization | | | | | |
| (GO:0030198) | | | | | |
| nephron epithelium | 0.613635 | 0.613626 | 0.614476 | 0.654737 | 0.653068 |
| development | | | | | |
| (GO:0072009) | | | | | |
| positive regulation | 0.615103 | 0.615099 | 0.61592 | 0.574687 | 0.579291 |
| of epithelial cell | | | | | |
| proliferation | | | | | |
| (GO:0050679) | | | | | |
| regulation of | 0.649175 | 0.667924 | 0.679994 | 0.651118 | 0.655532 |
| cellular component | | | | | |
| organization | | | | | |
| (GO:0051128) | | | | | |
| programmed cell | 0.613789 | 0.613785 | 0.614621 | 0.632531 | 0.666247 |
| death | | | | | |
| (GO:0012501) | | | | | |
| sensory organ | 0.612704 | 0.612704 | 0.613596 | 0.232064 | 0.650662 |
| development | | | | | |
| (GO:0007423) | | | | | |
| Wnt signaling | 0.620119 | 0.622198 | 0.631401 | 0.665189 | 0.659433 |
| pathway | | | | | |
| (GO:0016055) | | | | | |
| endothelial cell | 0.671456 | 0.677921 | 0.743176 | 0.716858 | 0.69053 |
| proliferation | | | | | |
| (GO:0001935) | | | | | |
| cellular response to | 0.642818 | 0.626028 | 0.646356 | 0.66778 | 0.667402 |
| organic substance | | | | | |
| (GO:0071310) | | | | | |
| epithelium | 0.587375 | 0.582722 | 0.569626 | 0.612847 | 0.622047 |
| development | | | | | |
| (GO:0060429) | | | | | |
| apoptotic process | 0.613907 | 0.613901 | 0.614737 | 0.657493 | 0.639387 |
| (GO:0006915) | | | | | |
| cell migration | 0.591395 | 0.586802 | 0.590093 | 0.60441 | 0.626843 |
| (GO:0016477) | | | | | |
| actin cytoskeleton | 0.612267 | 0.612272 | 0.613118 | 0.644237 | 0.625483 |
| organization | | | | | |
| (GO:0030036) | | | | | |
| regulation of | 0.572688 | 0.562105 | 0.559411 | 0.620673 | 0.646846 |
| angiogenesis | | | | | |
| (GO:0045765) | | | | | |
| positive regulation | 0.643316 | 0.65814 | 0.691556 | 0.64174 | 0.736312 |
| of angiogenesis | | | | | |
| (GO:0045766) | | | | | |
| neurogenesis | 0.613124 | 0.613123 | 0.61397 | 0.581479 | 0.620969 |
| (GO:0022008) | | | | | |
| response to | 0.614773 | 0.614754 | 0.615589 | 0.631444 | 0.655056 |
| wounding | | | | | |
| (GO:0009611) | | | | | |
| regulation of | 0.567393 | 0.568494 | 0.554969 | 0.64335 | 0.646598 |
| programmed cell | | | | | |
| death | | | | | |
| (GO:0043067) | | | | | |
| bone development | 0.568763 | 0.554292 | 0.559009 | 0.619411 | 0.615221 |
| (GO:0060348) | | | | | |
| negative regulation | 0.612546 | 0.612553 | 0.613421 | 0.642434 | 0.670609 |
| of locomotion | | | | | |
| (GO:0040013) | | | | | |
| negative regulation | 0.614809 | 0.614798 | 0.615632 | 0.521704 | 0.553599 |
| of cell | | | | | |
| differentiation | | | | | |
| (GO:0045596) | | | | | |
| chondrocyte | 0.61277 | 0.61277 | 0.613658 | 0.665921 | 0.654872 |
| differentiation | | | | | |
| (GO:0002062) | | | | | |
| vasculature | 0.61408 | 0.614074 | 0.614917 | 0.599611 | 0.625262 |
| development | | | | | |
| (GO:0001944) | | | | | |
| response to oxygen- | 0.612531 | 0.61253 | 0.613379 | 0.659968 | 0.677163 |
| containing | | | | | |
| compound | | | | | |
| (GO:1901700) | | | | | |
| kidney epithelium | 0.613707 | 0.613699 | 0.614545 | 0.675574 | 0.673296 |
| development | | | | | |
| (GO:0072073) | | | | | |
| regulation of | 0.57514 | 0.57431 | 0.563614 | 0.611916 | 0.630045 |
| apoptotic process | | | | | |
| (GO:0042981) | | | | | |
| regulation of | 0.612819 | 0.612819 | 0.613709 | 0.618937 | 0.623498 |
| transmembrane | | | | | |
| receptor protein | | | | | |
| serine/threonine | | | | | |
| kinase signaling | | | | | |
| pathway | | | | | |
| (GO:0090092) | | | | | |
| renal tubule | 0.613392 | 0.613388 | 0.614235 | 0.651928 | 0.680006 |
| morphogenesis | | | | | |
| (GO:0061333) | | | | | |
| negative regulation | 0.526387 | 0.534607 | 0.413354 | 0.480563 | 0.596765 |
| of multicellular | | | | | |
| organismal process | | | | | |
| (GO:0051241) | | | | | |
| regulation of | 0.611498 | 0.611514 | 0.612367 | 0.613255 | 0.61867 |
| cellular component | | | | | |
| biogenesis | | | | | |
| (GO:0044087) | | | | | |
| kidney | 0.613789 | 0.613776 | 0.614609 | 0.668092 | 0.679263 |
| morphogenesis | | | | | |
| (GO:0060993) | | | | | |
| regulation of | 0.612518 | 0.61252 | 0.613369 | 0.660266 | 0.650331 |
| molecular function | | | | | |
| (GO:0065009) | | | | | |
| tissue migration | 0.566129 | 0.555022 | 0.560104 | 0.551935 | 0.622777 |
| (GO:0090130) | | | | | |
| tube development | 0.614068 | 0.614055 | 0.614906 | 0.583538 | 0.631288 |
| (GO:0035295) | | | | | |
| collagen metabolic | 0.612847 | 0.612847 | 0.613735 | 0.608804 | 0.651762 |
| process | | | | | |
| (GO:0032963) | | | | | |
| negative regulation | 0.613068 | 0.613067 | 0.613901 | 0.65065 | 0.631014 |
| of signal | | | | | |
| transduction | | | | | |
| (GO:0009968) | | | | | |
| embryonic | 0.577199 | 0.571287 | 0.532513 | 0.625246 | 0.616488 |
| morphogenesis | | | | | |
| (GO:0048598) | | | | | |
| transforming | 0.6128 | 0.6128 | 0.613691 | 0.627023 | 0.639923 |
| growth factor beta | | | | | |
| receptor | | | | | |
| superfamily | | | | | |
| signaling pathway | | | | | |
| (GO:0141091) | | | | | |
| protein metabolic | 0.654386 | 0.663223 | 0.681691 | 0.649044 | 0.700225 |
| process | | | | | |
| (GO:0019538) | | | | | |
| generation of | 0.613615 | 0.613609 | 0.614453 | 0.621587 | 0.65086 |
| neurons | | | | | |
| (GO:0048699) | | | | | |
| regulation of | 0.595422 | 0.586143 | 0.584168 | 0.624385 | 0.665615 |
| phosphate | | | | | |
| metabolic process | | | | | |
| (GO:0019220) | | | | | |
| negative regulation | 0.615014 | 0.617256 | 0.616592 | 0.646953 | 0.662417 |
| of cell motility | | | | | |
| (GO:2000146) | | | | | |
| morphogenesis of | 0.59266 | 0.576244 | 0.580488 | 0.636891 | 0.668186 |
| an epithelium | | | | | |
| (GO:0002009) | | | | | |
| mesonephros | 0.61476 | 0.614736 | 0.615533 | 0.600055 | 0.590848 |
| development | | | | | |
| (GO:0001823) | | | | | |
| cell adhesion | 0.614523 | 0.614506 | 0.615352 | 0.605941 | 0.617521 |
| (GO:0007155) | | | | | |
| epithelial cell | 0.614421 | 0.614402 | 0.615232 | 0.332137 | 0.68074 |
| migration | | | | | |
| (GO:0010631) | | | | | |
| canonical Wnt | 0.612628 | 0.612628 | 0.613517 | 0.711606 | 0.704227 |
| signaling pathway | | | | | |
| (GO:0060070) | | | | | |
| nephron tubule | 0.645563 | 0.653685 | 0.653621 | 0.642488 | 0.665859 |
| morphogenesis | | | | | |
| (GO:0072078) | | | | | |
| negative regulation | 0.621396 | 0.630172 | 0.627665 | 0.66082 | 0.670979 |
| of cell migration | | | | | |
| (GO:0030336) | | | | | |
| negative regulation | 0.580199 | 0.56574 | 0.570241 | 0.60904 | 0.60408 |
| of cell adhesion | | | | | |
| (GO:0007162) | | | | | |
| cell junction | 0.639931 | 0.653787 | 0.657879 | 0.687701 | 0.697726 |
| organization | | | | | |
| (GO:0034330) | | | | | |
| head development | 0.605376 | 0.603025 | 0.598552 | 0.633476 | 0.64671 |
| (GO:0060322) | | | | | |
| central nervous | 0.612722 | 0.612723 | 0.613615 | 0.567108 | 0.602461 |
| system | | | | | |
| development | | | | | |
| (GO:0007417) | | | | | |
| metanephros | 0.612778 | 0.612778 | 0.613666 | 0.586759 | 0.393738 |
| development | | | | | |
| (GO:0001656) | | | | | |
| response to | 0.639545 | 0.628457 | 0.647872 | 0.68831 | 0.664727 |
| chemical | | | | | |
| (GO:0042221) | | | | | |
| tube morphogenesis | 0.613912 | 0.6139 | 0.614749 | 0.590494 | 0.628107 |
| (GO:0035239) | | | | | |
| transmembrane | 0.612813 | 0.612813 | 0.613704 | 0.620872 | 0.634543 |
| receptor protein | | | | | |
| serine/threonine | | | | | |
| kinase signaling | | | | | |
| pathway | | | | | |
| (GO:0007178) | | | | | |
| positive regulation | 0.61453 | 0.614509 | 0.615352 | 0.567329 | 0.621293 |
| of cell-substrate | | | | | |
| adhesion | | | | | |
| (GO:0010811) | | | | | |
| cytoskeleton | 0.611655 | 0.611655 | 0.612513 | 0.634115 | 0.613315 |
| organization | | | | | |
| (GO:0007010) | | | | | |
| embryo | 0.636169 | 0.646751 | 0.652825 | 0.620526 | 0.641409 |
| development | | | | | |
| (GO:0009790) | | | | | |
| positive regulation | 0.613942 | 0.613938 | 0.614789 | 0.600302 | 0.767783 |
| of multicellular | | | | | |
| organismal process | | | | | |
| (GO:0051240) | | | | | |
| positive regulation | 0.614386 | 0.614373 | 0.615222 | 0.589354 | 0.620567 |
| of cell population | | | | | |
| proliferation | | | | | |
| (GO:0008284) | | | | | |
| muscle organ | 0.612741 | 0.612741 | 0.613631 | 0.819445 | 0.656135 |
| development | | | | | |
| (GO:0007517) | | | | | |
| mesonephric tubule | 0.615121 | 0.61509 | 0.615889 | 0.618446 | 0.632284 |
| morphogenesis | | | | | |
| (GO:0072171) | | | | | |
| regulation of | 0.598834 | 0.589531 | 0.588808 | 0.576591 | 0.629279 |
| phosphorylation | | | | | |
| (GO:0042325) | | | | | |
| regulation of | 0.613074 | 0.613073 | 0.613899 | 0.648588 | 0.638619 |
| catalytic activity | | | | | |
| (GO:0050790) | | | | | |
| blood vessel | 0.614703 | 0.614686 | 0.615527 | 0.605309 | 0.619781 |
| development | | | | | |
| (GO:0001568) | | | | | |
| positive regulation | 0.614385 | 0.614373 | 0.615216 | 0.608863 | 0.669222 |
| of cell adhesion | | | | | |
| (GO:0045785) | | | | | |
| respiratory system | 0.807582 | 0.708373 | 0.829405 | 0.723765 | 0.72143 |
| development | | | | | |
| (GO:0060541) | | | | | |
| regulation of | 0.613502 | 0.613503 | 0.614318 | 0.557265 | 0.574553 |
| hydrolase activity | | | | | |
| (GO:0051336) | | | | | |
| positive regulation | 0.613158 | 0.613157 | 0.613995 | 0.589983 | 0.626425 |
| of response to | | | | | |
| stimulus | | | | | |
| (GO:0048584) | | | | | |
| ureteric bud | 0.614255 | 0.614239 | 0.615045 | 0.633275 | 0.656987 |
| development | | | | | |
| (GO:0001657) | | | | | |
| regulation of | 0.681259 | 0.67522 | 0.712732 | 0.709163 | 0.743842 |
| primary metabolic | | | | | |
| process | | | | | |
| (GO:0080090) | | | | | |
| regulation of | 0.625473 | 0.632826 | 0.646806 | 0.690805 | 0.696867 |
| extracellular matrix | | | | | |
| organization | | | | | |
| (GO:1903053) | | | | | |
| heart valve | 0.612677 | 0.612677 | 0.613564 | 0.583076 | 0.617287 |
| development | | | | | |
| (GO:0003170) | | | | | |
| neuron | 0.613666 | 0.61366 | 0.614501 | 0.598202 | 0.629292 |
| differentiation | | | | | |
| (GO:0030182) | | | | | |
| negative regulation | 0.571755 | 0.560759 | 0.52589 | 0.619851 | 0.648864 |
| of Wnt signaling | | | | | |
| pathway | | | | | |
| (GO:0030178) | | | | | |
| cell motility | 0.538488 | 0.5142 | 0.510573 | 0.611112 | 0.632177 |
| (GO:0048870) | | | | | |
| regulation of | 0.614474 | 0.61448 | 0.615316 | 0.603484 | 0.620136 |
| endothelial cell | | | | | |
| proliferation | | | | | |
| (GO:0001936) | | | | | |
| cellular response to | 0.616644 | 0.616583 | 0.617435 | 0.554731 | 0.599718 |
| vascular endothelial | | | | | |
| growth factor | | | | | |
| stimulus | | | | | |
| (GO:0035924) | | | | | |
| regulation of BMP | 0.61278 | 0.61278 | 0.613668 | 0.644757 | 0.633 |
| signaling pathway | | | | | |
| (GO:0030510) | | | | | |
| regulation of | 0.612698 | 0.612698 | 0.613585 | 0.706066 | 0.697152 |
| canonical Wnt | | | | | |
| signaling pathway | | | | | |
| (GO:0060828) | | | | | |
| negative regulation | 0.591212 | 0.586468 | 0.562662 | 0.575125 | 0.631422 |
| of cell population | | | | | |
| proliferation | | | | | |
| (GO:0008285) | | | | | |
| lung development | 0.610741 | 0.610893 | 0.611493 | 0.712727 | 0.695308 |
| (GO:0030324) | | | | | |
| positive regulation | 0.614001 | 0.614009 | 0.614848 | 0.610656 | 0.607233 |
| of endothelial cell | | | | | |
| proliferation | | | | | |
| (GO:0001938) | | | | | |
| brain development | 0.602245 | 0.599246 | 0.592716 | 0.646136 | 0.668773 |
| (GO:0007420) | | | | | |
| hemostasis | 0.602523 | 0.600033 | 0.602797 | 0.611058 | 0.662625 |
| (GO:0007599) | | | | | |
| cell-matrix | 0.556355 | 0.542575 | 0.540898 | 0.598139 | 0.596415 |
| adhesion | | | | | |
| (GO:0007160) | | | | | |
| regulation of | 0.612783 | 0.612784 | 0.613633 | 0.581432 | 0.619068 |
| developmental | | | | | |
| process | | | | | |
| (GO:0050793) | | | | | |
| aortic valve | 0.612693 | 0.612693 | 0.61358 | 0.604715 | 0.632208 |
| development | | | | | |
| (GO:0003176) | | | | | |
| regulation of | 0.677848 | 0.669057 | 0.705183 | 0.71183 | 0.738877 |
| nitrogen compound | | | | | |
| metabolic process | | | | | |
| (GO:0051171) | | | | | |
| negative regulation | 0.587216 | 0.584962 | 0.585303 | 0.656177 | 0.699305 |
| of nitrogen | | | | | |
| compound | | | | | |
| metabolic process | | | | | |
| (GO:0051172) | | | | | |
| regulation of | 0.612355 | 0.612355 | 0.613212 | 0.591783 | 0.615377 |
| response to external | | | | | |
| stimulus | | | | | |
| (GO:0032101) | | | | | |
| protein | 0.590189 | 0.572747 | 0.575307 | 0.576056 | 0.619412 |
| phosphorylation | | | | | |
| (GO:0006468) | | | | | |
| regulation of | 0.602458 | 0.600338 | 0.602321 | 0.577814 | 0.608411 |
| protein | | | | | |
| modification | | | | | |
| process | | | | | |
| (GO:0031399) | | | | | |
| regulation of | 0.610232 | 0.61027 | 0.597289 | 0.576235 | 0.626103 |
| protein | | | | | |
| phosphorylation | | | | | |
| (GO:0001932) | | | | | |
| growth | 0.600194 | 0.598628 | 0.600675 | 0.579369 | 0.556979 |
| (GO:0040007) | | | | | |
| developmental | 0.594095 | 0.590889 | 0.593642 | 0.579186 | 0.583893 |
| growth | | | | | |
| (GO:0048589) | | | | | |
| response to BMP | 0.600176 | 0.599954 | 0.601408 | 0.644062 | 0.630834 |
| (GO:0071772) | | | | | |
| regulation of cell | 0.54792 | 0.522448 | 0.525059 | 0.60816 | 0.624918 |
| migration | | | | | |
| (GO:0030334) | | | | | |
| ureteric bud | 0.615285 | 0.615242 | 0.616039 | 0.568275 | 0.600277 |
| morphogenesis | | | | | |
| (GO:0060675) | | | | | |
| glomerulus | 0.613576 | 0.613569 | 0.614447 | 0.638104 | 0.644834 |
| development | | | | | |
| (GO:0032835) | | | | | |
| regulation of | 0.614277 | 0.614262 | 0.615097 | 0.590197 | 0.643196 |
| MAPK cascade | | | | | |
| (GO:0043408) | | | | | |
| regulation of Wnt | 0.614514 | 0.614493 | 0.615316 | 0.674897 | 0.684348 |
| signaling pathway | | | | | |
| (GO:0030111) | | | | | |
| morphogenesis of a | 0.612735 | 0.612735 | 0.613622 | 0.610639 | 0.634375 |
| branching structure | | | | | |
| (GO:0001763) | | | | | |
| cell morphogenesis | 0.613765 | 0.613755 | 0.614587 | 0.576954 | 0.59004 |
| (GO:0000902) | | | | | |
| blood coagulation | 0.612711 | 0.612711 | 0.6136 | 0.615119 | 0.66115 |
| (GO:0007596) | | | | | |
| morphogenesis of a | 0.612735 | 0.612735 | 0.613622 | 0.610639 | 0.634375 |
| branching | | | | | |
| epithelium | | | | | |
| (GO:0061138) | | | | | |
| artery development | 0.612797 | 0.612797 | 0.613684 | 0.618752 | 0.628391 |
| (GO:0060840) | | | | | |
| response to external | 0.629351 | 0.627086 | 0.635241 | 0.621525 | 0.638267 |
| stimulus | | | | | |
| (GO:0009605) | | | | | |
| developmental | 0.618667 | 0.617621 | 0.621139 | 0.642219 | 0.649343 |
| process | | | | | |
| (GO:0032502) | | | | | |
| regulation of | 0.563675 | 0.54227 | 0.543637 | 0.610539 | 0.631563 |
| locomotion | | | | | |
| (GO:0040012) | | | | | |
| BMP signaling | 0.612636 | 0.612636 | 0.604974 | 0.628423 | 0.632865 |
| pathway | | | | | |
| (GO:0030509) | | | | | |
| negative regulation | 0.612726 | 0.612726 | 0.613613 | 0.649436 | 0.690414 |
| of canonical Wnt | | | | | |
| signaling pathway | | | | | |
| (GO:0090090) | | | | | |
| mesenchyme | 0.612727 | 0.612728 | 0.613617 | 0.623272 | 0.601875 |
| development | | | | | |
| (GO:0060485) | | | | | |
| heart valve | 0.56702 | 0.559876 | 0.527849 | 0.577261 | 0.620695 |
| morphogenesis | | | | | |
| (GO:0003179) | | | | | |
| cellular response to | 0.613174 | 0.613175 | 0.614018 | 0.605361 | 0.626711 |
| oxygen-containing | | | | | |
| compound | | | | | |
| (GO:1901701) | | | | | |
| aortic valve | 0.61274 | 0.61274 | 0.613626 | 0.5959 | 0.645624 |
| morphogenesis | | | | | |
| (GO:0003180) | | | | | |
| negative regulation | 0.638759 | 0.639573 | 0.651319 | 0.65929 | 0.684848 |
| of DNA-templated | | | | | |
| transcription | | | | | |
| (GO:0045892) | | | | | |
| branching involved | 0.612687 | 0.612687 | 0.613573 | 0.564228 | 0.603052 |
| in ureteric bud | | | | | |
| morphogenesis | | | | | |
| (GO:0001658) | | | | | |
| regulation of | 0.614143 | 0.614137 | 0.61496 | 0.59998 | 0.62372 |
| protein metabolic | | | | | |
| process | | | | | |
| (GO:0051246) | | | | | |
| regulation of | 0.599429 | 0.593973 | 0.597799 | 0.716716 | 0.729101 |
| collagen metabolic | | | | | |
| process | | | | | |
| (GO:0010712) | | | | | |
| tissue development | 0.51524 | 0.532932 | 0.495089 | 0.584839 | 0.625564 |
| (GO:0009888) | | | | | |
| positive regulation of | 0.605281 | 0.603227 | 0.606003 | 0.606512 | 0.636377 |
| phosphatidylinositol | | | | | |
| 3-kinase/protein | | | | | |
| kinase B signal | | | | | |
| transduction | | | | | |
| (GO:0051897) | | | | | |
| cellular anatomical | 0.612794 | 0.612795 | 0.613643 | 0.579059 | 0.589172 |
| entity | | | | | |
| morphogenesis | | | | | |
| (GO:0032989) | | | | | |
| neuron projection | 0.614714 | 0.614692 | 0.615528 | 0.560275 | 0.614186 |
| development | | | | | |
| (GO:0031175) | | | | | |
| cellular response to | 0.612725 | 0.612725 | 0.613614 | 0.624346 | 0.611801 |
| transforming | | | | | |
| growth factor beta | | | | | |
| stimulus | | | | | |
| (GO:0071560) | | | | | |
| regulation of actin | 0.594688 | 0.582136 | 0.584693 | 0.624342 | 0.645616 |
| filament-based | | | | | |
| process | | | | | |
| (GO:0032970) | | | | | |
| sprouting | 0.612275 | 0.612293 | 0.613137 | 0.703223 | 0.68836 |
| angiogenesis | | | | | |
| (GO:0002040) | | | | | |
| response to stress | 0.614612 | 0.614053 | 0.615586 | 0.584344 | 0.613169 |
| (GO:0006950) | | | | | |
| MAPK cascade | 0.600589 | 0.59976 | 0.601505 | 0.593036 | 0.642076 |
| (GO:0000165) | | | | | |
| regulation of wound | 0.61263 | 0.612631 | 0.613519 | 0.649479 | 0.66724 |
| healing | | | | | |
| (GO:0061041) | | | | | |
| renal system | 0.615716 | 0.615664 | 0.61652 | 0.647901 | 0.64459 |
| vasculature | | | | | |
| development | | | | | |
| (GO:0061437) | | | | | |
| blood vessel | 0.593888 | 0.588807 | 0.591872 | 0.570207 | 0.613084 |
| morphogenesis | | | | | |
| (GO:0048514) | | | | | |
| positive regulation | 0.610978 | 0.610995 | 0.611864 | 0.701615 | 0.686442 |
| of cellular | | | | | |
| component | | | | | |
| biogenesis | | | | | |
| (GO:0044089) | | | | | |
| positive regulation | 0.586639 | 0.581454 | 0.551158 | 0.521451 | 0.614245 |
| of apoptotic process | | | | | |
| (GO:0043065) | | | | | |
| vasculogenesis | 0.615259 | 0.615239 | 0.616079 | 0.661525 | 0.596623 |
| (GO:0001570) | | | | | |
| collagen | 0.612769 | 0.612769 | 0.613656 | 0.57282 | 0.617075 |
| biosynthetic process | | | | | |
| (GO:0032964) | | | | | |
| forebrain | 0.612644 | 0.612644 | 0.613532 | 0.719886 | 0.682776 |
| development | | | | | |
| (GO:0030900) | | | | | |
| response to | 0.597851 | 0.598786 | 0.585495 | 0.625059 | 0.684563 |
| hormone | | | | | |
| (GO:0009725) | | | | | |
| response to | 0.664844 | 0.668339 | 0.694313 | 0.674713 | 0.638372 |
| cytokine | | | | | |
| (GO:0034097) | | | | | |
| ear development | 0.612614 | 0.612615 | 0.613502 | 0.638656 | 0.695652 |
| (GO:0043583) | | | | | |
| endothelial cell | 0.536565 | 0.538363 | 0.539125 | 0.686613 | 0.697893 |
| apoptotic process | | | | | |
| (GO:0072577) | | | | | |
| phosphorylation | 0.59235 | 0.580333 | 0.579035 | 0.568159 | 0.618094 |
| (GO:0016310) | | | | | |
| response to | 0.562389 | 0.574001 | 0.553535 | 0.59115 | 0.641285 |
| endogenous | | | | | |
| stimulus | | | | | |
| (GO:0009719) | | | | | |
| regulation of | 0.612734 | 0.612734 | 0.613622 | 0.595189 | 0.608595 |
| hemostasis | | | | | |
| (GO:1900046) | | | | | |
| artery | 0.612782 | 0.612782 | 0.613669 | 0.625318 | 0.648128 |
| morphogenesis | | | | | |
| (GO:0048844) | | | | | |
| regulation of body | 0.604492 | 0.601912 | 0.604503 | 0.609792 | 0.665995 |
| fluid levels | | | | | |
| (GO:0050878) | | | | | |
| regulation of cell | 0.612587 | 0.612586 | 0.613433 | 0.531374 | 0.574301 |
| development | | | | | |
| (GO:0060284) | | | | | |
| negative regulation | 0.627402 | 0.642725 | 0.671802 | 0.569055 | 0.744453 |
| of programmed cell | | | | | |
| death | | | | | |
| (GO:0043069) | | | | | |
| positive regulation | 0.619638 | 0.621357 | 0.623637 | 0.65595 | 0.652433 |
| of cellular | | | | | |
| component | | | | | |
| organization | | | | | |
| (GO:0051130) | | | | | |
| negative regulation | 0.653592 | 0.708876 | 0.751337 | 0.764229 | 0.669898 |
| of apoptotic process | | | | | |
| (GO:0043066) | | | | | |
| endoderm | 0.524154 | 0.494466 | 0.438878 | 0.537847 | 0.635924 |
| development | | | | | |
| (GO:0007492) | | | | | |
| negative regulation | 0.722597 | 0.720485 | 0.770244 | 0.69155 | 0.769281 |
| of BMP signaling | | | | | |
| pathway | | | | | |
| (GO:0030514) | | | | | |
| branching | 0.612736 | 0.612737 | 0.613623 | 0.61331 | 0.616171 |
| morphogenesis of | | | | | |
| an epithelial tube | | | | | |
| (GO:0048754) | | | | | |
| biological | 0.669541 | 0.666334 | 0.704409 | 0.694992 | 0.70782 |
| regulation | | | | | |
| (GO:0065007) | | | | | |
| positive regulation | 0.613593 | 0.613583 | 0.614424 | 0.607677 | 0.634321 |
| of signal | | | | | |
| transduction | | | | | |
| (GO:0009967) | | | | | |
| endodenn. | 0.56447 | 0.557501 | 0.539913 | 0.584544 | 0.605606 |
| formation | | | | | |
| (GO:0001706) | | | | | |
| mesenchyme | 0.620538 | 0.617693 | 0.622329 | 0.788787 | 0.763785 |
| morphogenesis | | | | | |
| (GO:0072132) | | | | | |
| positive regulation | 0.613229 | 0.613229 | 0.614047 | 0.549463 | 0.571676 |
| of catalytic activity | | | | | |
| (GO:0043085) | | | | | |
| cell-cell adhesion | 0.613795 | 0.613789 | 0.614632 | 0.647022 | 0.651693 |
| (GO:0098609) | | | | | |
| stem cell | 0.61279 | 0.61279 | 0.613678 | 0.624552 | 0.627555 |
| proliferation | | | | | |
| (GO:0072089) | | | | | |
| endothelial cell | 0.6153 | 0.615264 | 0.616088 | 0.581079 | 0.711978 |
| migration | | | | | |
| (GO:0043542) | | | | | |
| positive regulation | 0.612166 | 0.612164 | 0.612997 | 0.570725 | 0.555919 |
| of hydrolase | | | | | |
| activity | | | | | |
| (GO:0051345) | | | | | |
| positive regulation | 0.584011 | 0.582999 | 0.582483 | 0.654366 | 0.647893 |
| of molecular | | | | | |
| function | | | | | |
| (GO:0044093) | | | | | |
| response to abiotic | 0.583141 | 0.588244 | 0.55489 | 0.556663 | 0.580497 |
| stimulus | | | | | |
| (GO:0009628) | | | | | |
| ossification | 0.614666 | 0.614633 | 0.615473 | 0.587773 | 0.605154 |
| (GO:0001503) | | | | | |
| response to | 0.613763 | 0.613766 | 0.614594 | 0.626917 | 0.621836 |
| inorganic substance | | | | | |
| (GO:0010035) | | | | | |
| sensory organ | 0.612887 | 0.612887 | 0.613776 | 0.645341 | 0.635901 |
| morphogenesis | | | | | |
| (GO:0090596) | | | | | |
| heart | 0.614171 | 0.614154 | 0.614998 | 0.567599 | 0.620735 |
| morphogenesis | | | | | |
| (GO:0003007) | | | | | |
| regulation of stem | 0.612793 | 0.612793 | 0.613679 | 0.593646 | 0.601379 |
| cell proliferation | | | | | |
| (GO:0072091) | | | | | |
| regulation of | 0.602978 | 0.60203 | 0.60372 | 0.640897 | 0.606791 |
| chemotaxis | | | | | |
| (GO:0050920) | | | | | |
| organonitrogen | 0.647862 | 0.650935 | 0.668062 | 0.634651 | 0.698504 |
| compound | | | | | |
| metabolic process | | | | | |
| (GO:1901564) | | | | | |
| positive regulation | 0.616727 | 0.616727 | 0.61755 | 0.651783 | 0.626197 |
| of chemotaxis | | | | | |
| (GO:0050921) | | | | | |
| bone mineralization | 0.584957 | 0.577512 | 0.577291 | 0.624768 | 0.630758 |
| (GO:0030282) | | | | | |
| developmental | 0.612726 | 0.612727 | 0.613615 | 0.655402 | 0.618417 |
| growth involved in | | | | | |
| morphogenesis | | | | | |
| (GO:0060560) | | | | | |
| mesenchymal cell | 0.612667 | 0.612667 | 0.613556 | 0.666102 | 0.632218 |
| differentiation | | | | | |
| (GO:0048762) | | | | | |
| regulation of cell | 0.595047 | 0.591404 | 0.594649 | 0.611201 | 0.625772 |
| motility | | | | | |
| (GO:2000145) | | | | | |
| response to organic | 0.59009 | 0.600879 | 0.592355 | 0.606381 | 0.640293 |
| cyclic compound | | | | | |
| (GO:0014070) | | | | | |
| chemotaxis | 0.605785 | 0.605766 | 0.607242 | 0.591288 | 0.615607 |
| (GO:0006935) | | | | | |
| phosphatidylinositol | 0.612717 | 0.612718 | 0.613606 | 0.624322 | 0.677578 |
| 3-kinase/protein | | | | | |
| kinase B signal | | | | | |
| transduction | | | | | |
| (GO:0043491) | | | | | |
| regulation of | 0.611305 | 0.611315 | 0.612174 | 0.620316 | 0.646745 |
| cytoskeleton | | | | | |
| organization | | | | | |
| (GO:0051493) | | | | | |
| negative regulation | 0.612551 | 0.612553 | 0.613392 | 0.69149 | 0.749646 |
| of nucleobase- | | | | | |
| containing | | | | | |
| compound | | | | | |
| metabolic process | | | | | |
| (GO:0045934) | | | | | |
| regulation of | 0.611447 | 0.611456 | 0.612325 | 0.695093 | 0.65112 |
| localization | | | | | |
| (GO:0032879) | | | | | |
| regulation of | 0.541048 | 0.499944 | 0.497387 | 0.550322 | 0.615822 |
| epithelial cell | | | | | |
| migration | | | | | |
| (GO:0010632) | | | | | |
| negative regulation | 0.627497 | 0.628346 | 0.635161 | 0.661622 | 0.685885 |
| of RNA metabolic | | | | | |
| process | | | | | |
| (GO:0051253) | | | | | |
| gland | 0.61276 | 0.61276 | 0.613647 | 0.60011 | 0.649166 |
| morphogenesis | | | | | |
| (GO:0022612) | | | | | |
| endodermal cell | 0.56447 | 0.557501 | 0.539913 | 0.584544 | 0.605606 |
| differentiation | | | | | |
| (GO:0035987) | | | | | |
| negative regulation | 0.629852 | 0.629939 | 0.63951 | 0.654832 | 0.650548 |
| of biological | | | | | |
| process | | | | | |
| (GO:0048519) | | | | | |
| positive regulation | 0.61366 | 0.613651 | 0.614484 | 0.597702 | 0.645114 |
| of intracellular | | | | | |
| signal transduction | | | | | |
| (GO:1902533) | | | | | |
| positive regulation | 0.61399 | 0.61398 | 0.614808 | 0.570345 | 0.619907 |
| of phosphorylation | | | | | |
| (GO:0042327) | | | | | |
| muscle tissue | 0.612764 | 0.612763 | 0.613653 | 0.602444 | 0.610851 |
| development | | | | | |
| (GO:0060537) | | | | | |
| pericyte cell | 0.613216 | 0.613457 | 0.61405 | 0.689027 | 0.695374 |
| differentiation | | | | | |
| (GO:1904238) | | | | | |
| skeletal system | 0.612817 | 0.612817 | 0.613704 | 0.627294 | 0.616951 |
| morphogenesis | | | | | |
| (GO:0048705) | | | | | |
| endocardial cushion | 0.612715 | 0.612715 | 0.613601 | 0.525034 | 0.596292 |
| formation | | | | | |
| (GO:0003272) | | | | | |
| negative regulation | 0.601585 | 0.601531 | 0.603053 | 0.574358 | 0.583569 |
| of cell development | | | | | |
| (GO:0010721) | | | | | |
| regulation of | 0.547484 | 0.548848 | 0.550039 | 0.595756 | 0.635587 |
| endothelial cell | | | | | |
| apoptotic process | | | | | |
| (GO:2000351) | | | | | |
| endocardial cushion | 0.612708 | 0.612708 | 0.613594 | 0.573334 | 0.624 |
| development | | | | | |
| (GO:0003197) | | | | | |
| positive regulation | 0.614244 | 0.614252 | 0.615085 | 0.602948 | 0.644892 |
| of response to | | | | | |
| external stimulus | | | | | |
| (GO:0032103) | | | | | |
| enzyme-linked | 0.613997 | 0.613988 | 0.614828 | 0.598817 | 0.638346 |
| receptor protein | | | | | |
| signaling pathway | | | | | |
| (GO:0007167) | | | | | |
| positive regulation | 0.628733 | 0.628603 | 0.63868 | 0.679715 | 0.702744 |
| of cellular | | | | | |
| metabolic process | | | | | |
| (GO:0031325) | | | | | |
| neuron | 0.61417 | 0.614158 | 0.614998 | 0.587414 | 0.612584 |
| development | | | | | |
| (GO:0048666) | | | | | |
| positive regulation | 0.601187 | 0.598606 | 0.600832 | 0.596545 | 0.618347 |
| of cell | | | | | |
| communication | | | | | |
| (GO:0010647) | | | | | |
| regulation of cell | 0.614274 | 0.614262 | 0.615087 | 0.573463 | 0.598863 |
| projection | | | | | |
| organization | | | | | |
| (GO:0031344) | | | | | |
| cellular response to | 0.704992 | 0.627282 | 0.860746 | 0.933498 | 0.786383 |
| cytokine stimulus | | | | | |
| (GO:0071345) | | | | | |
| eye development | 0.56711 | 0.559406 | 0.517175 | 0.642841 | 0.636352 |
| (GO:0001654) | | | | | |
| glomerulus | 0.63384 | 0.640033 | 0.639032 | 0.482044 | 0.652499 |
| vasculature | | | | | |
| development | | | | | |
| (GO:0072012) | | | | | |
| regulation of | 0.612671 | 0.612672 | 0.61356 | 0.681383 | 0.655182 |
| epithelial to | | | | | |
| mesenchymal | | | | | |
| transition | | | | | |
| (GO:0010717) | | | | | |
| response to | 0.613788 | 0.613793 | 0.614642 | 0.672367 | 0.648295 |
| organonitrogen | | | | | |
| compound | | | | | |
| (GO:0010243) | | | | | |
| metanephric | 0.612736 | 0.612736 | 0.613623 | 0.644555 | 0.637987 |
| nephron | | | | | |
| development | | | | | |
| (GO:0072210) | | | | | |
| cellular response to | 0.612835 | 0.612835 | 0.613728 | 0.562877 | 0.607039 |
| growth factor | | | | | |
| stimulus | | | | | |
| (GO:0071363) | | | | | |
| endocardial cushion | 0.612724 | 0.612724 | 0.61361 | 0.538488 | 0.589269 |
| morphogenesis | | | | | |
| (GO:0003203) | | | | | |
| positive regulation | 0.61274 | 0.61274 | 0.613627 | 0.664409 | 0.645076 |
| of transmembrane | | | | | |
| receptor protein | | | | | |
| serine/threonine | | | | | |
| kinase signaling | | | | | |
| pathway | | | | | |
| (GO:0090100) | | | | | |
| regulation of | 0.614048 | 0.614016 | 0.614865 | 0.531788 | 0.635023 |
| biomineral tissue | | | | | |
| development | | | | | |
| (GO:0070167) | | | | | |
| regulation of | 0.676369 | 0.669115 | 0.699401 | 0.692439 | 0.73778 |
| metabolic process | | | | | |
| (GO:0019222) | | | | | |
| tendon development | 0.618446 | 0.618289 | 0.619202 | 0.660271 | 0.688568 |
| (GO:0035989) | | | | | |
| regulation of | 0.731174 | 0.726543 | 0.760845 | 0.722064 | 0.737386 |
| supramolecular | | | | | |
| fiber organization | | | | | |
| (GO:1902903) | | | | | |
| regulation of blood | 0.612733 | 0.612733 | 0.61362 | 0.599338 | 0.617267 |
| coagulation | | | | | |
| (GO:0030193) | | | | | |
| response to nitrogen | 0.61327 | 0.613272 | 0.614131 | 0.684931 | 0.65229 |
| compound | | | | | |
| (GO:1901698) | | | | | |
| regulation of | 0.611224 | 0.611231 | 0.612074 | 0.705931 | 0.705872 |
| transferase activity | | | | | |
| (GO:0051338) | | | | | |
| positive regulation | 0.612644 | 0.612645 | 0.613534 | 0.651307 | 0.539481 |
| of MAPK cascade | | | | | |
| (GO:0043410) | | | | | |
| system | 0.581397 | 0.585344 | 0.572855 | 0.63156 | 0.645621 |
| development | | | | | |
| (GO:0048731) | | | | | |
| response to growth | 0.569246 | 0.563672 | 0.525617 | 0.552749 | 0.597136 |
| factor | | | | | |
| (GO:0070848) | | | | | |
| positive regulation | 0.612711 | 0.612711 | 0.613598 | 0.709153 | 0.604839 |
| of epithelial to | | | | | |
| mesenchymal | | | | | |
| transition | | | | | |
| (GO:0010718) | | | | | |
| regulation of | 0.614274 | 0.614262 | 0.615087 | 0.573463 | 0.598863 |
| plasma membrane | | | | | |
| bounded cell | | | | | |
| projection | | | | | |
| organization | | | | | |
| (GO:0120035) | | | | | |
| regulation of cell- | 0.613261 | 0.613258 | 0.61413 | 0.606766 | 0.5965 |
| matrix adhesion | | | | | |
| (GO:0001952) | | | | | |
| positive regulation | 0.614644 | 0.614628 | 0.615446 | 0.565759 | 0.613433 |
| of protein metabolic | | | | | |
| process | | | | | |
| (GO:0051247) | | | | | |
| bone | 0.612756 | 0.612756 | 0.613642 | 0.636846 | 0.635532 |
| morphogenesis | | | | | |
| (GO:0060349) | | | | | |
| negative regulation | 0.640262 | 0.636577 | 0.644363 | 0.644517 | 0.677732 |
| of cartilage | | | | | |
| development | | | | | |
| (GO:0061037) | | | | | |
| response to lipid | 0.612998 | 0.612999 | 0.613858 | 0.69973 | 0.718533 |
| (GO:0033993) | | | | | |
| regulation of | 0.615537 | 0.615479 | 0.616334 | 0.606512 | 0.636377 |
| phosphatidylinositol | | | | | |
| 3-kinase/protein | | | | | |
| kinase B signal | | | | | |
| transduction | | | | | |
| (GO:0051896) | | | | | |
| positive regulation | 0.61443 | 0.614412 | 0.615241 | 0.635091 | 0.665194 |
| of protein | | | | | |
| phosphorylation | | | | | |
| (GO:0001934) | | | | | |
| regulation of | 0.668506 | 0.663162 | 0.689332 | 0.692133 | 0.714343 |
| macromolecule | | | | | |
| metabolic process | | | | | |
| (GO:0060255) | | | | | |
| negative regulation | 0.624696 | 0.625887 | 0.632654 | 0.66199 | 0.657314 |
| of cellular process | | | | | |
| (GO:0048523) | | | | | |
| regulation of | 0.615145 | 0.615045 | 0.615906 | 0.648636 | 0.624522 |
| endothelial cell | | | | | |
| chemotaxis | | | | | |
| (GO:2001026) | | | | | |
| positive chemotaxis | 0.623747 | 0.623362 | 0.625168 | 0.631728 | 0.620806 |
| (GO:0050918) | | | | | |
| negative regulation | 0.611586 | 0.611601 | 0.612443 | 0.6708 | 0.66696 |
| of cellular | | | | | |
| component | | | | | |
| organization | | | | | |
| (GO:0051129) | | | | | |
| cell chemotaxis | 0.615702 | 0.615702 | 0.616539 | 0.611054 | 0.632523 |
| (GO:0060326) | | | | | |
| response to oxygen | 0.612743 | 0.612743 | 0.61363 | 0.674497 | 0.729627 |
| levels | | | | | |
| (GO:0070482) | | | | | |
| cardiac chamber | 0.568143 | 0.575808 | 0.545972 | 0.56491 | 0.824259 |
| morphogenesis | | | | | |
| (GO:0003206) | | | | | |
| axon development | 0.572559 | 0.569811 | 0.571246 | 0.611593 | 0.586439 |
| (GO:0061564) | | | | | |
| inner ear | 0.612656 | 0.612656 | 0.613544 | 0.686215 | 0.612222 |
| development | | | | | |
| (GO:0048839) | | | | | |
| organ growth | 0.612851 | 0.612851 | 0.613738 | 0.589547 | 0.611336 |
| (GO:0035265) | | | | | |
| cell junction | 0.612858 | 0.612859 | 0.613692 | 0.610847 | 0.661 |
| assembly | | | | | |
| (GO:0034329) | | | | | |
| locomotion | 0.566461 | 0.553522 | 0.547717 | 0.605351 | 0.630887 |
| (GO:0040011) | | | | | |
| vascular endothelial | 0.612726 | 0.612726 | 0.613613 | 0.386635 | 0.645275 |
| growth factor | | | | | |
| signaling pathway | | | | | |
| (GO:0038084) | | | | | |
| positive regulation | 0.611582 | 0.611549 | 0.612168 | 0.656699 | 0.672587 |
| of macromolecule | | | | | |
| metabolic process | | | | | |
| (GO:0010604) | | | | | |
| negative regulation | 0.612883 | 0.612884 | 0.613718 | 0.616684 | 0.602907 |
| of cell-substrate | | | | | |
| adhesion | | | | | |
| (GO:0010812) | | | | | |
| epithelial to | 0.612605 | 0.612605 | 0.613494 | 0.69335 | 0.672661 |
| mesenchymal | | | | | |
| transition | | | | | |
| (GO:0001837) | | | | | |
| cardiac ventricle | 0.631587 | 0.636266 | 0.640203 | 0.682604 | 0.727937 |
| development | | | | | |
| (GO:0003231) | | | | | |
| positive regulation | 0.600012 | 0.59829 | 0.600239 | 0.63534 | 0.63639 |
| of protein | | | | | |
| modification | | | | | |
| process | | | | | |
| (GO:0031401) | | | | | |
| negative regulation | 0.57281 | 0.57344 | 0.55317 | 0.705363 | 0.57871 |
| of transmembrane | | | | | |
| receptor protein | | | | | |
| serine/threonine | | | | | |
| kinase signaling | | | | | |
| pathway | | | | | |
| (GO:0090101) | | | | | |
| post-embryonic eye | 0.712594 | 0.687702 | 0.742104 | 0.819678 | 0.807412 |
| morphogenesis | | | | | |
| (GO:0048050) | | | | | |
| formation of | 0.612851 | 0.612851 | 0.613739 | 0.58145 | 0.643206 |
| primary germ layer | | | | | |
| (GO:0001704) | | | | | |
| roof of mouth | 0.612718 | 0.612718 | 0.613605 | 0.694049 | 0.694964 |
| development | | | | | |
| (GO:0060021) | | | | | |
| anatomical structure | 0.562705 | 0.561398 | 0.512735 | 0.594743 | 0.633924 |
| formation involved | | | | | |
| in morphogenesis | | | | | |
| (GO:0048646) | | | | | |
| extracellular matrix | 0.612746 | 0.612746 | 0.613634 | 0.61751 | 0.616411 |
| assembly | | | | | |
| (GO:0085029) | | | | | |
| protein | 0.610231 | 0.60869 | 0.608995 | 0.601848 | 0.664447 |
| modification | | | | | |
| process | | | | | |
| (GO:0036211) | | | | | |
| gland development | 0.612693 | 0.612693 | 0.613581 | 0.594763 | 0.651676 |
| (GO:0048732) | | | | | |
| muscle cell | 0.612739 | 0.612739 | 0.613627 | 0.59574 | 0.641521 |
| development | | | | | |
| (GO:0055001) | | | | | |
| cellular component | 0.675277 | 0.67802 | 0.697712 | 0.72743 | 0.725791 |
| organization | | | | | |
| (GO:0016043) | | | | | |
| positive regulation | 0.589184 | 0.58587 | 0.587757 | 0.571335 | 0.619094 |
| of phosphorus | | | | | |
| metabolic process | | | | | |
| (GO:0010562) | | | | | |
| regulation of | 0.614229 | 0.614176 | 0.615034 | 0.574266 | 0.641693 |
| collagen | | | | | |
| biosynthetic process | | | | | |
| (GO:0032965) | | | | | |
| negative regulation | 0.616571 | 0.616537 | 0.617373 | 0.655442 | 0.680677 |
| of smooth muscle | | | | | |
| cell migration | | | | | |
| (GO:0014912) | | | | | |
| extracellular matrix | 0.612802 | 0.612802 | 0.613688 | 0.609047 | 0.676783 |
| disassembly | | | | | |
| (GO:0022617) | | | | | |
| regulation of | 0.612762 | 0.612762 | 0.613648 | 0.588405 | 0.603806 |
| extracellular matrix | | | | | |
| disassembly | | | | | |
| (GO:0010715) | | | | | |
| animal organ | 0.614785 | 0.614766 | 0.615606 | 0.544429 | 0.593694 |
| morphogenesis | | | | | |
| (GO:0009887) | | | | | |
| mesenchymal cell | 0.596633 | 0.596413 | 0.596088 | 0.623968 | 0.653027 |
| proliferation | | | | | |
| (GO:0010463) | | | | | |
| endochondral bone | 0.612754 | 0.612754 | 0.613641 | 0.649106 | 0.677049 |
| morphogenesis | | | | | |
| (GO:0060350) | | | | | |
| positive regulation | 0.618944 | 0.618846 | 0.623265 | 0.662645 | 0.690341 |
| of metabolic | | | | | |
| process | | | | | |
| (GO:0009893) | | | | | |
| inflammatory | 0.611661 | 0.611653 | 0.612533 | 0.534076 | 0.584863 |
| response | | | | | |
| (GO:0006954) | | | | | |
| gastrulation | 0.585143 | 0.563291 | 0.562583 | 0.642635 | 0.674725 |
| (GO:0007369) | | | | | |
| regulation of actin | 0.609352 | 0.605278 | 0.607008 | 0.634522 | 0.642369 |
| cytoskeleton | | | | | |
| organization | | | | | |
| (GO:0032956) | | | | | |
| blood vessel | 0.640813 | 0.657106 | 0.691046 | 0.75122 | 0.736029 |
| endothelial cell | | | | | |
| proliferation | | | | | |
| involved in | | | | | |
| sprouting | | | | | |
| angiogenesis | | | | | |
| (GO:0002043) | | | | | |
| neuronal stem cell | 0.577189 | 0.578135 | 0.57029 | 0.641876 | 0.651175 |
| population | | | | | |
| maintenance | | | | | |
| (GO:0097150) | | | | | |
| regulation of | 0.690648 | 0.680726 | 0.724848 | 0.709797 | 0.746075 |
| cellular metabolic | | | | | |
| process | | | | | |
| (GO:0031323) | | | | | |
| reproductive | 0.613664 | 0.613657 | 0.614536 | 0.628511 | 0.749952 |
| structure | | | | | |
| development | | | | | |
| (GO:0048608) | | | | | |
| regulation of | 0.673771 | 0.671293 | 0.708436 | 0.696196 | 0.713721 |
| biological process | | | | | |
| (GO:0050789) | | | | | |
| phosphorus | 0.589431 | 0.585217 | 0.586848 | 0.603781 | 0.645065 |
| metabolic process | | | | | |
| (GO:0006793) | | | | | |
| axonogenesis | 0.610866 | 0.610879 | 0.570645 | 0.603532 | 0.581672 |
| (GO:0007409) | | | | | |
| digestive tract | 0.561379 | 0.528949 | 0.529245 | 0.66582 | 0.66576 |
| morphogenesis | | | | | |
| (GO:0048546) | | | | | |
| regulation of bone | 0.586243 | 0.57727 | 0.574443 | 0.647528 | 0.678413 |
| mineralization | | | | | |
| (GO:0030500) | | | | | |
| epithelial tube | 0.603733 | 0.603044 | 0.603966 | 0.642723 | 0.66294 |
| morphogenesis | | | | | |
| (GO:0060562) | | | | | |
| regulation of | 0.612425 | 0.612425 | 0.613281 | 0.580901 | 0.612035 |
| response to stress | | | | | |
| (GO:0080134) | | | | | |
| phosphate- | 0.612941 | 0.612941 | 0.613787 | 0.601792 | 0.646566 |
| containing | | | | | |
| compound | | | | | |
| metabolic process | | | | | |
| (GO:0006796) | | | | | |
| regulation of ERK1 | 0.613682 | 0.613671 | 0.614514 | 0.584697 | 0.617032 |
| and ERK2 cascade | | | | | |
| (GO:0070372) | | | | | |
| ovulation cycle | 0.611521 | 0.610967 | 0.612562 | 0.58608 | 0.665541 |
| process | | | | | |
| (GO:0022602) | | | | | |
| renal system | 0.622772 | 0.62295 | 0.624053 | 0.66042 | 0.676946 |
| vasculature | | | | | |
| morphogenesis | | | | | |
| (GO:0061438) | | | | | |
| positive regulation | 0.615304 | 0.615288 | 0.616116 | 0.588385 | 0.607248 |
| of cell migration | | | | | |
| (GO:0030335) | | | | | |
| positive regulation | 0.612713 | 0.612712 | 0.613598 | 0.647008 | 0.670957 |
| of neuroepithelial | | | | | |
| cell differentiation | | | | | |
| (GO:1902913) | | | | | |
| membranous | 0.612721 | 0.612721 | 0.613607 | 0.657561 | 0.676345 |
| septum | | | | | |
| morphogenesis | | | | | |
| (GO:0003149) | | | | | |
| proteolysis | 0.674994 | 0.676963 | 0.765112 | 0.762134 | 0.721129 |
| (GO:0006508) | | | | | |
| regulation of | 0.612785 | 0.612785 | 0.613672 | 0.548915 | 0.600832 |
| smooth muscle cell | | | | | |
| migration | | | | | |
| (GO:0014910) | | | | | |
| cellular response to | 0.671946 | 0.657257 | 0.671684 | 0.787828 | 0.728032 |
| external stimulus | | | | | |
| (GO:0071496) | | | | | |
| organic eyclic | 0.607347 | 0.607421 | 0.608175 | 0.630768 | 0.648367 |
| compound | | | | | |
| biosynthetic process | | | | | |
| (GO:1901362) | | | | | |
| heterocycle | 0.610268 | 0.610275 | 0.611125 | 0.630976 | 0.648401 |
| biosynthetic process | | | | | |
| (GO:0018130) | | | | | |
| aromatic compound | 0.607577 | 0.607645 | 0.608407 | 0.631038 | 0.648512 |
| biosynthetic process | | | | | |
| (GO:0019438) | | | | | |
| nucleobase- | 0.610369 | 0.610375 | 0.611227 | 0.630816 | 0.648419 |
| containing | | | | | |
| compound | | | | | |
| biosynthetic process | | | | | |
| (GO:0034654) | | | | | |
| organonitrogen | 0.608907 | 0.608924 | 0.609749 | 0.630924 | 0.649008 |
| compound | | | | | |
| metabolic process | | | | | |
| (GO:1901564) | | | | | |
| positive regulation | 0.615694 | 0.615681 | 0.616501 | 0.588559 | 0.608349 |
| of locomotion | | | | | |
| (GO:0040017) | | | | | |
| cellular nitrogen | 0.60749 | 0.607559 | 0.608321 | 0.631064 | 0.648415 |
| compound | | | | | |
| biosynthetic process | | | | | |
| (GO:0044271) | | | | | |
| mitotic cell cycle | 0.684863 | 0.738514 | 0.629646 | 0.634066 | 0.655562 |
| (GO:0000278) | | | | | |
| cell cycle process | 0.621239 | 0.628032 | 0.616083 | 0.626395 | 0.644765 |
| (GO:0022402) | | | | | |
| chromosome | 0.605255 | 0.594434 | 0.602202 | 0.671132 | 0.696822 |
| segregation | | | | | |
| (GO:0007059) | | | | | |
| mitotic cell cycle | 0.588718 | 0.576953 | 0.596509 | 0.638229 | 0.655604 |
| process | | | | | |
| (GO:1903047) | | | | | |
| cell cycle | 0.670595 | 0.713431 | 0.625905 | 0.63051 | 0.648653 |
| (GO:0007049) | | | | | |
| mitotic sister | 0.645841 | 0.672073 | 0.647476 | 0.714175 | 0.714166 |
| chromatid | | | | | |
| segregation | | | | | |
| (GO:0000070) | | | | | |
| positive regulation | 0.666078 | 0.67814 | 0.666292 | 0.657859 | 0.654955 |
| of chromosome | | | | | |
| separation | | | | | |
| (GO:1905820) | | | | | |
| regulation of RNA | 0.607572 | 0.607631 | 0.608401 | 0.631752 | 0.648786 |
| metabolic process | | | | | |
| (GO:0051252) | | | | | |
| cell division | 0.678114 | 0.827807 | 0.736351 | 0.71336 | 0.773658 |
| (GO:0051301) | | | | | |
| cellular response to | 0.614859 | 0.614846 | 0.615672 | 0.632989 | 0.654064 |
| endogenous | | | | | |
| stimulus | | | | | |
| (GO:0071495) | | | | | |
| cell cycle | 0.612595 | 0.612595 | 0.613479 | 0.621599 | 0.646079 |
| checkpoint | | | | | |
| signaling | | | | | |
| (GO:0000075) | | | | | |
| developmental | 0.577105 | 0.574238 | 0.578395 | 0.56829 | 0.603882 |
| process | | | | | |
| (GO:0032502) | | | | | |
| macromolecule | 0.681238 | 0.652687 | 0.617023 | 0.766869 | 0.663535 |
| localization | | | | | |
| (GO:0033036) | | | | | |
| anatomical structure | 0.844026 | 0.677741 | 0.616926 | 0.567593 | 0.694353 |
| development | | | | | |
| (GO:0048856) | | | | | |
| biological | 0.593321 | 0.590816 | 0.593794 | 0.62518 | 0.674369 |
| regulation | | | | | |
| (GO:0065007) | | | | | |
| cellular localization | 0.600212 | 0.597999 | 0.600804 | 0.60386 | 0.636144 |
| (GO:0051641) | | | | | |
| protein localization | 0.654276 | 0.664438 | 0.625566 | 0.615741 | 0.61517 |
| (GO:0008104) | | | | | |
| transport | 0.641599 | 0.641663 | 0.642579 | 0.599871 | 0.620525 |
| (GO:0006810) | | | | | |
| regulation of | 0.598877 | 0.597158 | 0.599593 | 0.65124 | 0.685837 |
| biological process | | | | | |
| (GO:0050789) | | | | | |
| positive regulation | 0.564494 | 0.607273 | 0.613479 | 0.589805 | 0.645453 |
| of cellular process | | | | | |
| (GO:0048522) | | | | | |
| kidney development | 0.613892 | 0.613886 | 0.614729 | 0.555167 | 0.610239 |
| (GO:0001822) | | | | | |
| positive regulation | 0.591363 | 0.597759 | 0.595051 | 0.685149 | 0.616715 |
| of biological | | | | | |
| process | | | | | |
| (GO:0048518) | | | | | |
| multicellular | 0.338896 | 0.612442 | 0.595772 | 0.627888 | 0.600388 |
| organism | | | | | |
| development | | | | | |
| (GO:0007275) | | | | | |
| organic substance | 0.611417 | 0.612524 | 0.613797 | 0.616149 | 0.5468 |
| transport | | | | | |
| (GO:0071702) | | | | | |
| localization | 0.613635 | 0.613634 | 0.614477 | 0.572704 | 0.611717 |
| (GO:0051179) | | | | | |
| establishment of | 0.706892 | 0.734576 | 0.716747 | 0.718748 | 0.737804 |
| localization in cell | | | | | |
| (GO:0051649) | | | | | |
| intracellular signal | 0.496514 | 0.589659 | 0.540428 | 0.698743 | 0.558257 |
| transduction | | | | | |
| (GO:0035556) | | | | | |
| cellular response to | 0.582608 | 0.58788 | 0.584895 | 0.633865 | 0.532492 |
| stimulus | | | | | |
| (GO:0051716) | | | | | |
| nitrogen compound | 0.627021 | 0.704175 | 0.61399 | 0.650931 | 0.599 |
| transport | | | | | |
| (GO:0071705) | | | | | |
| regulation of | 0.544436 | 0.563019 | 0.523398 | 0.550543 | 0.674925 |
| cellular process | | | | | |
| (GO:0050794) | | | | | |
| signaling | 0.612306 | 0.612908 | 0.613159 | 0.608482 | 0.5672 |
| (GO:0023052) | | | | | |
| multicellular | 0.591714 | 0.593125 | 0.583696 | 0.630869 | 0.643058 |
| organism | | | | | |
| development | | | | | |
| (GO:0007275) | | | | | |
| positive regulation | 0.615173 | 0.61516 | 0.615986 | 0.580829 | 0.599971 |
| of cell motility | | | | | |
| (GO:2000147) | | | | | |
| cell communication | 0.68726 | 0.625638 | 0.684902 | 0.708306 | 0.728196 |
| (GO:0007154) | | | | | |
| protein transport | 0.558983 | 0.578577 | 0.59623 | 0.557689 | 0.669758 |
| (GO:0015031) | | | | | |
| organonitrogen | 0.612946 | 0.612933 | 0.61395 | 0.614896 | 0.634462 |
| compound | | | | | |
| metabolic process | | | | | |
| (GO:1901564) | | | | | |
| system | 0.543318 | 0.6116 | 0.558267 | 0.627033 | 0.616556 |
| development | | | | | |
| (GO:0048731) | | | | | |
| regulation of | 0.604843 | 0.604131 | 0.606625 | 0.625237 | 0.648062 |
| localization | | | | | |
| (GO:0032879) | | | | | |
| regulation of | 0.578834 | 0.573951 | 0.57917 | 0.656707 | 0.703893 |
| nitrogen compound | | | | | |
| metabolic process | | | | | |
| (GO:0051171) | | | | | |
| anatomical structure | 0.721733 | 0.62308 | 0.613804 | 0.604421 | 0.671128 |
| morphogenesis | | | | | |
| (GO:0009653) | | | | | |
| regulation of | 0.61366 | 0.613657 | 0.614501 | 0.563446 | 0.60771 |
| primary metabolic | | | | | |
| process | | | | | |
| (GO:0080090) | | | | | |
| macromolecule | 0.59571 | 0.605499 | 0.607064 | 0.578188 | 0.665781 |
| modification | | | | | |
| (GO:0043412) | | | | | |
| multicellular | 0.742741 | 0.718396 | 0.623069 | 0.598685 | 0.664268 |
| organismal process | | | | | |
| (GO:0032501) | | | | | |
| regulation of | 0.613735 | 0.613817 | 0.615652 | 0.677938 | 0.630623 |
| signaling | | | | | |
| (GO:0023051) | | | | | |
| establishment of | 0.603356 | 0.610359 | 0.60513 | 0.361114 | 0.623256 |
| localization | | | | | |
| (GO:0051234) | | | | | |
| response to stimulus | 0.559136 | 0.561078 | 0.580423 | 0.655968 | 0.635996 |
| (GO:0050896) | | | | | |
| regulation of | 0.624402 | 0.61295 | 0.625606 | 0.586602 | 0.588349 |
| signaling | | | | | |
| (GO:0023051) | | | | | |
| regulation of cell | 0.680882 | 0.613582 | 0.715657 | 0.577806 | 0.687156 |
| communication | | | | | |
| (GO:0010646) | | | | | |
| regulation of | 0.624141 | 0.624513 | 0.625012 | 0.618898 | 0.642834 |
| transport | | | | | |
| (GO:0051049) | | | | | |
| nervous system | 0.602255 | 0.602814 | 0.606188 | 0.614868 | 0.62326 |
| development | | | | | |
| (GO:0007399) | | | | | |
| intracellular | 0.650931 | 0.65554 | 0.653105 | 0.660088 | 0.661789 |
| transport | | | | | |
| (GO:0046907) | | | | | |
| organelle | 0.619577 | 0.63981 | 0.624438 | 0.761473 | 0.767724 |
| organization | | | | | |
| (GO:0006996) | | | | | |
| regulation of | 0.613675 | 0.613672 | 0.614518 | 0.594769 | 0.615114 |
| secretion | | | | | |
| (GO:0051046) | | | | | |
| intracellular protein | 0.661664 | 0.667271 | 0.665623 | 0.666034 | 0.699898 |
| transport | | | | | |
| (GO:0006886) | | | | | |
| regulation of signal | 0.565219 | 0.554535 | 0.539489 | 0.615118 | 0.60357 |
| transduction | | | | | |
| (GO:0009966) | | | | | |
| signal transduction | 0.648612 | 0.625339 | 0.641935 | 0.622288 | 0.569731 |
| (GO:0007165) | | | | | |
| protein | 0.512652 | 0.604953 | 0.459299 | 0.642201 | 0.652975 |
| modification | | | | | |
| process | | | | | |
| (GO:0036211) | | | | | |
| secretion | 0.612974 | 0.612974 | 0.613862 | 0.588488 | 0.638776 |
| (GO:0046903) | | | | | |
| regulation of signal | 0.665512 | 0.613112 | 0.624174 | 0.514221 | 0.646315 |
| transduction | | | | | |
| (GO:0009966) | | | | | |
| regulation of | 0.612996 | 0.612996 | 0.61388 | 0.611343 | 0.645657 |
| cellular localization | | | | | |
| (GO:0060341) | | | | | |
| regulation of | 0.613084 | 0.613084 | 0.613969 | 0.603216 | 0.510216 |
| protein localization | | | | | |
| (GO:0032880) | | | | | |
| protein metabolic | 0.613673 | 0.61367 | 0.614515 | 0.619576 | 0.583567 |
| process | | | | | |
| (GO:0019538) | | | | | |
| export from cell | 0.612952 | 0.612952 | 0.613841 | 0.600317 | 0.649564 |
| (GO:0140352) | | | | | |
| vesicle-mediated | 0.583118 | 0.566564 | 0.528554 | 0.671785 | 0.614756 |
| transport | | | | | |
| (GO:0016192) | | | | | |
| establishment of | 0.613938 | 0.619593 | 0.620654 | 0.697303 | 0.705715 |
| protein localization | | | | | |
| (GO:0045184) | | | | | |
| regulation of | 0.613401 | 0.613399 | 0.614245 | 0.534791 | 0.57875 |
| multicellular | | | | | |
| organismal process | | | | | |
| (GO:0051239) | | | | | |
| regulation of | 0.61405 | 0.61405 | 0.614894 | 0.601352 | 0.614713 |
| protein metabolic | | | | | |
| process | | | | | |
| (GO:0051246) | | | | | |
| regulation of | 0.642161 | 0.64214 | 0.641794 | 0.627015 | 0.647096 |
| cellular component | | | | | |
| organization | | | | | |
| (GO:0051128) | | | | | |
| positive regulation | 0.613063 | 0.613063 | 0.613949 | 0.603833 | 0.633164 |
| of protein | | | | | |
| localization | | | | | |
| (GO:1903829) | | | | | |
| cell projection | 0.59064 | 0.582116 | 0.584529 | 0.584255 | 0.633848 |
| organization | | | | | |
| (GO:0030030) | | | | | |
| regulation of | 0.613447 | 0.613445 | 0.614292 | 0.645961 | 0.645277 |
| secretion by cell | | | | | |
| (GO:1903530) | | | | | |
| secretion by cell | 0.612908 | 0.612908 | 0.613796 | 0.59733 | 0.653455 |
| (GO:0032940) | | | | | |
| regulation of | 0.585674 | 0.5772 | 0.584162 | 0.626223 | 0.648826 |
| biological quality | | | | | |
| (GO:0065008) | | | | | |
| positive regulation | 0.612322 | 0.602663 | 0.602815 | 0.515737 | 0.580883 |
| of nitrogen | | | | | |
| compound | | | | | |
| metabolic process | | | | | |
| (GO:0051173) | | | | | |
| cell differentiation | 0.570267 | 0.607764 | 0.61375 | 0.631364 | 0.602266 |
| (GO:0030154) | | | | | |
| negative regulation | 0.518261 | 0.511358 | 0.518887 | 0.619642 | 0.589952 |
| of nitrogen | | | | | |
| compound | | | | | |
| metabolic process | | | | | |
| (GO:0051172) | | | | | |
| positive regulation | 0.618462 | 0.618175 | 0.622157 | 0.664139 | 0.678822 |
| of cellular process | | | | | |
| (GO:0048522) | | | | | |
| plasma membrane | 0.612924 | 0.612138 | 0.573081 | 0.571578 | 0.614428 |
| bounded cell | | | | | |
| projection | | | | | |
| organization | | | | | |
| (GO:0120036) | | | | | |
| phosphorus | 0.592183 | 0.577823 | 0.585623 | 0.518986 | 0.581287 |
| metabolic process | | | | | |
| (GO:0006793) | | | | | |
| anterior/posterior | 0.601465 | 0.53599 | 0.610839 | 0.624222 | 0.679034 |
| pattern specification | | | | | |
| (GO:0009952) | | | | | |
| regulation of | 0.645255 | 0.639603 | 0.660297 | 0.663449 | 0.69143 |
| response to stimulus | | | | | |
| (GO:0048583) | | | | | |
| cellular response to | 0.613663 | 0.61366 | 0.614502 | 0.531293 | 0.639287 |
| stress | | | | | |
| (GO:0033554) | | | | | |
| tissue development | 0.648724 | 0.642779 | 0.643804 | 0.626928 | 0.64538 |
| (GO:0009888) | | | | | |
| transport along | 0.663397 | 0.65694 | 0.641621 | 0.731419 | 0.786074 |
| microtubule | | | | | |
| (GO:0010970) | | | | | |
| positive regulation | 0.650069 | 0.613089 | 0.798306 | 0.654087 | 0.720137 |
| of metabolic | | | | | |
| process | | | | | |
| (GO:0009893) | | | | | |
| phosphate- | 0.592183 | 0.577823 | 0.585623 | 0.52887 | 0.565853 |
| containing | | | | | |
| compound | | | | | |
| metabolic process | | | | | |
| (GO:0006796) | | | | | |
| nucleobase- | 0.594833 | 0.532208 | 0.517181 | 0.463501 | 0.632811 |
| containing | | | | | |
| compound | | | | | |
| biosynthetic process | | | | | |
| (GO:0034654) | | | | | |
| signaling | 0.640422 | 0.636253 | 0.651517 | 0.677574 | 0.666163 |
| (GO:0023052) | | | | | |
| organic cyclic | 0.536863 | 0.453462 | 0.508163 | 0.64789 | 0.705985 |
| compound | | | | | |
| biosynthetic process | | | | | |
| (GO:1901362) | | | | | |
| heterocycle | 0.613018 | 0.613739 | 0.614508 | 0.615895 | 0.57926 |
| biosynthetic process | | | | | |
| (GO:0018130) | | | | | |
| microtubule-based | 0.6849 | 0.68524 | 0.645936 | 0.640275 | 0.673631 |
| transport | | | | | |
| (GO:0099111) | | | | | |
| response to nitrogen | 0.614307 | 0.614314 | 0.615149 | 0.655818 | 0.69006 |
| compound | | | | | |
| (GO:1901698) | | | | | |
| aromatic compound | 0.613018 | 0.663601 | 0.723767 | 0.646719 | 0.560625 |
| biosynthetic process | | | | | |
| (GO:0019438) | | | | | |
| positive regulation | 0.586873 | 0.589588 | 0.590596 | 0.633219 | 0.665985 |
| ofcell | | | | | |
| communication | | | | | |
| (GO:0010647) | | | | | |
| regulation of | 0.595625 | 0.598286 | 0.597977 | 0.660251 | 0.572018 |
| intracellular signal | | | | | |
| transduction | | | | | |
| (GO:1902531) | | | | | |
| positive regulation | 0.612919 | 0.612919 | 0.613804 | 0.626551 | 0.645071 |
| of protein transport | | | | | |
| (GO:0051222) | | | | | |
| regulation of | 0.559651 | 0.604529 | 0.601715 | 0.650744 | 0.673638 |
| establishment of | | | | | |
| protein localization | | | | | |
| (GO:0070201) | | | | | |
| positive regulation | 0.595023 | 0.591425 | 0.594886 | 0.626999 | 0.676436 |
| of establishment of | | | | | |
| protein localization | | | | | |
| (GO:1904951) | | | | | |
| regulation of cell | 0.609884 | 0.611234 | 0.610434 | 0.616109 | 0.679496 |
| differentiation | | | | | |
| (GO:0045595) | | | | | |
| catabolic process | 0.583198 | 0.571406 | 0.581987 | 0.54014 | 0.645373 |
| (GO:0009056) | | | | | |
| positive regulation | 0.604165 | 0.483828 | 0.473577 | 0.492943 | 0.685799 |
| of cellular | | | | | |
| metabolic process | | | | | |
| (GO:0031325) | | | | | |
| tongue development | 0.548345 | 0.554787 | 0.543709 | 0.804353 | 0.753752 |
| (GO:0043586) | | | | | |
| positive regulation | 0.552476 | 0.144881 | 0.555214 | 0.574301 | 0.549922 |
| of transport | | | | | |
| (GO:0051050) | | | | | |
| cytoskeleton- | 0.572552 | 0.577961 | 0.576792 | 0.709366 | 0.678719 |
| dependent | | | | | |
| intracellular | | | | | |
| transport | | | | | |
| (GO:0030705) | | | | | |
| response to | 0.455128 | 0.55712 | 0.429984 | 0.656113 | 0.691653 |
| endogenous | | | | | |
| stimulus | | | | | |
| (GO:0009719) | | | | | |
| regulation of | 0.614946 | 0.626453 | 0.65469 | 0.610022 | 0.644278 |
| protein transport | | | | | |
| (GO:0051223) | | | | | |
| positive regulation | 0.586873 | 0.589588 | 0.590596 | 0.707085 | 0.546791 |
| of signaling | | | | | |
| (GO:0023056) | | | | | |
| regulation of | 0.612445 | 0.575014 | 0.421408 | 0.584497 | 0.667064 |
| nucleobase- | | | | | |
| containing | | | | | |
| compound | | | | | |
| metabolic process | | | | | |
| (GO:0019219) | | | | | |
| regulation of | 0.686488 | 0.713167 | 0.662983 | 0.650773 | 0.644002 |
| cellular component | | | | | |
| size (GO:0032535) | | | | | |
| signal transduction | 0.639515 | 0.635275 | 0.64947 | 0.671847 | 0.664449 |
| (GO:0007165) | | | | | |
| cell development | 0.58516 | 0.581077 | 0.580608 | 0.571609 | 0.613328 |
| (GO:0048468) | | | | | |
| positive regulation | 0.61292 | 0.612573 | 0.603585 | 0.686832 | 0.643275 |
| of macromolecule | | | | | |
| metabolic process | | | | | |
| (GO:0010604) | | | | | |
| positive regulation | 0.644749 | 0.702778 | 0.749409 | 0.52031 | 0.548491 |
| of transcription by | | | | | |
| RNA polymerase II | | | | | |
| (GO:0045944) | | | | | |
| negative regulation | 0.602526 | 0.599931 | 0.602952 | 0.595726 | 0.628028 |
| of biological | | | | | |
| process | | | | | |
| (GO:0048519) | | | | | |
| intracellular | 0.665678 | 0.61295 | 0.692403 | 0.528036 | 0.593885 |
| signaling cassette | | | | | |
| (GO:0141124) | | | | | |
| regulation of | 0.664631 | 0.655046 | 0.645993 | 0.674689 | 0.677044 |
| developmental | | | | | |
| process | | | | | |
| (GO:0050793) | | | | | |
| negative regulation | 0.596735 | 0.592241 | 0.596645 | 0.602515 | 0.628912 |
| of cellular process | | | | | |
| (GO:0048523) | | | | | |
| pattern specification | 0.622976 | 0.648285 | 0.613884 | 0.639617 | 0.656687 |
| process | | | | | |
| (GO:0007389) | | | | | |
| positive regulation | 0.617752 | 0.617358 | 0.618397 | 0.646864 | 0.665031 |
| of signal | | | | | |
| transduction | | | | | |
| (GO:0009967) | | | | | |
| positive regulation | 0.593538 | 0.599382 | 0.597293 | 0.672712 | 0.626691 |
| of intracellular | | | | | |
| signal transduction | | | | | |
| (GO:1902533) | | | | | |
| regulation of | 0.60539 | 0.605548 | 0.603317 | 0.635306 | 0.616181 |
| response to stimulus | | | | | |
| (GO:0048583) | | | | | |
| embryo | 0.645029 | 0.692894 | 0.655162 | 0.626849 | 0.649151 |
| development | | | | | |
| (GO:0009790) | | | | | |
| response to peptide | 0.615335 | 0.615347 | 0.616175 | 0.612997 | 0.648044 |
| (GO:1901652) | | | | | |
| cellular localization | 0.590825 | 0.574351 | 0.577589 | 0.639405 | 0.697315 |
| (GO:0051641) | | | | | |
| biological | 0.585822 | 0.571549 | 0.571447 | 0.649092 | 0.682888 |
| regulation | | | | | |
| (GO:0065007) | | | | | |
| regulation of | 0.583657 | 0.57035 | 0.570204 | 0.643064 | 0.670523 |
| biological process | | | | | |
| (GO:0050789) | | | | | |
| regulation of | 0.586391 | 0.574631 | 0.573917 | 0.656933 | 0.687802 |
| cellular process | | | | | |
| (GO:0050794) | | | | | |
| organelle | 0.612913 | 0.608332 | 0.601107 | 0.633773 | 0.717716 |
| organization | | | | | |
| (GO:0006996) | | | | | |
| positive regulation | 0.577215 | 0.562534 | 0.564118 | 0.599279 | 0.640225 |
| of biological | | | | | |
| process | | | | | |
| (GO:0048518) | | | | | |
| regulation of | 0.597912 | 0.593892 | 0.595418 | 0.67325 | 0.677725 |
| cellular component | | | | | |
| organization | | | | | |
| (GO:0051128) | | | | | |
| localization | 0.599244 | 0.586925 | 0.588773 | 0.580548 | 0.615017 |
| (GO:0051179) | | | | | |
| positive regulation | 0.611255 | 0.611334 | 0.611364 | 0.651143 | 0.6656 |
| of biological | | | | | |
| process | | | | | |
| (GO:0048518) | | | | | |
| regulation of | 0.600791 | 0.599021 | 0.598522 | 0.562957 | 0.608829 |
| nitrogen compound | | | | | |
| metabolic process | | | | | |
| (GO:0051171) | | | | | |
| cell cycle | 0.601696 | 0.598667 | 0.598799 | 0.632142 | 0.656905 |
| (GO:0007049) | | | | | |
| positive regulation | 0.586606 | 0.57256 | 0.574835 | 0.593146 | 0.622558 |
| of cellular process | | | | | |
| (GO:0048522) | | | | | |
| protein | 0.612908 | 0.612769 | 0.571977 | 0.675912 | 0.716803 |
| modification | | | | | |
| process | | | | | |
| (GO:0036211) | | | | | |
| mitotic cell cycle | 0.576597 | 0.561194 | 0.563831 | 0.559412 | 0.593341 |
| process | | | | | |
| (GO:1903047) | | | | | |
| regulation of | 0.604032 | 0.602453 | 0.602431 | 0.617344 | 0.450187 |
| primary metabolic | | | | | |
| process | | | | | |
| (GO:0080090) | | | | | |
| macromolecule | 0.587777 | 0.578101 | 0.579728 | 0.659107 | 0.725151 |
| modification | | | | | |
| (GO:0043412) | | | | | |
| cell cycle process | 0.581661 | 0.566316 | 0.568167 | 0.653408 | 0.65458 |
| (GO:0022402) | | | | | |
| protein metabolic | 0.612739 | 0.441807 | 0.481673 | 0.667286 | 0.699451 |
| process | | | | | |
| (GO:0019538) | | | | | |
| mitotic cell cycle | 0.581504 | 0.562212 | 0.565234 | 0.546544 | 0.589883 |
| (GO:0000278) | | | | | |
| anatomical structure | 0.613937 | 0.613929 | 0.61476 | 0.572022 | 0.613393 |
| morphogenesis | | | | | |
| (GO:0009653) | | | | | |
| cell population | 0.552586 | 0.535445 | 0.524946 | 0.577443 | 0.614618 |
| proliferation | | | | | |
| (GO:0008283) | | | | | |
| cellular | 0.612988 | 0.61303 | 0.613861 | 0.625006 | 0.684754 |
| macromolecule | | | | | |
| localization | | | | | |
| (GO:0070727) | | | | | |
| protein localization | 0.61041 | 0.607786 | 0.609133 | 0.641293 | 0.701659 |
| (GO:0008104) | | | | | |
| organonitrogen | 0.613362 | 0.628335 | 0.629165 | 0.647783 | 0.712701 |
| compound | | | | | |
| metabolic process | | | | | |
| (GO:1901564) | | | | | |
| catabolic process | 0.572726 | 0.555443 | 0.562401 | 0.621062 | 0.639749 |
| (GO:0009056) | | | | | |
| phosphorylation | 0.593588 | 0.581115 | 0.582567 | 0.575159 | 0.646887 |
| (GO:0016310) | | | | | |
| establishment of | 0.593635 | 0.579061 | 0.578563 | 0.624202 | 0.695788 |
| localization | | | | | |
| (GO:0051234) | | | | | |
| macromolecule | 0.615304 | 0.617752 | 0.618366 | 0.494273 | 0.604649 |
| localization | | | | | |
| (GO:0033036) | | | | | |
| regulation of | 0.593996 | 0.590102 | 0.590545 | 0.648267 | 0.647398 |
| metabolic process | | | | | |
| (GO:0019222) | | | | | |
| cellular response to | 0.612947 | 0.605568 | 0.605323 | 0.581713 | 0.606343 |
| stimulus | | | | | |
| (GO:0051716) | | | | | |
| establishment of | 0.606164 | 0.601795 | 0.603117 | 0.717541 | 0.733755 |
| localization in cell | | | | | |
| (GO:0051649) | | | | | |
| connective tissue | 0.588533 | 0.58357 | 0.586609 | 0.651732 | 0.628374 |
| development | | | | | |
| (GO:0061448) | | | | | |
| transport | 0.60599 | 0.601029 | 0.601185 | 0.577571 | 0.62198 |
| (GO:0006810) | | | | | |
| cellular response to | 0.63909 | 0.6464 | 0.645055 | 0.686189 | 0.6858 |
| stress | | | | | |
| (GO:0033554) | | | | | |
| positive regulation | 0.595768 | 0.590792 | 0.591901 | 0.586894 | 0.616272 |
| of metabolic | | | | | |
| process | | | | | |
| (GO:0009893) | | | | | |
| phosphate- | 0.604734 | 0.611304 | 0.57727 | 0.562182 | 0.440531 |
| containing | | | | | |
| compound | | | | | |
| metabolic process | | | | | |
| (GO:0006796) | | | | | |
| regulation of | 0.610568 | 0.609404 | 0.610241 | 0.663069 | 0.669836 |
| organelle | | | | | |
| organization | | | | | |
| (GO:0033043) | | | | | |
| regulation of | 0.608692 | 0.567182 | 0.496004 | 0.673796 | 0.701785 |
| cellular component | | | | | |
| biogenesis | | | | | |
| (GO:0044087) | | | | | |
| regulation of | 0.58169 | 0.57318 | 0.574106 | 0.638852 | 0.663339 |
| cellular metabolic | | | | | |
| process | | | | | |
| (GO:0031323) | | | | | |
| phosphorus | 0.612698 | 0.612897 | 0.61329 | 0.563662 | 0.681243 |
| metabolic process | | | | | |
| (GO:0006793) | | | | | |
| cell division | 0.613405 | 0.613399 | 0.614241 | 0.621353 | 0.618896 |
| (GO:0051301) | | | | | |
| regulation of | 0.582082 | 0.577845 | 0.57704 | 0.626111 | 0.640128 |
| nucleobase- | | | | | |
| containing | | | | | |
| compound | | | | | |
| metabolic process | | | | | |
| (GO:0019219) | | | | | |
| epithelial cell | 0.612804 | 0.612804 | 0.613694 | 0.700178 | 0.664643 |
| proliferation | | | | | |
| (GO:0050673) | | | | | |
| autophagy | 0.613029 | 0.681464 | 0.68648 | 0.598636 | 0.611588 |
| (GO:0006914) | | | | | |
| mitotic nuclear | 0.613447 | 0.613438 | 0.614285 | 0.591874 | 0.662407 |
| division | | | | | |
| (GO:0140014) | | | | | |
| regulation of | 0.532556 | 0.536421 | 0.499711 | 0.688122 | 0.653685 |
| macromolecule | | | | | |
| metabolic process | | | | | |
| (GO:0060255) | | | | | |
| regulation of cell | 0.613651 | 0.613644 | 0.614486 | 0.65198 | 0.674557 |
| cycle | | | | | |
| (GO:0051726) | | | | | |
| nuclear division | 0.612854 | 0.599051 | 0.600592 | 0.639879 | 0.638664 |
| (GO:0000280) | | | | | |
| vesicle-mediated | 0.615025 | 0.621195 | 0.629566 | 0.706116 | 0.665624 |
| transport | | | | | |
| (GO:0016192) | | | | | |
| regulation of | 0.59509 | 0.580096 | 0.580476 | 0.723519 | 0.645119 |
| response to stimulus | | | | | |
| (GO:0048583) | | | | | |
| positive regulation | 0.595187 | 0.59079 | 0.591574 | 0.658538 | 0.680933 |
| of macromolecule | | | | | |
| metabolic process | | | | | |
| (GO:0010604) | | | | | |
| nucleobase- | 0.582157 | 0.576459 | 0.57654 | 0.575114 | 0.603834 |
| containing | | | | | |
| compound | | | | | |
| biosynthetic process | | | | | |
| (GO:0034654) | | | | | |
| protein | 0.622018 | 0.629488 | 0.629813 | 0.696665 | 0.648154 |
| phosphorylation | | | | | |
| (GO:0006468) | | | | | |
| positive regulation | 0.564887 | 0.572068 | 0.550068 | 0.618465 | 0.650263 |
| of developmental | | | | | |
| process | | | | | |
| (GO:0051094) | | | | | |
| organelle | 0.640212 | 0.634976 | 0.661968 | 0.591091 | 0.673479 |
| localization | | | | | |
| (GO:0051640) | | | | | |
| positive regulation | 0.595202 | 0.595729 | 0.581183 | 0.612625 | 0.663367 |
| of nitrogen | | | | | |
| compound | | | | | |
| metabolic process | | | | | |
| (GO:0051173) | | | | | |
| positive regulation | 0.583805 | 0.579196 | 0.582439 | 0.601629 | 0.633239 |
| of cellular | | | | | |
| component | | | | | |
| organization | | | | | |
| (GO:0051130) | | | | | |
| aromatic compound | 0.587081 | 0.582375 | 0.582644 | 0.57452 | 0.605667 |
| biosynthetic process | | | | | |
| (GO:0019438) | | | | | |
| heterocycle | 0.585593 | 0.580719 | 0.580878 | 0.574922 | 0.606876 |
| biosynthetic process | | | | | |
| (GO:0018130) | | | | | |
| cell cycle phase | 0.610133 | 0.609154 | 0.610196 | 0.575108 | 0.599878 |
| transition | | | | | |
| (GO:0044770) | | | | | |
| regulation of signal | 0.616324 | 0.618899 | 0.619764 | 0.663763 | 0.687448 |
| transduction | | | | | |
| (GO:0009966) | | | | | |
| RNA biosynthetic | 0.568617 | 0.559086 | 0.558657 | 0.553845 | 0.586039 |
| process | | | | | |
| (GO:0032774) | | | | | |
| protein transport | 0.63309 | 0.649713 | 0.651872 | 0.789967 | 0.628707 |
| (GO:0015031) | | | | | |
| DNA-templated | 0.568947 | 0.560455 | 0.559609 | 0.557931 | 0.625142 |
| transcription | | | | | |
| (GO:0006351) | | | | | |
| regulation of | 0.687007 | 0.682564 | 0.728467 | 0.708417 | 0.724057 |
| cellular process | | | | | |
| (GO:0050794) | | | | | |
| organic cyclic | 0.591523 | 0.587344 | 0.587472 | 0.579363 | 0.607821 |
| compound | | | | | |
| biosynthetic process | | | | | |
| (GO:1901362) | | | | | |
| chromosome | 0.646899 | 0.666679 | 0.66391 | 0.699995 | 0.657595 |
| segregation | | | | | |
| (GO:0007059) | | | | | |
| regulation of | 0.602381 | 0.600138 | 0.601899 | 0.534691 | 0.603344 |
| catabolic process | | | | | |
| (GO:0009894) | | | | | |
| organelle fission | 0.579118 | 0.562741 | 0.567616 | 0.756579 | 0.540637 |
| (GO:0048285) | | | | | |
| positive regulation | 0.587315 | 0.587026 | 0.58783 | 0.581713 | 0.59352 |
| of cellular | | | | | |
| component | | | | | |
| biogenesis | | | | | |
| (GO:0044089) | | | | | |
| regulation of RNA | 0.572746 | 0.566335 | 0.563741 | 0.558401 | 0.765913 |
| biosynthetic process | | | | | |
| (GO:2001141) | | | | | |
| positive regulation | 0.599635 | 0.595997 | 0.597337 | 0.601113 | 0.639413 |
| of cellular | | | | | |
| metabolic process | | | | | |
| (GO:0031325) | | | | | |
| nitrogen compound | 0.630519 | 0.639374 | 0.641582 | 0.600379 | 0.618307 |
| transport | | | | | |
| (GO:0071705) | | | | | |
| mitotic cell cycle | 0.600046 | 0.59566 | 0.597697 | 0.618226 | 0.634048 |
| phase transition | | | | | |
| (GO:0044772) | | | | | |
| regulation of DNA- | 0.575639 | 0.570121 | 0.56746 | 0.559237 | 0.597019 |
| templated | | | | | |
| transcription | | | | | |
| (GO:0006355) | | | | | |
| cellular response to | 0.655469 | 0.642276 | 0.670908 | 0.687237 | 0.682927 |
| stimulus | | | | | |
| (GO:0051716) | | | | | |
| regulation of RNA | 0.580966 | 0.574943 | 0.574042 | 0.562392 | 0.603763 |
| metabolic process | | | | | |
| (GO:0051252) | | | | | |
| positive regulation | 0.597418 | 0.59252 | 0.595427 | 0.591088 | 0.639142 |
| of organelle | | | | | |
| organization | | | | | |
| (GO:0010638) | | | | | |
| response to stimulus | 0.5895 | 0.571611 | 0.571812 | 0.568898 | 0.609472 |
| (GO:0050896) | | | | | |
| cytoskeleton | 0.649261 | 0.660427 | 0.661242 | 0.693079 | 0.664405 |
| organization | | | | | |
| (GO:0007010) | | | | | |
| regulation of | 0.61294 | 0.612232 | 0.586704 | 0.584041 | 0.621816 |
| signaling | | | | | |
| (GO:0023051) | | | | | |
| mitotic sister | 0.613003 | 0.613002 | 0.613845 | 0.648311 | 0.642504 |
| chromatid | | | | | |
| segregation | | | | | |
| (GO:0000070) | | | | | |
| regulation of cell | 0.607325 | 0.603518 | 0.60355 | 0.583967 | 0.622035 |
| communication | | | | | |
| (GO:0010646) | | | | | |
| macroautophagy | 0.586038 | 0.563848 | 0.518837 | 0.602092 | 0.591499 |
| (GO:0016236) | | | | | |
| nuclear | 0.613268 | 0.613263 | 0.61411 | 0.600542 | 0.598445 |
| chromosome | | | | | |
| segregation | | | | | |
| (GO:0098813) | | | | | |
| establishment of | 0.613861 | 0.613852 | 0.614687 | 0.521857 | 0.516265 |
| organelle | | | | | |
| localization | | | | | |
| (GO:0051656) | | | | | |
| regulation of | 0.612713 | 0.612713 | 0.613601 | 0.598019 | 0.658144 |
| osteoblast | | | | | |
| differentiation | | | | | |
| (GO:0045667) | | | | | |
| cellular catabolic | 0.540785 | 0.540793 | 0.541662 | 0.56304 | 0.627061 |
| process | | | | | |
| (GO:0044248) | | | | | |
| organic substance | 0.626734 | 0.639203 | 0.640466 | 0.590384 | 0.615645 |
| transport | | | | | |
| (GO:0071702) | | | | | |
| transcription by | 0.562815 | 0.555637 | 0.55357 | 0.641674 | 0.642794 |
| RNA polymerase II | | | | | |
| (GO:0006366) | | | | | |
| intracellular signal | 0.639635 | 0.650276 | 0.652714 | 0.635872 | 0.66172 |
| transduction | | | | | |
| (GO:0035556) | | | | | |
| establishment of | 0.632779 | 0.64461 | 0.645545 | 0.602621 | 0.619253 |
| protein localization | | | | | |
| (GO:0045184) | | | | | |
| regulation of | 0.58988 | 0.572197 | 0.575525 | 0.568207 | 0.598161 |
| biological quality | | | | | |
| (GO:0065008) | | | | | |
| regulation of | 0.612917 | 0.612789 | 0.61153 | 0.626559 | 0.642441 |
| transcription by | | | | | |
| RNA polymerase II | | | | | |
| (GO:0006357) | | | | | |
| response to stress | 0.592561 | 0.58597 | 0.585778 | 0.558079 | 0.617164 |
| (GO:0006950) | | | | | |
| chromosome | 0.61351 | 0.613502 | 0.614347 | 0.599673 | 0.647276 |
| organization | | | | | |
| (GO:0051276) | | | | | |
| regulation of | 0.556114 | 0.53195 | 0.531737 | 0.709706 | 0.750943 |
| localization | | | | | |
| (GO:0032879) | | | | | |
| regulation of | 0.613234 | 0.613231 | 0.614068 | 0.613522 | 0.62698 |
| anatomical structure | | | | | |
| morphogenesis | | | | | |
| (GO:0022603) | | | | | |
| regulation of | 0.655989 | 0.67206 | 0.672176 | 0.669177 | 0.70463 |
| protem | | | | | |
| modification | | | | | |
| process | | | | | |
| (GO:0031399) | | | | | |
| regulation of | 0.638678 | 0.644502 | 0.651933 | 0.635894 | 0.66174 |
| protein metabolic | | | | | |
| process | | | | | |
| (GO:0051246) | | | | | |
| multicellular | 0.579205 | 0.570599 | 0.569629 | 0.609528 | 0.639192 |
| organism | | | | | |
| development | | | | | |
| (GO:0007275) | | | | | |
| intracellular | 0.628326 | 0.638342 | 0.637617 | 0.624735 | 0.639696 |
| transport | | | | | |
| (GO:0046907) | | | | | |
| regulation of cell | 0.576123 | 0.555658 | 0.555276 | 0.584825 | 0.617263 |
| cycle process | | | | | |
| (GO:0010564) | | | | | |
| DNA metabolic | 0.58411 | 0.582422 | 0.58404 | 0.71793 | 0.708132 |
| process | | | | | |
| (GO:0006259) | | | | | |
| cellular nitrogen | 0.590983 | 0.58199 | 0.583159 | 0.794151 | 0.620212 |
| compound | | | | | |
| biosynthetic process | | | | | |
| (GO:0044271) | | | | | |
| membrane | 0.707999 | 0.747054 | 0.747393 | 0.649362 | 0.699237 |
| organization | | | | | |
| (GO:0061024) | | | | | |
| DNA damage | 0.571077 | 0.567704 | 0.568953 | 0.555564 | 0.657828 |
| response | | | | | |
| (GO:0006974) | | | | | |
| sister chromatid | 0.61309 | 0.613088 | 0.613931 | 0.612266 | 0.620768 |
| segregation | | | | | |
| (GO:0000819) | | | | | |
| regulation of | 0.613672 | 0.613667 | 0.614511 | 0.553646 | 0.593151 |
| multicellular | | | | | |
| organismal | | | | | |
| development | | | | | |
| (GO:2000026) | | | | | |
| regulation of | 0.57305 | 0.573132 | 0.569068 | 0.575978 | 0.651172 |
| molecular function | | | | | |
| (GO:0065009) | | | | | |
| positive regulation | 0.632218 | 0.632306 | 0.652314 | 0.662681 | 0.709037 |
| of nucleobase- | | | | | |
| containing | | | | | |
| compound | | | | | |
| metabolic process | | | | | |
| (GO:0045935) | | | | | |
| macromolecule | 0.566968 | 0.554091 | 0.560539 | 0.620685 | 0.562037 |
| catabolic process | | | | | |
| (GO:0009057) | | | | | |
| nervous system | 0.592456 | 0.591685 | 0.593626 | 0.630014 | 0.693728 |
| development | | | | | |
| (GO:0007399) | | | | | |
| regulation of | 0.626043 | 0.628449 | 0.627674 | 0.560317 | 0.644398 |
| autophagy | | | | | |
| (GO:0010506) | | | | | |
| anatomical structure | 0.596804 | 0.590019 | 0.589046 | 0.624943 | 0.648388 |
| development | | | | | |
| (GO:0048856) | | | | | |
| protein catabolic | 0.579042 | 0.57185 | 0.575481 | 0.620626 | 0.63932 |
| process | | | | | |
| (GO:0030163) | | | | | |
| organic substance | 0.571509 | 0.547112 | 0.556574 | 0.55483 | 0.638882 |
| catabolic process | | | | | |
| (GO:1901575) | | | | | |
| regulation of | 0.579409 | 0.563381 | 0.564631 | 0.547915 | 0.621183 |
| mitotic cell cycle | | | | | |
| (GO:0007346) | | | | | |
| system | 0.583324 | 0.578887 | 0.579696 | 0.701936 | 0.664105 |
| development | | | | | |
| (GO:0048731) | | | | | |
| supramolecular | 0.595533 | 0.591059 | 0.588445 | 0.626411 | 0.601339 |
| fiber organization | | | | | |
| (GO:0097435) | | | | | |
| regulation of | 0.619555 | 0.621061 | 0.621812 | 0.581964 | 0.646649 |
| cellular catabolic | | | | | |
| process | | | | | |
| (GO:0031329) | | | | | |
| developmental | 0.596816 | 0.589867 | 0.589404 | 0.611555 | 0.635525 |
| process | | | | | |
| (GO:0032502) | | | | | |
| vacuole | 0.642051 | 0.649116 | 0.644788 | 0.639981 | 0.659992 |
| organization | | | | | |
| (GO:0007033) | | | | | |
| regulation of | 0.55359 | 0.56463 | 0.555845 | 0.581432 | 0.649903 |
| catalytic activity | | | | | |
| (GO:0050790) | | | | | |
| proteolysis involved | 0.599678 | 0.596851 | 0.599437 | 0.586618 | 0.722927 |
| in protein catabolic | | | | | |
| process | | | | | |
| (GO:0051603) | | | | | |
| regulation of cell | 0.658618 | 0.67097 | 0.673075 | 0.628716 | 0.65446 |
| cycle phase | | | | | |
| transition | | | | | |
| (GO:1901987) | | | | | |
| positive regulation | 0.611517 | 0.611551 | 0.612384 | 0.613162 | 0.650874 |
| of chromosome | | | | | |
| separation | | | | | |
| (GO:1905820) | | | | | |
| regulation of | 0.60293 | 0.601784 | 0.602549 | 0.583523 | 0.620764 |
| cellular component | | | | | |
| size (GO:0032535) | | | | | |
| negative regulation | 0.598617 | 0.591682 | 0.590827 | 0.761121 | 0.638701 |
| of biological | | | | | |
| process | | | | | |
| (GO:0048519) | | | | | |
| regulation of | 0.613467 | 0.613458 | 0.614308 | 0.627821 | 0.721446 |
| mitotic nuclear | | | | | |
| division | | | | | |
| (GO:0007088) | | | | | |
| circulatory system | 0.614201 | 0.614193 | 0.615031 | 0.584395 | 0.599089 |
| development | | | | | |
| (GO:0072359) | | | | | |
| muscle structure | 0.498762 | 0.526104 | 0.498035 | 0.617308 | 0.652938 |
| development | | | | | |
| (GO:0061061) | | | | | |
| organonitrogen | 0.56541 | 0.544426 | 0.552529 | 0.650673 | 0.68793 |
| compound catabolic | | | | | |
| process | | | | | |
| (GO:1901565) | | | | | |
| regulation of | 0.636989 | 0.650628 | 0.653588 | 0.550876 | 0.623525 |
| response to stress | | | | | |
| (GO:0080134) | | | | | |
| regulation of | 0.599626 | 0.58477 | 0.573766 | 0.69686 | 0.650031 |
| mitotic cell cycle | | | | | |
| phase transition | | | | | |
| (GO:1901990) | | | | | |
| regulation of | 0.642914 | 0.668086 | 0.668168 | 0.656297 | 0.68263 |
| intracellular signal | | | | | |
| transduction | | | | | |
| (GO:1902531) | | | | | |
| negative regulation | 0.598994 | 0.59624 | 0.596567 | 0.620911 | 0.639778 |
| of nitrogen | | | | | |
| compound | | | | | |
| metabolic process | | | | | |
| (GO:0051172) | | | | | |
| positive regulation | 0.614486 | 0.615685 | 0.616533 | 0.607527 | 0.640847 |
| of response to | | | | | |
| stimulus | | | | | |
| (GO:0048584) | | | | | |
| regulation of | 0.613202 | 0.613197 | 0.614047 | 0.627819 | 0.654986 |
| nuclear division | | | | | |
| (GO:0051783) | | | | | |
| synaptic vesicle | 0.547551 | 0.612947 | 0.548145 | 0.620441 | 0.639072 |
| cycle | | | | | |
| (GO:0099504) | | | | | |
| negative regulation | 0.611104 | 0.60982 | 0.610332 | 0.623575 | 0.685182 |
| of cellular | | | | | |
| component | | | | | |
| organization | | | | | |
| (GO:0051129) | | | | | |
| positive regulation | 0.610856 | 0.610907 | 0.611729 | 0.615358 | 0.624149 |
| of mitotic sister | | | | | |
| chromatid | | | | | |
| separation | | | | | |
| (GO:1901970) | | | | | |
| regulation of cell | 0.614107 | 0.614095 | 0.614947 | 0.558003 | 0.605405 |
| population | | | | | |
| proliferation | | | | | |
| (GO:0042127) | | | | | |
| regulation of | 0.614528 | 0.613021 | 0.633812 | 0.699186 | 0.681355 |
| protein localization | | | | | |
| (GO:0032880) | | | | | |
| positive regulation | 0.587107 | 0.574242 | 0.580195 | 0.528861 | 0.591783 |
| of catabolic process | | | | | |
| (GO:0009896) | | | | | |
| vesicle-mediated | 0.597944 | 0.612901 | 0.537511 | 0.677146 | 0.602498 |
| transport in synapse | | | | | |
| (GO:0099003) | | | | | |
| proteolysis | 0.61645 | 0.6349 | 0.636022 | 0.710671 | 0.7056 |
| (GO:0006508) | | | | | |
| regulation of | 0.619756 | 0.613169 | 0.618549 | 0.63115 | 0.730242 |
| protein | | | | | |
| phosphorylation | | | | | |
| (GO:0001932) | | | | | |
| regulation of | 0.613329 | 0.613046 | 0.629935 | 0.674176 | 0.700751 |
| cellular localization | | | | | |
| (GO:0060341) | | | | | |
| import into cell | 0.673239 | 0.686677 | 0.710783 | 0.691105 | 0.709577 |
| (GO:0098657) | | | | | |
| cell communication | 0.61957 | 0.69936 | 0.745302 | 0.576365 | 0.608208 |
| (GO:0007154) | | | | | |
| chromosome | 0.612664 | 0.61267 | 0.613508 | 0.599309 | 0.62761 |
| separation | | | | | |
| (GO:0051304) | | | | | |
| regulation of | 0.596923 | 0.592441 | 0.597655 | 0.5962 | 0.615108 |
| chromosome | | | | | |
| separation | | | | | |
| (GO:1905818) | | | | | |
| response to stimulus | 0.662631 | 0.654529 | 0.687282 | 0.684886 | 0.688103 |
| (GO:0050896) | | | | | |
| modification- | 0.589734 | 0.586907 | 0.589487 | 0.589432 | 0.626716 |
| dependent | | | | | |
| imacromolecule | | | | | |
| catabolic process | | | | | |
| (GO:0043632) | | | | | |
| modification- | 0.590521 | 0.587701 | 0.590389 | 0.584701 | 0.53428 |
| dependent protein | | | | | |
| catabolic process | | | | | |
| (GO:0019941) | | | | | |
| regulation of | 0.642991 | 0.646824 | 0.653036 | 0.637538 | 0.671615 |
| anatomical structure | | | | | |
| size (GO:0090066) | | | | | |
| regulation of | 0.61246 | 0.61247 | 0.61331 | 0.618309 | 0.658864 |
| chromosome | | | | | |
| segregation | | | | | |
| (GO:0051983) | | | | | |
| cellular response to | 0.609878 | 0.608886 | 0.609308 | 0.628594 | 0.646995 |
| chemical stimulus | | | | | |
| (GO:0070887) | | | | | |
| actin cytoskeleton | 0.581765 | 0.581396 | 0.581096 | 0.613298 | 0.685703 |
| organization | | | | | |
| (GO:0030036) | | | | | |
| signal transduction | 0.583366 | 0.559524 | 0.55986 | 0.579529 | 0.609929 |
| (GO:0007165) | | | | | |
| signaling | 0.581652 | 0.559306 | 0.558448 | 0.573638 | 0.606519 |
| (GO:0023052) | | | | | |
| TOR signaling | 0.612003 | 0.612112 | 0.611997 | 0.65286 | 0.640542 |
| (GO:0031929) | | | | | |
| peptidyl-amino acid | 0.619342 | 0.619095 | 0.620301 | 0.57539 | 0.625658 |
| modification | | | | | |
| (GO:0018193) | | | | | |
| negative regulation | 0.567746 | 0.562785 | 0.564139 | 0.528701 | 0.571728 |
| of developmental | | | | | |
| process | | | | | |
| (GO:0051093) | | | | | |
| regulation of | 0.600599 | 0.599304 | 0.597496 | 0.599223 | 0.689728 |
| phosphorylation | | | | | |
| (GO:0042325) | | | | | |
| negative regulation | 0.600784 | 0.593036 | 0.592868 | 0.601696 | 0.628337 |
| of cellular process | | | | | |
| (GO:0048523) | | | | | |
| ubiquitin-dependent | 0.585927 | 0.582494 | 0.585212 | 0.582746 | 0.622529 |
| protein catabolic | | | | | |
| process | | | | | |
| (GO:0006511) | | | | | |
| positive regulation | 0.6131 | 0.613099 | 0.613935 | 0.599691 | 0.638915 |
| of autophagy | | | | | |
| (GO:0010508) | | | | | |
| regulation of | 0.636182 | 0.648251 | 0.654018 | 0.61872 | 0.55274 |
| transport | | | | | |
| (GO:0051049) | | | | | |
| positive regulation | 0.640025 | 0.650022 | 0.650197 | 0.696527 | 0.757202 |
| of DNA-templated | | | | | |
| transcription | | | | | |
| (GO:0045893) | | | | | |
| actin filament-based | 0.588732 | 0.588338 | 0.588079 | 0.617369 | 0.613509 |
| process | | | | | |
| (GO:0030029) | | | | | |
| regulation of | 0.643887 | 0.67928 | 0.675891 | 0.601667 | 0.579428 |
| protein-containing | | | | | |
| complex assembly | | | | | |
| (GO:0043254) | | | | | |
| regulation of | 0.664295 | 0.863612 | 0.801906 | 0.611871 | 0.614203 |
| mitotic sister | | | | | |
| chromatid | | | | | |
| separation | | | | | |
| (GO:0010965) | | | | | |
| positive regulation | 0.628622 | 0.638496 | 0.637209 | 0.641855 | 0.697693 |
| of macromolecule | | | | | |
| biosynthetic process | | | | | |
| (GO:0010557) | | | | | |
| collagen fibril | 0.612908 | 0.612908 | 0.613797 | 0.596381 | 0.605899 |
| organization | | | | | |
| (GO:0030199) | | | | | |
| endocytosis | 0.6193 | 0.622023 | 0.624204 | 0.614478 | 0.637525 |
| (GO:0006897) | | | | | |
| protein localization | 0.612947 | 0.519952 | 0.514915 | 0.611905 | 0.700837 |
| to plasma | | | | | |
| membrane | | | | | |
| (GO:0072659) | | | | | |
| positive regulation | 0.630779 | 0.64083 | 0.639523 | 0.644436 | 0.689802 |
| of biosynthetic | | | | | |
| process | | | | | |
| (GO:0009891) | | | | | |
| regulation of cell | 0.604013 | 0.603063 | 0.604225 | 0.618337 | 0.647542 |
| size (GO:0008361) | | | | | |
| positive regulation | 0.627954 | 0.624089 | 0.635743 | 0.685231 | 0.68897 |
| of RNA metabolic | | | | | |
| process | | | | | |
| (GO:0051254) | | | | | |
| TORC1 signaling | 0.615225 | 0.614343 | 0.620883 | 0.556178 | 0.645658 |
| (GO:0038202) | | | | | |
| positive regulation | 0.657491 | 0.657223 | 0.672813 | 0.669964 | 0.659936 |
| of protein metabolic | | | | | |
| process | | | | | |
| (GO:0051247) | | | | | |
| positive regulation | 0.635416 | 0.647904 | 0.646017 | 0.649469 | 0.69835 |
| of cellular | | | | | |
| biosynthetic process | | | | | |
| (GO:0031328) | | | | | |
| microtubule | 0.612962 | 0.612962 | 0.613803 | 0.649643 | 0.646681 |
| cytoskeleton | | | | | |
| organization | | | | | |
| involved in mitosis | | | | | |
| (GO:1902850) | | | | | |
| synaptic vesicle | 0.619908 | 0.617843 | 0.619241 | 0.598652 | 0.613012 |
| recycling | | | | | |
| (GO:0036465) | | | | | |
| cell surface receptor | 0.613128 | 0.613127 | 0.613967 | 0.621572 | 0.623295 |
| signaling pathway | | | | | |
| (GO:0007166) | | | | | |
| chromosome | 0.61307 | 0.613068 | 0.613908 | 0.620088 | 0.649382 |
| localization | | | | | |
| (GO:0050000) | | | | | |
| establishment of | 0.612987 | 0.612986 | 0.613827 | 0.616778 | 0.728059 |
| chromosome | | | | | |
| localization | | | | | |
| (GO:0051303) | | | | | |
| cellular component | 0.584017 | 0.578157 | 0.580075 | 0.668621 | 0.704629 |
| organization | | | | | |
| (GO:0016043) | | | | | |
| intracellular | 0.613059 | 0.613089 | 0.614118 | 0.605971 | 0.650741 |
| signaling cassette | | | | | |
| (GO:0141124) | | | | | |
| protein | 0.594906 | 0.59356 | 0.595728 | 0.656424 | 0.671575 |
| modification by | | | | | |
| small protein | | | | | |
| conjugation | | | | | |
| (GO:0032446) | | | | | |
| regulation of | 0.622016 | 0.612947 | 0.614277 | 0.689162 | 0.7362 |
| TORC1 signaling | | | | | |
| (GO:1903432) | | | | | |
| post-translational | 0.61692 | 0.615766 | 0.696108 | 0.741786 | 0.682683 |
| protein | | | | | |
| modification | | | | | |
| (GO:0043687) | | | | | |
| positive regulation | 0.565343 | 0.546909 | 0.540445 | 0.580005 | 0.587126 |
| of molecular | | | | | |
| function | | | | | |
| (GO:0044093) | | | | | |
| regulation of | 0.594972 | 0.588349 | 0.589677 | 0.634148 | 0.647091 |
| macromolecule | | | | | |
| biosynthetic process | | | | | |
| (GO:0010556) | | | | | |
| proteasomal protein | 0.622053 | 0.627744 | 0.627055 | 0.626817 | 0.614939 |
| catabolic process | | | | | |
| (GO:0010498) | | | | | |
| angiogenesis | 0.528717 | 0.502839 | 0.500489 | 0.598188 | 0.636611 |
| (GO:0001525) | | | | | |
| regulation of | 0.603908 | 0.603786 | 0.605062 | 0.58975 | 0.561773 |
| synapse structure or | | | | | |
| activity | | | | | |
| (GO:0050803) | | | | | |
| negative regulation | 0.621694 | 0.617307 | 0.651042 | 0.625271 | 0.71701 |
| of TOR signaling | | | | | |
| (GO:0032007) | | | | | |
| negative regulation | 0.770075 | 0.827359 | 0.80521 | 0.808769 | 0.736326 |
| of cell cycle phase | | | | | |
| transition | | | | | |
| (GO:1901988) | | | | | |
| positive regulation | 0.664178 | 0.70094 | 0.702317 | 0.723807 | 0.679347 |
| of signaling | | | | | |
| (GO:0023056) | | | | | |
| metaphase | 0.612651 | 0.612656 | 0.613497 | 0.626017 | 0.643559 |
| chromosome | | | | | |
| alignment | | | | | |
| (GO:0051310) | | | | | |
| negative regulation | 0.627277 | 0.635234 | 0.634373 | 0.759636 | 0.692424 |
| of cell cycle process | | | | | |
| (GO:0010948) | | | | | |
| regulation of | 0.54006 | 0.538375 | 0.543208 | 0.684606 | 0.716505 |
| plasma membrane | | | | | |
| bounded cell | | | | | |
| projection assembly | | | | | |
| (GO:0120032) | | | | | |
| cell junction | 0.472203 | 0.539901 | 0.452402 | 0.703183 | 0.730645 |
| organization | | | | | |
| (GO:0034330) | | | | | |
| regulation of | 0.576879 | 0.577694 | 0.579038 | 0.563385 | 0.569289 |
| cellular response to | | | | | |
| stress | | | | | |
| (GO:0080135) | | | | | |
| positive regulation | 0.585247 | 0.586134 | 0.587126 | 0.642115 | 0.628503 |
| of mitotic | | | | | |
| cytokinesis | | | | | |
| (GO:1903490) | | | | | |
| transmembrane | 0.582088 | 0.58061 | 0.580689 | 0.601048 | 0.607419 |
| receptor protein | | | | | |
| tyrosine kinase | | | | | |
| signaling pathway | | | | | |
| (GO:0007169) | | | | | |
| regulation of | 0.5836 | 0.574476 | 0.57427 | 0.563984 | 0.614615 |
| phosphate | | | | | |
| metabolic process | | | | | |
| (GO:0019220) | | | | | |
| protein | 0.589305 | 0.545963 | 0.482436 | 0.609629 | 0.691022 |
| modification by | | | | | |
| small protein | | | | | |
| conjugation or | | | | | |
| removal | | | | | |
| (GO:0070647) | | | | | |
| regulation of TOR | 0.61946 | 0.613018 | 0.642765 | 0.615441 | 0.64437 |
| signaling | | | | | |
| (GO:0032006) | | | | | |
| positive regulation | 0.661209 | 0.666797 | 0.671361 | 0.664135 | 0.680203 |
| of protein | | | | | |
| modification | | | | | |
| process | | | | | |
| (GO:0031401) | | | | | |
| cellular component | 0.587513 | 0.582045 | 0.583439 | 0.66554 | 0.739466 |
| organization or | | | | | |
| biogenesis | | | | | |
| (GO:0071840) | | | | | |
| positive regulation | 0.655383 | 0.68571 | 0.687107 | 0.717606 | 0.683492 |
| of cell | | | | | |
| communication | | | | | |
| (GO:0010647) | | | | | |
| protein lipidation | 0.708031 | 0.658792 | 0.613979 | 0.65792 | 0.658983 |
| (GO:0006497) | | | | | |
| cytokinetic process | 0.626688 | 0.630035 | 0.631038 | 0.601487 | 0.619564 |
| (GO:0032506) | | | | | |
| protein localization | 0.608564 | 0.606044 | 0.602636 | 0.626746 | 0.66459 |
| to phagophore | | | | | |
| assembly site | | | | | |
| (GO:0034497) | | | | | |
| small GTPase- | 0.572287 | 0.564827 | 0.561933 | 0.703554 | 0.621623 |
| mediated signal | | | | | |
| transduction | | | | | |
| (GO:0007264) | | | | | |
| nephron | 0.614221 | 0.614202 | 0.615046 | 0.604841 | 0.629976 |
| development | | | | | |
| (GO:0072006) | | | | | |
| regulation of gene | 0.593977 | 0.58678 | 0.588214 | 0.651021 | 0.663482 |
| expression | | | | | |
| (GO:0010468) | | | | | |
| programned cell | 0.625884 | 0.627727 | 0.631568 | 0.657283 | 0.63708 |
| death | | | | | |
| (GO:0012501) | | | | | |
| regulation of | 0.598369 | 0.592817 | 0.594238 | 0.637278 | 0.650364 |
| cellular biosynthetic | | | | | |
| process | | | | | |
| (GO:0031326) | | | | | |
| regulation of | 0.62418 | 0.625493 | 0.624776 | 0.595913 | 0.655632 |
| macroautophagy | | | | | |
| (GO:0016241) | | | | | |
| positive regulation | 0.612653 | 0.612658 | 0.613498 | 0.848442 | 0.758076 |
| of cell cycle process | | | | | |
| (GO:0090068) | | | | | |
| endomembrane | 0.626868 | 0.630605 | 0.631551 | 0.613729 | 0.644929 |
| system organization | | | | | |
| (GO:0010256) | | | | | |
| regulation of | 0.604802 | 0.588378 | 0.60547 | 0.633653 | 0.681415 |
| mitotic | | | | | |
| metaphase/anaphase | | | | | |
| transition | | | | | |
| (GO:0030071) | | | | | |
| regulation of | 0.599183 | 0.594051 | 0.595354 | 0.558805 | 0.646571 |
| biosynthetic process | | | | | |
| (GO:0009889) | | | | | |
| regulation of | 0.61134 | 0.611341 | 0.612554 | 0.627998 | 0.663136 |
| synapse | | | | | |
| organization | | | | | |
| (GO:0050807) | | | | | |
| positive regulation | 0.642072 | 0.666729 | 0.653487 | 0.689983 | 0.655805 |
| of signal | | | | | |
| transduction | | | | | |
| (GO:0009967) | | | | | |
| skeletal system | 0.61525 | 0.615219 | 0.616064 | 0.599429 | 0.61067 |
| development | | | | | |
| (GO:0001501) | | | | | |
| positive regulation | 0.606567 | 0.602735 | 0.603946 | 0.648789 | 0.62327 |
| of chromosome | | | | | |
| segregation | | | | | |
| (GO:0051984) | | | | | |
| positive regulation | 0.612998 | 0.612997 | 0.613836 | 0.259606 | 0.451283 |
| of cell cycle | | | | | |
| (GO:0045787) | | | | | |
| regulation of | 0.6287 | 0.631315 | 0.629496 | 0.649865 | 0.640565 |
| metaphase plate | | | | | |
| congression | | | | | |
| (GO:0090235) | | | | | |
| intracellular protein | 0.656398 | 0.677038 | 0.672096 | 0.635754 | 0.646681 |
| transport | | | | | |
| (GO:0006886) | | | | | |
| regulation of DNA | 0.611283 | 0.611317 | 0.612104 | 0.728155 | 0.748019 |
| metabolic process | | | | | |
| (GO:0051052) | | | | | |
| autophagosome | 0.606192 | 0.604006 | 0.607021 | 0.61772 | 0.622637 |
| organization | | | | | |
| (GO:1905037) | | | | | |
| spindle organization | 0.612118 | 0.61213 | 0.612976 | 0.647289 | 0.678401 |
| (GO:0007051) | | | | | |
| DNA repair | 0.568483 | 0.566913 | 0.568694 | 0.675552 | 0.715385 |
| (GO:0006281) | | | | | |
| cell growth | 0.523757 | 0.522566 | 0.529669 | 0.677715 | 0.679817 |
| (GO:0016049) | | | | | |
| regulation of | 0.548175 | 0.539776 | 0.541678 | 0.585518 | 0.676498 |
| transferase activity | | | | | |
| (GO:0051338) | | | | | |
| animal organ | 0.530776 | 0.536135 | 0.514541 | 0.614758 | 0.626276 |
| development | | | | | |
| (GO:0048513) | | | | | |
| regulation of | 0.675766 | 0.666872 | 0.708155 | 0.757354 | 0.732807 |
| biological quality | | | | | |
| (GO:0065008) | | | | | |
| embryo | 0.554772 | 0.541362 | 0.544968 | 0.629302 | 0.651752 |
| development | | | | | |
| (GO:0009790) | | | | | |
| in utero embryonic | 0.614596 | 0.67672 | 0.761009 | 0.686936 | 0.607785 |
| development | | | | | |
| (GO:0001701) | | | | | |
| positive regulation | 0.576602 | 0.573107 | 0.576112 | 0.574173 | 0.59635 |
| of protein | | | | | |
| localization | | | | | |
| (GO:1903829) | | | | | |
| regulation of | 0.586617 | 0.582352 | 0.58352 | 0.629742 | 0.720114 |
| cytoskeleton | | | | | |
| organization | | | | | |
| (GO:0051493) | | | | | |
| chordate embryonic | 0.614009 | 0.614002 | 0.614843 | 0.620714 | 0.639331 |
| development | | | | | |
| (GO:0043009) | | | | | |
| supramolecular | 0.675862 | 0.704483 | 0.704094 | 0.708186 | 0.731703 |
| fiber organization | | | | | |
| (GO:0097435) | | | | | |
| regulation of cell | 0.592029 | 0.591992 | 0.595043 | 0.639668 | 0.651795 |
| growth | | | | | |
| (GO:0001558) | | | | | |
| microtubule | 0.599442 | 0.594559 | 0.594139 | 0.617584 | 0.617658 |
| cytoskeleton | | | | | |
| organization | | | | | |
| (GO:0000226) | | | | | |
| synaptic vesicle | 0.625564 | 0.661797 | 0.733273 | 0.620377 | 0.639 |
| endocytosis | | | | | |
| (GO:0048488) | | | | | |
| meiotic cell cycle | 0.613558 | 0.613549 | 0.614407 | 0.629735 | 0.64813 |
| process | | | | | |
| (GO:1903046) | | | | | |
| cell development | 0.590746 | 0.597444 | 0.583673 | 0.618954 | 0.640622 |
| (GO:0048468) | | | | | |
| negative regulation | 0.573151 | 0.559122 | 0.553916 | 0.681939 | 0.719408 |
| of cell | | | | | |
| communication | | | | | |
| (GO:0010648) | | | | | |
| anatomical structure | 0.571267 | 0.572541 | 0.573135 | 0.595891 | 0.62866 |
| morphogenesis | | | | | |
| (GO:0009653) | | | | | |
| mitotic | 0.584816 | 0.603769 | 0.59476 | 0.794624 | 0.763346 |
| chromosome | | | | | |
| condensation | | | | | |
| (GO:0007076) | | | | | |
| positive regulation | 0.618095 | 0.618118 | 0.61978 | 0.586184 | 0.641502 |
| of catalytic activity | | | | | |
| (GO:0043085) | | | | | |
| protein localization | 0.613059 | 0.613046 | 0.623998 | 0.566712 | 0.563827 |
| to cell periphery | | | | | |
| (GO:1990778) | | | | | |
| cell surface receptor | 0.60708 | 0.60291 | 0.603599 | 0.76566 | 0.810652 |
| signaling pathway | | | | | |
| (GO:0007166) | | | | | |
| negative regulation | 0.613027 | 0.620325 | 0.675719 | 0.604183 | 0.66726 |
| of organelle | | | | | |
| organization | | | | | |
| (GO:0010639) | | | | | |
| mitotic cytokinesis | 0.613256 | 0.613253 | 0.6141 | 0.646639 | 0.622826 |
| (GO:0000281) | | | | | |
| regulation of | 0.597972 | 0.592952 | 0.594562 | 0.587058 | 0.604939 |
| developmental | | | | | |
| process | | | | | |
| (GO:0050793) | | | | | |
| embryo | 0.614014 | 0.614007 | 0.614848 | 0.620712 | 0.639341 |
| development ending | | | | | |
| in birth or egg | | | | | |
| hatching | | | | | |
| (GO:0009792) | | | | | |
| osteoblast | 0.612707 | 0.612707 | 0.613597 | 0.593196 | 0.6368 |
| differentiation | | | | | |
| (GO:0001649) | | | | | |
| microtubule-based | 0.592251 | 0.583533 | 0.583676 | 0.629717 | 0.646778 |
| process | | | | | |
| (GO:0007017) | | | | | |
| negative regulation | 0.632367 | 0.632574 | 0.639035 | 0.694399 | 0.725448 |
| of signaling | | | | | |
| (GO:0023057) | | | | | |
| regulation of | 0.6215 | 0.614841 | 0.790132 | 0.744414 | 0.665286 |
| protein localization | | | | | |
| to plasma | | | | | |
| membrane | | | | | |
| (GO:1903076) | | | | | |
| negative regulation | 0.559079 | 0.557409 | 0.568297 | 0.674583 | 0.630462 |
| of TORC1 signaling | | | | | |
| (GO:1904262) | | | | | |
| cytoskeleton- | 0.549012 | 0.530844 | 0.534709 | 0.581538 | 0.626296 |
| dependent | | | | | |
| cytokinesis | | | | | |
| (GO:0061640) | | | | | |
| negative regulation | 0.573959 | 0.57154 | 0.573713 | 0.712346 | 0.73248 |
| of transcription by | | | | | |
| RNA polymerase II | | | | | |
| (GO:0000122) | | | | | |
| intracellular signal | 0.61992 | 0.620064 | 0.62533 | 0.652897 | 0.72328 |
| transduction | | | | | |
| (GO:0035556) | | | | | |
| heart development | 0.614227 | 0.614213 | 0.615059 | 0.595405 | 0.632136 |
| (GO:0007507) | | | | | |
| positive regulation | 0.602465 | 0.605273 | 0.600015 | 0.617746 | 0.669699 |
| of cell | | | | | |
| differentiation | | | | | |
| (GO:0045597) | | | | | |
| ameboidal-type cell | 0.614541 | 0.614518 | 0.615339 | 0.639056 | 0.637112 |
| migration | | | | | |
| (GO:0001667) | | | | | |
| nervous system | 0.613203 | 0.613201 | 0.614041 | 0.601423 | 0.622367 |
| development | | | | | |
| (GO:0007399) | | | | | |
| regulation of cell | 0.613728 | 0.613721 | 0.614562 | 0.592989 | 0.636376 |
| adhesion | | | | | |
| (GO:0030155) | | | | | |
| cartilage | 0.614982 | 0.614959 | 0.615791 | 0.59745 | 0.611817 |
| development | | | | | |
| (GO:0051216) | | | | | |
| cell differentiation | 0.61476 | 0.614142 | 0.615306 | 0.620501 | 0.64511 |
| (GO:0030154) | | | | | |
| regulation of | 0.615218 | 0.615211 | 0.616041 | 0.55532 | 0.595321 |
| epithelial cell | | | | | |
| proliferation | | | | | |
| (GO:0050678) | | | | | |
| cell-cell signaling | 0.611707 | 0.61172 | 0.61257 | 0.70478 | 0.674387 |
| (GO:0007267) | | | | | |
| negative regulation | 0.612659 | 0.612661 | 0.613501 | 0.591653 | 0.620957 |
| of response to | | | | | |
| stimulus | | | | | |
| (GO:0048585) | | | | | |
| actin filament-based | 0.581995 | 0.575007 | 0.57232 | 0.636284 | 0.624932 |
| process | | | | | |
| (GO:0030029) | | | | | |
| regulation of | 0.613815 | 0.613807 | 0.614661 | 0.61401 | 0.643883 |
| vasculature | | | | | |
| development | | | | | |
| (GO:1901342) | | | | | |
| regulation of | 0.614699 | 0.614673 | 0.615511 | 0.594842 | 0.595357 |
| cellular response to | | | | | |
| growth factor | | | | | |
| stimulus | | | | | |
| (GO:0090287) | | | | | |
| nephron tubule | 0.613863 | 0.613852 | 0.614701 | 0.660778 | 0.653441 |
| development | | | | | |
| (GO:0072080) | | | | | |
| negative regulation | 0.545233 | 0.558233 | 0.502851 | 0.736107 | 0.555607 |
| of cellular response | | | | | |
| to growth factor | | | | | |
| stimulus | | | | | |
| (GO:0090288) | | | | | |
| negative regulation | 0.601305 | 0.599776 | 0.597625 | 0.645803 | 0.65151 |
| of signaling | | | | | |
| (GO:0023057) | | | | | |
| wound healing | 0.601439 | 0.598214 | 0.601118 | 0.634992 | 0.664846 |
| (GO:0042060) | | | | | |
| multicellular | 0.622124 | 0.622036 | 0.627494 | 0.645659 | 0.646471 |
| organismal process | | | | | |
| (GO:0032501) | | | | | |
| response to organic | 0.667303 | 0.638175 | 0.674024 | 0.698554 | 0.707427 |
| substance | | | | | |
| (GO:0010033) | | | | | |
| tissue | 0.576626 | 0.556593 | 0.560276 | 0.606878 | 0.555121 |
| morphogenesis | | | | | |
| (GO:0048729) | | | | | |
| cellular response to | 0.65431 | 0.633208 | 0.664236 | 0.687407 | 0.675967 |
| chemical stimulus | | | | | |
| (GO:0070887) | | | | | |
| regulation of | 0.612996 | 0.612996 | 0.613831 | 0.611688 | 0.607406 |
| intracellular signal | | | | | |
| transduction | | | | | |
| (GO:1902531) | | | | | |
| regulation of cell- | 0.601813 | 0.599386 | 0.601923 | 0.636764 | 0.681654 |
| substrate adhesion | | | | | |
| (GO:0010810) | | | | | |
| positive regulation | 0.612662 | 0.612663 | 0.61355 | 0.639514 | 0.672187 |
| of osteoblast | | | | | |
| differentiation | | | | | |
| (GO:0045669) | | | | | |
| intracellular | 0.634844 | 0.637078 | 0.647961 | 0.637086 | 0.639462 |
| signaling cassette | | | | | |
| (GO:0141124) | | | | | |
| cell death | 0.613834 | 0.61383 | 0.614667 | 0.64126 | 0.679238 |
| (GO:0008219) | | | | | |
| positive regulation | 0.643316 | 0.65814 | 0.691556 | 0.64174 | 0.736312 |
| of vasculature | | | | | |
| development | | | | | |
| (GO:1904018) | | | | | |
| cell-substrate | 0.614909 | 0.614889 | 0.615731 | 0.58348 | 0.60466 |
| adhesion | | | | | |
| (GO:0031589) | | | | | |

### REFERENCES

### Journal Articles & Conference Papers

[1] Alzubaidi, L., Fadhel, M. A., Al-Shamma, O., Zhang, J., & Duan, Y. (2023). Cervical cancer diagnosis using stacked ensemble model and optimized feature selection: An explainable artificial intelligence approach. Computers. 12 (10), 200. https://doi.org/10.3390/computers12100200
[2] Cilloniz, D., Messa, F., Rosso, V., et al. (2008). Increased sensitivity to chemotherapeutical agents and cytoplasmatic interaction between NPM leukemic mutant and NF-κB in AML carrying NPM1 mutations. Leukemia, 22, 1234-1240.
[3] Chang, T. P., Yu, S. L., Lin, S. Y., et al. (2010). Tumor suppressor HLJI binds and functionally alters nucleophosmin via activating enhancer binding protein 2α complex formation. Cancer Research, 70, 1656-1667.
[4] Chen, S., Zhou, Y., Frank, M., & Xia, R. (2018). fastp: An ultra-fast all-in-one FASTQ preprocessor. Bioinformatics, 34 (17), i884-1890. https://doi.org/10.1093/bioinformatics/bty560
[5] Davis, S., & Meltzer, P. (2007). GEOquery: A bridge between the Gene Expression Omnibus (GEO) and BioConductor. Bioinformatics, 23 (14), 1846-1847. https://doi.org/10.1093/bioinformatics/btm251
[6] Dobin. A., Davis, C. A., Schlesinger, F., & Drenkow, J. (2013). STAR: Ultrafast universal RNA-seq aligner. Bioinformatics, 29 (1). 15-21. https://doi.org/10.1093/bioinformatics/bts635
[7] Flynt, A. S., & Lai, E. C. (2008). Biological principles of microRNA-mediated regulation: Shared themes amid diversity. Nature Reviews Genetics, 9, 831-842.
[8] Guidotti, R., Monreale, A., Ruggieri, S., Pedreschi, D., Turini, F., & Giannotti, F. (2018). Local rule-based explanations of black box decision systems. https://doi.org/10.48550/arXiv.1805.10820
[9] Kostka, D., & Spang, R. (2004). Finding disease-specific alterations in the coexpression of genes. Bioinformatics, 20 (Suppl 1), 1194-1199.
[10] Lau, F. S. (2021)-“Co-expression analysis reveals ERBB2-and NPM1 associated gene network in relapse HER2-positive breast cancer.”
[11] Lee, T. I., & Young, R. A. (2013). Transcriptional regulation and its misregulation in disease. Cell, 152, 1237-1251.
[12] Li. B., & Dewey, C. N. (2011). RSEM: Accurate transcript quantification from RNA-Seq data with or without a reference genome. BMC Bioinformatics, 12, 323. https://doi.org/10.1186/1471-2105-12-323
[13] Li, Z., Boone, D., & Hann, S. R. (2008). Nucleophosmin interacts directly with c-Myc and controls c-Mye-induced hyperproliferation and transformation. PNAS, 105. 18794-18799.
[14] Lin, S., & Gregory, R. I. (2015). MicroRNA biogenesis pathways in cancer. Nature Reviews Cancer, 15, 321-333.
[15] Liu, Y., Zhang, F., Zhang, X. F., et al. (2012). Expression of nucleophosmin/NPM1 correlates with migration and invasiveness of colon cancer cells. Journal of Biomedical Science, 19, 53.
[16] Liu, Y., Zhang, Y., Wang, Y., Wang, Y., & Li. X. (2022). Application of machine learning in the prediction of deficient mismatch repair in colorectal cancer. Frontiers in Oncology, 12, 1049305. https://doi.org/10.3389/fonc.2022.1049305
[17] Love, M. I., Huber, W., & Anders, S. (2014). Moderated estimation of fold change and dispersion for RNA-seq data with DESeq2. Genome Biology, 15 (12), 550. https://doi.org/10.1186/s13059-014-0550-8
[18] Miller, D. M., Thomas, S. D., Islam, A., Muench, D., & Sedoris, K. (2012). c-Myc and cancer metabolism. Clinical Cancer Research, 18, 5546-5553.
[19] Mothilal, R. K., Sharma, A., & Tan, C. (2019). Explaining machine learning classifiers through diverse counterfactual explanations. https://doi.org/10.48550/arXiv.1905.07697
[20] Pianta, A., Puppin, C., Passon, N., et al. (2011). Nucleophosmin delocalization in thyroid tumour cells. Endocrine Pathology, 22, 18-23.
[21] Ribeiro, M. T., Singh, S., & Guestrin, C. (2018). Anchors: High-precision model-agnostic explanations. Proceedings of the AAAI Conference on Artificial Intelligence, 32(1). https://doi.org/10.1609/aaai.v32il.11491
[22] Ribeiro, M. T., Singh, S., & Guestrin, C. (2016). Why should I trust you?: Explaining the predictions of any classifier. In Proceedings of the 22nd ACM SIGKDD International Conference on Knowledge Discovery and Data Mining (pp. 1135-1144). https://doi.org/10.1145/2939672.2939778Riquelme Medina, I., & Lubovac-Pilav, Z. (2016). Gene co-expression network analysis in type 1 diabetes. PLOS ONE, 11, e0156006.
[23] Ritchie, M. E., Diyagama, D., Huang, Y. F., et al. (2015). limma: Linear models for microarray data. In Bioinformatics and Computational Biology Solutions Using R and Bioconductor (pp. 397-420). Springer. https://doi.org/10.1007/978-1-4939-2802-4_14
[24] Siwo, G. H., Tan, A., Button-Simons, K. A., et al. (2015). Predicting functional divergence of a drug resistance gene. [Journal Name], [Volume], [Pages].
[25] Tang, R., & Liu, H. (2019). Temporal networks of peripheral blood changes in Alzheimer's disease. Frontiers in Aging Neuroscience, 11, 83.
[26] Tsui, K. H., Juang, H. H., Lee, T. H., et al. (2008). Association of nucleophosmin/B23 with bladder cancer recurrence. Acta Pharmacologica Sinica, 29, 364-370.
[27] Wang, F., Chan, L. W., Cho, W. C., et al. (2014). Coexpression analysis of E2F1-3 and MYC target genes in CML. BioMed Research International, 2014, 439840.
[28] Wang, F., Wang, B., Long, J., Wang, F., & Wu, P. (2019). Identification of candidate target genes for endometrial cancer, such as ANOI, using weighted gene co-expression network analysis. Experimental and Therapeutic Medicine, 17, 298-306.
[29] Wang, G. X., Cho, K. W., Uhm, M., et al. (2014). Otopetrin 1 protects mice from obesity-associated metabolic dysfunction. Diabetes, 63, 1340-1352.
[30] Wang, Q., Ma, X., Chen, Y., et al. (2014). Identification of interferon-γ as a new target of liver X receptor. Biochemical Journal, 459, 345-354.
[31] Wang, W., Jiang, W., Hou, L., et al. (2017). Weighted gene co-expression network analysis of BMI. BMC Genomics, 18, 872.
[32] Wang, X., Michie, S. A., Xu, B., & Suzuki, Y. (2007). Importance of IFN-γ-mediated VCAM-1 expression. Journal of Interferon & Cytokine Research, 27, 329-338.
[33] Wang, Y., Murakami, Y., Yasui. T., et al. (2013). Significance of GPI-anchored protein enrichment in lipid rafts. Journal of Biological Chemistry, 288, 25490-25499.
[34] Wang, Z., Bao, W., Zou, X., et al. (2019). Co-expression analysis in contrast-induced acute kidney injury. PLOS ONE, 14, e0218574.
[35] Wu, Y., Liou, F., Luo, S., et al. (2019). Coexpression of key gene modules in breast cancer. Bioscience Reports, 39. [Article Number].
[36] Yang. L., & Shami, A. (2020). On hyperparameter optimization of machine learning algorithms. Neurocomputing, 415, 295-316. https://doi.org/10.1016/j.neucom.2020.07.061
[37] Zhang, H., Wang, Y., Li, X., & Chen, Y. (2024). An efficient interpretable stacking ensemble model for lung cancer prognosis. Computational Biology and Chemistry, 108, 108248. https://doi.org/10.1016/j.compbiolchem.2024.108248

### Software & Online Resources

[38] BorgwardtLab. (2022). reComBat [Computer software]. GitHub. https://github.com/BorgwardtLab/reComBat
[39] Carlson, M. (2019). org.Hs.eg.db [R package]. Bioconductor. https://bioconductor.org/packages/release/data/annotation/html/org.Hs.eg.db.html
[40] CatBoost team. (2024). CatBoost [Software]. https://catboost.ai/
[41] GeeksforGeeks. (2023 Oct. 16). Sklearn model hyper-parameters tuning. https://www.geeksforgeeks.org/skleam-model-hyper-parameters-tuning/
[42] Google Developers. (n.d.). Machine learning education. Retrieved Jun. 10, 2024, from https://developers.google.com/machine-learning
[43] Microsoft. (2024). LightGBM [Software]. https://lightgbm.readthedocs.io/en/latest/Parameters-Tuning.html
[44] Morgan, M., & McCallum, K. (2020). AnnotationDbi [R package]. Bioconductor. https://bioconductor.org/packages/release/bioc/html/AnnotationDbi.html
[45] Pandian, S. (2022 February). Hyperparameter tuning guide. Analytics Vidhya. https://www.analyticsvidhya.com/blog/2022/02/a-comprehensive-guide-on-hyperparameter-tuning-and-its-techniques/
[46] scikit-learn developers. (2024). Cross-validation [Documentation]. https://scikit-learn.org/stable/modules/cross_validation.html
[47] scikit-learn developers. (2024). Grid search [Documentation]. https://scikit-learn.org/stable/modules/grid_search.html
[48] scikit-learn developers. (2024). PCA [Documentation]. https://scikit-learn.org/stable/modules/generated/sklearn.decomposition.PCA.html
[49] scikit-learn developers. (2024). StandardScaler [Documentation]. https://scikit-learn.org/stable/modules/generated/sklearn.preprocessing.StandardScaler.html
[50] scikit-leam developers. (2024). t-SNE [Documentation]. https://scikit-learn.org/stable/modules/generated/sklearn.manifold.TSNE.html
[51] Singh, T. (2025 Mar. 11). Hyperparameter tuning. GeeksforGeeks. https://www.geeksforgeeks.org/hyperparameter-tuning/
[52] theislab. (2024). KBET [Computer software]. GitHub. https://github.com/theislab/kBET
[53] Wang, Y., & Irizarry, R. A. (2011). oligo [R package]. Bioconductor. https://bioconductor.org/packages/release/bioc/html/oligo.html
[54] Wikipedia. (2025 Mar. 4). Hyperparameter optimization. https://en.wikipedia.org/wiki/Hyperparameter_optimization
[55] Wikipedia. (a.d.). Square root. Retrieved Jun. 10, 2024, from https://en.wikipedia.org/wiki/Square_root
[56] XGBoost developers. (2024). XGBoost [Software]. https://xgboost.readthedocs.io/en/latest/python/sklearn_estimator.html

What is claimed is:

1. A method for analyzing biological pathways associated with a state of interest, the method comprising:
a. receiving a first gene expression dataset from cells in a state of interest;
b. receiving a second gene expression dataset from cells in a reference state;
c. detecting dysregulated gene sets related to the state of interest using whole-genome co-expression network analysis and differential gene expression analysis;
d. generating state-specific pathways using functional enrichment analysis on said dysregulated gene sets;
e. generating a dysregulated pathway score for each state-specific pathway using a machine learning model comprising a two-layer ensemble approach, wherein:
i. a first layer predicts states of interest based on the state-specific pathways using classifiers selected based on optimal performance metrics:
ii. each state-specific pathway is associated with a state of interest severity probability in the first layer;
iii. a second layer integrates probabilities from the first layer and computes a final state of interest classification using a stacking classifier;
iv. the severity probability of each state-specific pathway is used to assign a weight to that state-specific pathway in the final classification; and
v. the weight of each state-specific pathway is multiplied by that state-specific pathway's probability, generating a dysregulated pathway score for each state-specific pathway:
f. scaling and normalizing said dysregulated pathway scores, wherein higher scores indicate a greater likelihood of contribution to the state of interest; and
g. generating values indicating the impact of each gene on the state-specific pathway's contribution to the final state of interest classification at the model-wide and sample-specific levels.

2. The method of claim 1, wherein the state of interest is selected from a group of diseases, the group comprising:
a. cancers;
b. neurodegenerative diseases;
c. autoimmune diseases;
d. cardiovascular diseases;
e. infectious diseases;
f. aging-related diseases;
g. hematological diseases; and
h metabolic disorders.

3. The method of claim 1, wherein the functional enrichment analysis is performed using publicly available online platforms to identify biological processes associated with the state of interest.

4. The method of claim 1, wherein the gene expression data is obtained through RNA sequencing, microarrays, or retrieved from publicly available data repositories.

5. The method of claim 1 further comprising preprocessing steps selected from the group comprising:
a. quality control, transcript alignment;
b. gene count quantification, normalization; and
c. gene annotation prior to functional enrichment analysis.

6. The method of claim 1, wherein the machine learning model is:
a. trained using a training dataset of gene expression data and known disease states; and
b. validated using performance metrics comprising cross-validation.

7. The method of claim 1 further comprising generating a recommendation for therapeutic intervention based on dysregulated pathway scores and the predicted efficacy of available drugs or treatments for the pathway, wherein said therapeutic intervention is selected from the group comprising:
a. small molecule drugs;
b. biologics;
c. gene therapies;
d. cell-based therapies;
e. immunotherapies:
f. combination therapies;
g. targeted radiotherapies;
h. dietary or lifestyle interventions; and
i. alternative therapeutic options.

8. The method of claim 1 further comprising:
a. validating treatment efficacy by comparing pre-treatment and post-treatment dysregulated pathway scores; and
b. generating an adjusted treatment recommendation if a subject's dysregulated pathway score changes.

9. The method of claim 1 further comprising deriving a state of interest severity score from the dysregulated pathway score.

10. The method of claim 9 further comprising:
a. generating a personalized treatment recommendation based on state of interest severity score;
b. generating a recommendation for the administration of the personalized treatment based on state of interest severity score; and
c. ranking patients for prioritized personalized treatment based on state of interest severity score.

11. The method of claim 9 further comprising:
a. monitoring longitudinal changes in a subject's state of interest severity scores; and
b. generating an adjusted treatment recommendation if the subject's state of interest severity scores changes.

12. The method of claim 1 further comprising detecting molecular targets for personalized treatment using the values indicating the impact of each gene on the state-specific pathway's contribution to the final state of interest classification.

13. The method of claim 1, wherein the values indicating the impact of each gene on the state-specific pathway's contribution to the final state of interest classification are Shapley Additive Explanations values.

14. The method of claim 13, wherein the Shapley Additive Explanations values provide global interpretability by identifying genes that influence state of interest classification across the entire dataset, and local interpretability by providing a detailed breakdown of gene-level contributions for each individual sample.

15. The method of claim 13, wherein the machine learning model and Shapley Additive Explanations generating steps are subject-independent, allowing for the generation of personalized treatment strategies for an individual subject based on gene expression data.

16. A personalized treatment method for a state of interest, the method comprising:
a. receiving a first gene expression dataset from cells in a state of interest;
b. receiving a second gene expression dataset from cells in a reference state;
c. detecting dysregulated gene sets related to the state of interest using whole-genome co-expression network analysis and differential gene expression analysis;
d. generating state-specific pathways using functional enrichment analysis on said dysregulated gene sets;
e. generating a dysregulated pathway score for each state-specific pathway using a machine learning model comprising a two-layer ensemble approach, wherein:
i. a first layer predicts states of interest based on the state-specific pathways using classifiers selected based on optimal performance metrics:
ii. each state-specific pathway is associated with a state of interest severity probability in the first layer;
iii. a second layer integrates probabilities from the first layer and computes a final state of interest classification using a stacking classifier;
iv. the severity probability of each state-specific pathway is used to assign a weight to that state-specific pathway in the final classification; and
v. the weight of each state-specific pathway is multiplied by that state-specific pathway's probability, generating a dysregulated pathway score for each state-specific pathway:
f. scaling and normalizing said dysregulated pathway scores, wherein higher scores indicate a greater likelihood of contribution to the state of interest;
g. generating values indicating the impact of each gene on the state-specific pathway's contribution to the final state of interest classification at the model-wide and sample-specific levels;
h. detecting molecular targets for personalized treatment using the values indicating the impact of each gene on the state-specific pathway's contribution to the final state of interest classification;
i. generating a recommended personalized treatment; and
j. administering the personalized treatment.

17. A system for analyzing biological pathways associated with a state of interest, the system comprising:
a. a processor;
b. memory; and
c. program instructions, stored in the memory, that upon execution by the processor cause the computing device to perform operations for analyzing biological pathways associated with a state of interest, said operations comprising the steps of:
i. receiving a first gene expression dataset from cells in a state of interest;
ii. receiving a second gene expression dataset from cells in a reference state;
iii. detecting dysregulated gene sets related to the state of interest using whole-genome co-expression network analysis and differential gene expression analysis;
iv. generating state-specific pathways using functional enrichment analysis on said dysregulated gene sets;
v. generating a dysregulated pathway score for each state-specific pathway using a machine learning model comprising a two-layer ensemble approach, wherein:
1. a first layer predicts states of interest based on the state-specific pathways using classifiers selected based on optimal performance metrics;
2. each state-specific pathway is associated with a state of interest severity probability in the first layer;
3. a second layer integrates probabilities from the first layer and computes a final state of interest classification using a stacking classifier;
4. the severity probability of each state-specific pathway is used to assign a weight to that state-specific pathway in the final classification; and
5. the weight of each state-specific pathway is multiplied by that state-specific pathway's probability, generating a dysregulated pathway score for each state-specific pathway;
vi. scaling and normalizing said dysregulated pathway scores, wherein higher scores indicate a greater likelihood of contribution to the state of interest; and
vii. generating values indicating the impact of each gene on the state-specific pathway's contribution to the final state of interest classification at the model-wide and sample-specific levels.

18. The system of claim 17 wherein said operations further comprise the step of detecting molecular targets for personalized treatment using the values indicating the impact of each gene on the state-specific pathway's contribution to the final state of interest classification.

19. The system of claim 17, wherein the values indicating the impact of each gene on the state-specific pathway's contribution to the final state of interest classification are Shapley Additive Explanations values.
